(19) United States
(12) Patent Application Publication
(10) Pub. No.: US 2024/0141441 A1
(43) Pub. Date: May 2, 2024

(54) PANEL AND METHODS FOR POLYNUCLEOTIDE DETECTION

(71) Applicant: BIOFIDELITY LTD, Cambridge (GB)

(72) Inventors: Magdalena STOLAREK-JANUSZKIEWICZ, Cambridge (GB); Ana Luisa Bras Dos Santos Ribeiro DA SILVA-WEATHERLEY, Cambridge (GB); Barnaby William BALMFORTH, Cambridge (GB)

(21) Appl. No.: 18/546,479

(22) PCT Filed: Feb. 16, 2022
(86) PCT No.: PCT/GB2022/050418

§ 371 Date: Aug. 15, 2023

(30) Foreign Application Priority Data
Feb. 16, 2021 (GB) 2102170.4

(51) Int. Cl. C12Q 1/6886; C12Q 1/6874

(52) U.S. Cl. CPC C12Q 1/6886; C12Q 1/6874; C12Q 1/682; C12Q 2600/154; C12Q 2600/156

(57) ABSTRACT

This invention relates to molecular systems for detecting a target polynucleotide sequence in a given nucleic acid analyte and methods of use thereof.

### FIELD OF THE INVENTION

This invention relates to molecular systems for detecting a target polynucleotide sequence in a given nucleic acid analyte and methods of use thereof.

The polymerase chain reaction (PCR) is a well-known and powerful technique for amplifying DNA or RNA present in laboratory and diagnostic samples to a point where they can be reliably detected and/or quantified. However, when applied for the purposes of investigating analyte samples containing low-levels of such molecules, it suffers from a number of limitations. First, whilst the technique can detect as little as a single target molecule, it is prone to generating false positive results due to unwanted amplification of other nucleic acid sequences present in the sample. This makes the choice of oligonucleotide primers used to initiate the reaction key; which in turn makes designing primers with the required level of specificity relatively complex. As a consequence, many PCR-based tests available on the market today have limited specificity.

A second drawback is that multiplexing of PCR-based methods is in practice limited to at most tens of target sequences (frequently no more than 10) with the avoidance of primer-primer interactions resulting in the need for relatively narrow operational windows.

Another issue is that, because the PCR reaction cycles in an exponential fashion, quantification of the target is difficult; small variations in the efficiency of the reaction having a huge impact on the amount of detectable material generated. Even with appropriate controls and calibrations in place, quantification is thus typically limited to an accuracy within a factor of around 3.

Finally, mutations in the region targeted for investigation by PCR amplification methods can have unwanted side effects. For example, there have been instances where FDA-approved tests have had to be withdrawn because the target organism underwent mutation in the genetic region targeted by the test primers resulting in large numbers of false negatives. Conversely, if a specific single nucleotide polymorphism (SNP) is targeted for amplification the PCR method will often give a false positive when the wild-type variant is present. Avoiding this requires very careful primer design and further limits the efficacy of multiplexing. This is particularly relevant when searching for panels of SNPs as is a common requirement in cancer testing/screening or companion diagnostics.

### SUMMARY

We have now developed an improved method which builds on our experience using the pyrophosphorolysis reaction employed in our earlier patents (WO20016590 A1, PCT/GB2020/053361, PCT/GB2020/053362, PCT/GB2020/053363, GB2020539.9 and GB2101176.2) overcome many of these limitations. In doing so, it harnesses the double-strand specificity of pyrophosphorolysis; a reaction which will not proceed efficiently with single-stranded oligonucleotide substrates or double-stranded substrates which include blocking groups or nucleotide mismatches. The new method allows for more confident calling of low concentrations of target sequences. Thus, according to the present invention, there is provided a molecular system for detecting a target polynucleotide sequence in a given nucleic acid analyte, the molecular system comprising a probe molecule (A₀) and a hybridised splint molecule (C), wherein:
a. A₀ has a 3′-end which is complementary to the target polynucleotide sequence, a loop region and a 5′-phosphate; and
b. C is hybridised to the 5′-end of A₀ and provides a single stranded 3′-overhang, wherein the single stranded 3′-overhang can hybridise to a region located 1 to 50 bases in the 5′ direction from the 3′-end of A₀.

There is further provided a method for detecting a target polynucleotide sequence in a given nucleic acid analyte, the method comprising taking a molecular system and:
i) introducing the molecular system to a sample;
ii) treating A₀ with an enzyme for pyrophosphorolysis thereby removing complementary nucleotides from the 3′-end of the A₀ that is fully hybridised to the target, to form shortened probe A₁;
iii) using C to displace the 3′-end of A₁ from the target;
iv) ligating the ends of the A₁ to form a circle using the pre-hybridised C, to form circular A₂; and
v) detecting the presence of A₂.

The analytes to which the method of the invention can be applied are those nucleic acids, such as naturally-occurring or synthetic DNA or RNA molecules, which include the target polynucleotide sequence(s) being sought. In one embodiment, the analyte will typically be present in an aqueous solution containing it and other biological material and in one embodiment the analyte will be present along with other background nucleic acid molecules which are not of interest for the purposes of the test. In some embodiments, the analyte will be present in low amounts relative to these other nucleic acid components. Preferably, for example where the analyte is derived from a biological specimen containing cellular material, prior to performing step (i) of the method some or all of these other nucleic acids and extraneous biological material will have been removed using sample-preparation techniques such as filtration, centrifuging, chromatography or electrophoresis. Suitably, the analyte is derived from a biological sample taken from a mammalian subject (especially a human patient) such as blood, plasma, sputum, urine, skin or a biopsy. In one embodiment, the biological sample will be subjected to lysis in order that the analyte is released by disrupting any cells present. In other embodiments, the analyte may already be present in free form within the sample itself; for example cell-free DNA circulating in blood or plasma.

### BRIEF DESCRIPTION OF DRAWINGS

FIG. 1: Results for Example 1, Effect of length of annealing region between probe and target on the optimum temperature for the pyrophosphorolysis reaction, showing the difference in Cq (dCq) values observed between wildtype and mutant-containing samples. The pyrophosphorolysis reaction was run between 30-50° C. The length of the annealing region is summarised in Table 1. The longer the length of the annealing region between the probe and the target molecule, the higher the optimum temperature. For a complementarity of 29 base pairs (SEQ ID 1/2) the optimum is between 46-50° C., for 22 base pairs 30-42° C. Too short of an annealing region can lead to a decrease in dCq (SEQ ID 11/12 and 13/14). With a 19 base pair length, it was not possible to detect a positive signal indicating the presence of the target molecule (SEQ ID 15/16).

FIG. 2: Results for Example 2, Effect of length of annealing region between probe, target and splint, showing the difference in Cq (dCq) values observed between wildtype and mutant-containing samples. Summary of the lengths tested can be found in Table 2. For probe SEQ ID 22 (FIG. 2A), the best performing splint sequence is SEQ ID 24. For probe SEQ ID 23 (FIG. 2B), the best performing splint sequence is SEQ ID 31. The closer the position of ligation point to the start of the probe sequence, the larger the value of dCq. There is a larger value of dCq when Probe SEQ ID 23 is used, compared with SEQ ID 22, as it has a longer region of complementarity with the target, 23 base pairs compared with 17 base pairs.

FIG. 3: Results for Example 3, showing the difference in Cq (dCq) values observed between wildtype and mutant-containing samples at two different mutant allele fractions (AF) for two different probe/splint combinations, SEQ ID NO 22/SEQ ID NO 24 and SEQ ID NO 23/SEQ ID NO 44. Probe SEQ ID 22 and 23 have 17 and 35 base length complementary regions with the target molecule, respectively. The higher the number of complementary bases/longer the length of the complementary region, the higher value of dCq seen for 0.1% and 0.5% AF concentrations of the target molecule.

FIG. 4a&b: Shows a cartoon of the method in action. The pre-hybridised probe sequences are introduced to the target. Where the end of A0 is fully complementary (FIG. 4a), the ends are shortened. The shortened ends of A1 can be displaced intramolecularly by the sequence C. C forms a splint across the ends of A1, allowing A1 to be made circular, forming circle products A2. Where the ends of A0 are not fully complementary to the target (FIG. 4b), the probes are only shortened as far as the mis-match. The end C is unable to displace the target from A1, and no circular products are formed.

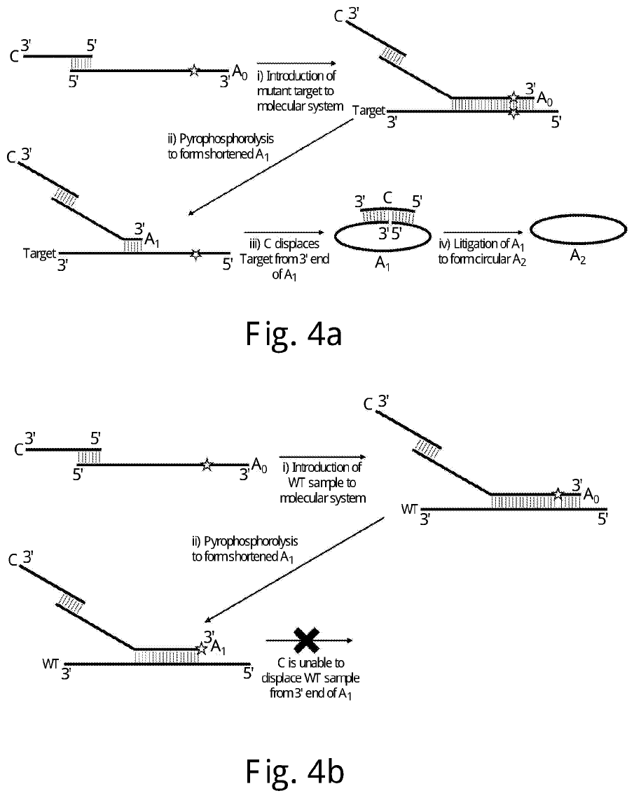

### DESCRIPTION OF EMBODIMENTS

In an aspect of the present invention, there is provided a molecular system for detecting a target polynucleotide sequence in a given nucleic acid analyte, the molecular system comprising a probe molecule (A₀) and a hybridised splint molecule (C), wherein:
a. A₀ has a 3′-end which is complementary to the target polynucleotide sequence, a loop region and a 5′-phosphate; and
b. C is hybridised to the 5′-end of A₀ and provides a single stranded 3′-overhang, wherein the single stranded 3′-overhang can hybridise to a region located 1 to 50 bases in the 5′ direction from the 3′-end of A₀.

In some embodiments, the 5′ end of A₀ is resistant to exonucleolysis.

In some embodiments, A₀ and C are hybridised at the 5′-end of A₀ across a region comprising a minimum of 5 complementary nucleotides.

In some embodiments, single stranded 3′-overhang of C is complementary to a region located 1-50 bases in the 5′ direction from the 3′-end of A₀ across a region comprising a minimum of 5 complementary nucleotides.

In some embodiments, the complementary regions are a minimum of 7 nucleotides in length.

In some embodiments, the 3′ end of A₀ is complementary to a region of a gene or chromosome within the DNA or RNA of a cancerous tumour cell.

In some embodiments, the 3′ end of A₀ is complementary to a region of a gene encoding a mutation found in non-small cell lung cancer (NSCLC).

In some embodiments, the 3′ end of A₀, or a portion of the 3′ end of A₀, is complementary to a gene as described subsequently.

In one embodiment, there is provided a panel comprising a plurality of molecular systems.

In one embodiment there is provided a panel comprising a plurality of molecular systems for detecting multiple target polynucleotide sequences in a given nucleic acid analyte, each of the plurality of molecular systems comprising a probe molecule (A₀) and a hybridised splint molecule (C), wherein:
a. each A₀ has a varying 3′-end which is complementary to one of the target polynucleotide sequences, a loop region and a 5′-phosphate; and
b. C is hybridised to the 5′-end of A₀ and provides a single stranded 3′-overhang having a varying sequence,

wherein the single stranded 3′-overhang can hybridise to a region located within the loop region 1 to 50 bases in the 5′ direction from the 3′-end of A0.

In one embodiment, there is provided a method for detecting a target polynucleotide sequence in a given nucleic acid analyte, the method comprising taking a molecular system as previously or subsequently described and:
i) introducing a sample to a reaction mixture comprising the molecular system;
ii) treating A₀ with an enzyme for pyrophosphorolysis thereby removing complementary nucleotides from the 3′-end of the A₀ that is fully hybridised to the target, to form shortened probe A₁;
iii) using C to displace the 3′-end of A₁ from the target;
iv) ligating the ends of the A₁ to form a circle using the pre-hybridised C, to form circular A₂; and
v) detecting the presence of A₂.

In one embodiment, there is provided a method for detecting target polynucleotide sequences in a given nucleic acid analyte, the method comprising taking a panel of molecular systems as described herein and:
i) introducing a sample to a reaction mixture comprising the panel of molecular systems;
ii) treating A₀ with an enzyme for pyrophosphorolysis thereby removing complementary nucleotides from the 3′-ends of A₀ that are fully hybridised to the targets, to form shortened probes A₁;
iii) using C to displace the 3′-ends of A₁ from the target;
iv) ligating the ends of A₁ to form circles using the pre-hybridised C, to form circular A₂; and
v) detecting the presence of A₂.

In some embodiments, the reaction mixture comprising the molecular system is referred to as a first reaction mixture.

In some embodiments, the detection of the presence of A₂ is achieved by the addition of the products of step (iv) to a second reaction mixture, said second reaction mixture comprising reagents allowing the detection of A₂.

In some embodiments, the reaction mixture comprising the molecular system further comprises the pyrophosphorolysis enzyme. In some embodiments, this reaction mixture further comprises a source of pyrophosphate ion.

In some embodiments, targeted regions of RNA present in the biological sample are reverse transcribed into DNA by a reverse transcriptase prior to introduction to the reaction mixture comprising the molecular system. In some embodiments, this is achieved via the use of a reverse transcriptase and appropriate nucleotides.

In some embodiments, targeted regions of RNA present in the sample is transcribed into DNA at the same time as a amplification via PCR of targeted nucleic acids present in the sample.

In some embodiments, the transcription of any targeted regions of RNA present in the sample into DNA occurs in a separate step to any amplification via PCR of target nucleic acids present in the sample.

In some embodiments of any of the previously, or subsequently, described methods, targeted regions of RNA present in the sample are not transcribed into DNA.

In such an embodiment, A₀ undergoes pyrophosphorolysis against an RNA sequence to form partially digested strand A₁ and the method then proceeds as previously, or subsequently, described.

In some embodiments, the target polynucleotide comprises a site of genetic mutation and this mutation is present at a low level in the sample compared to the wild-type sequences.

In some embodiments, A₂ is between 20 and 200 nucleotides in length.

In some embodiments, A₂ is between 40 and 100 nucleotides in length.

In some embodiments, after (iv) an exonuclease is used to digest any non-circularised nucleic acid material.

In some embodiments, A₀ has a 5′ end which is resistant to exonucleolysis and a 5′-3′ exonuclease is used to digest any nucleic acid molecules which are not rendered resistant to this exonucleolysis.

In some embodiments, the exonuclease used has activity at least partly dependent on the presence of a 5′ phosphate group and in that the digestion is carried out in the presence of a kinase and a phosphate donor.

In some embodiments, step (ii) is carried out in the presence of a phosphatase or diphosphohydrolase.

In some embodiments, the pyrophosphorolysis reaction is stopped after step (ii) through addition of a pyrophosphatase.

In some embodiments, following step (iv) the detection of A₂ is via nucleic acid amplification.

In some embodiments, step (v) comprises using one or more oligonucleotide binding dyes or molecular probes.

In some embodiments, multiple molecular systems are employed, each comprising A₀ selective for a different target sequence and each A₀ including an identification region.

In some embodiments, the identification regions(s) are characterised using molecular probes or through sequencing.

In some embodiments, the identification region(s) are used as priming sites for nucleic acid amplification, enabling the presence and identity of A₂ to be determined.

In some embodiments, (v) further comprises the steps of:
i. labelling the amplification products from A₂ using one or more oligonucleotide fluorescent binding dyes or molecular probes;
ii. measuring the fluorescent signal;
iii. exposing the amplification products from A₂ to a set of denaturing conditions; and
iv. identifying the polynucleotide target sequence in the analyte by monitoring changes in the fluorescent signal during exposure to the denaturing conditions.

In some embodiments, prior to step (ii) the analyte is split into multiple reaction volumes, each volume having a different molecular system comprising a different probe oligonucleotide A₀ introduced to detect a different target sequence, wherein the different probes A₀ comprise a common priming site for amplification, allowing a single or single set of amplification primers to be used for the detection of A₂.

In some embodiments, there is provided a kit comprising a molecular system and:
a ligase;
a pyrophosphorylising enzyme;
a source of ions suitable for driving the pyrophosphorolysis reaction; and
suitable buffers.

In some embodiments, the kit further comprises dNTPs and a polymerase.

In some embodiments, prior to (i) the sample is introduced to a reaction mixture which comprises one or more primers, deoxynucleotide triphosphates (dNTP) and an amplification enzyme and prior to step (i) the nucleic acid analytes present in a sample undergo amplification. In some embodiments the products of this amplification are subsequently treated with a proteinase prior to (i).

In some embodiments, the sample is treated with a proteinase prior to step (i). In some embodiments, the sample is treated with a proteinase during step (i). In some embodiments, the sample is treated with a proteinase after step (i).

In some embodiments the proteinase is heat inactivated prior to step (i).

In some embodiments, the second reaction mixture further comprises:
at least one single-stranded primer oligonucleotide, deoxynucleotide triphosphates (dNTPs) and an amplification enzyme; or
reagents suitable for the hybridisation chain reaction (HCR); or
reagents suitable for the ligation chain reaction (LCR);
wherein the pyrophosphorolysis enzyme is optionally the same enzyme which performs amplification.

In some embodiments, the deoxynucleotide triphosphates (dNTPs) are hot start dNTPs.

Hot start deoxynucleotide triphosphates (dNTPs) are dNTPs which are modified with a thermolabile protecting group at the 3′ terminus. The presence of this modification blocks DNA polymerase nucleotide incorporation until the nucleotide protecting group is removed using a heat activation step.

In an embodiment, the second reaction mixture, further comprises components for the hybridisation chain reaction (HCR).

In this embodiment, the second reaction mixture further comprises hairpin oligonucleotide 1 (HO1) and hairpin oligonucleotide 2 (HO2), each of which comprises a fluorophore and quencher such that when each oligonucleotide remains in a hairpin configuration the fluorophore and quencher are in contact with each other. HO1 is designed such that A₂ anneals to it, opening the ‘hairpin’ structure and separating the fluorophore from the quencher. The now ‘open’ HO1 is now able to anneal to HO2, opening the ‘hairpin’ structure and separating the fluorophore from the quencher.

In this embodiment, there are a plurality of hairpin oligonucleotides present such that the presence of one A₂ is able to cause a chain reaction of hairpin oligonucleotide opening resulting in the generation of a detectable fluorescent signal. This methodology is known in the literature as the Hybridisation Chain Reaction (HCR).

In some embodiments, the fluorophore of the fluorophore-quencher pair is selected from, but not limited to, dyes of the fluorescein family, the carboxyrhodamine family, the cyanine family, and the rhodamine family. Other families of dyes that can be used include, e.g., polyhalofluorescein-family dyes, hexachlorofluorescein-family dyes, coumarin-family dyes, oxazine-family dyes, thiazine-family dyes, squaraine-family dyes, chelated lanthanide-family dyes, the family of dyes available under the trade designation Alexa Fluor J, from Molecular Probes, the family of dyes available under the trade designation Atto from ATTO-TEC (Siegen, Germany) and the family of dyes available under the trade designation Bodipy J, from Invitrogen (Carlsbad, Calif.). Dyes of the fluorescein family include, e.g., 6-carboxyfluorescein (FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), 2′-chloro-7′-phenyl-1,4-dichloro-6-carboxyfluorescein (VIC), 6-carboxy-X-rhodamine (ROX), and 2′,4′,5′,7′-tetrachloro-5-carboxy-fluorescein (ZOE). Dyes of the carboxyrhodamine family include tetramethyl-6-carboxyrhodamine (TAMRA), tetrapropano-6-carboxyrhodamine (ROX), Texas Red, R110, and R6G. Dyes of the cyanine family include Cy2, Cy3, Cy3.5, Cy5, Cy5.5, and Cy7. Fluorophores are readily available commercially from, for instance, Perkin-Elmer (Foster City, Calif.), Molecular Probes, Inc. (Eugene, Oreg.), and Amersham GE Healthcare (Piscataway, N.J.).

In some embodiments, the quencher of the fluorophore-quencher pair may be a fluorescent quencher or a non-fluorescent quencher. Fluorescent quenchers include, but are not limited to, TAMRA, ROX, DABCYL, DABSYL, cyanine dyes including nitrothiazole blue (NTB), anthraquinone, malachite green, nitrothiazole, and nitroimidazole compounds. Exemplary non-fluorescent quenchers that dissipate energy absorbed from a fluorophore include those available under the trade designation Black Hole™ from Biosearch Technologies, Inc. (Novato, Calif.), those available under the trade designation Eclipse™. Dark, from Epoch Biosciences (Bothell, Wash.), those available under the trade designation Qx1J, from Anaspec, Inc. (San Jose, Calif.), those available under the trade designations ZEN and TAO from Integrated DNA Technologies (Coralville, Iowa) and those available under the trade designation Iowa Black™ from Integrated DNA Technologies (Coralville, Iowa).

In some embodiments, the fluorophore of the fluorophore-quencher pair may be fluorescein, Lucifer Yellow, B-phycoerythrin, 9-acridineisothiocyanate, Lucifer Yellow VS, 4-acetamido-4′-isothio-cyanatostilbene-2,2′-disulfonic acid, 7-diethylamino-3-(4′-isothiocyanatophenyl)-4-methylcoumarin, succinimdyl 1-pyrenebutyrate, and 4-acetamido-4′-isothiocyanatostilbene-2-,2′-disulfonic acid derivatives.

In some embodiments, the fluorophore of the fluorophore-quencher pair may be LC-Red 640, LC-Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate or other chelates of Lanthanide ions (e.g., Europium, or Terbium).

In some embodiments, the invention utilises fluorescently labelled oligonucleotides that are double quenched. The inclusion of a second, internal quencher shortens the distance between the dye and quencher and, in concert with the first quencher, provides greater overall dye quenching, lowering background and increasing signal detection. The second and first quenchers may be any of the quenchers previously described.

In an embodiment, the second reaction mixture, further comprises:
an oligonucleotide A comprising a substrate arm, a partial catalytic core and a sensor arm;
an oligonucleotide B comprising a substrate arm, a partial catalytic core and a sensor arm; and
a substrate comprising a fluorophore-quencher pair;
wherein the sensor arms of oligonucleotides A and B are complementary to flanking regions of A₂ such that in the presence of A₂ oligonucleotides A and B are combined to form a catalytically, multicomponent nucleic acid enzyme (MNAzyme). In this embodiment, the MNAzyme is formed only in the presence of A₂ and cleaves the substrate comprising a fluorophore-quencher pair such that a detectable fluorescent signal is generated.

In some embodiments, the fluorophore-quencher pair may be as described previously.

In some embodiments, the reaction mixtures of the invention are combined such that pyrophosphorolysis, ligation and the generation of a detectable fluorescent signal occurs without the addition of further reagents.

In an alternative embodiment, the second reaction mixture, further comprises a partially double-stranded nucleic acid construct wherein:
one strand comprises at least one RNA base, at least one fluorophore and wherein a region of this strand is complementary to a region of A₂ and wherein this strand may be referred to as the ‘substrate’ strand;
the other stand comprises at least one quencher and wherein a region of this strand is complementary to a region of A₂ adjacent to that which the substrate strand is complementary to, such that in the presence of A₂ the partially double stranded nucleic acid construct becomes substantially more double-stranded;

wherein in the process of becoming substantially more double-stranded the substrate strand of the double-stranded nucleic acid construct is cut at the RNA base, resulting in fluorescence due to the at least one quencher of the ‘other’ strand no longer being in close enough proximity to that of the at least one fluorophore of the substrate strand.

In other words, the partially double stranded nucleic acid construct, in the presence of A₂, has a double-stranded portion which is greater in size.

In some embodiments, the fluorophore-quencher pair may be as described previously.

In some embodiments, further reagents such as suitable buffers and/or ions are present in the second reaction mixture.

In some embodiments, the reaction mixture further comprises Mg²⁺ ions.

In some embodiments, the reaction mixture further comprises Zn²⁺ ions.

In some embodiments, the reaction mixture further comprises X²⁺ ions, wherein X is a metal.

In some embodiments, the reaction mixture further comprises one or more X²⁺ ions, wherein X is a metal.

In an alternative embodiment, the second reaction mixture, further comprises reagents for the ligase chain reaction (LCR).

In some embodiments, the second reaction mixture, comprises
a. one or more ligases; and
b. two or more LCR probe oligonucleotides that are complementary to adjacent sequences on A₂, wherein when the probes are successfully annealed to A₂ the 5′ phosphate of one LCR probe is directly adjacent to the 3′OH of the other LCR probe;

In some embodiments, in the presence of A₂ the two LCR probes will successfully anneal to A₂ and be ligated together to form one oligonucleotide molecule which subsequently acts as a new target for second-round covalent ligation, leading to geometric amplification of the target of interest, in this case A₂. The ligated products, or amplicons, are complementary to A₂ and function as targets in the next cycle of amplification. Thus, exponential amplification of the specific target DNA sequences is achieved through repeated cycles of denaturation, hybridization, and ligation in the presence of excess LCR probes. From this, the presence of A₂ and hence the target polynucleotide sequence is inferred.

In some embodiments, in the presence of A₂ the two PCR probes will successfully anneal to A₂ and be ligated together to form one oligonucleotide molecule which then acts as a new target for second-round covalent ligation, leading to geometric amplification of the target of interest, in this case A₂, which is then detected.

In some embodiments, the ligated oligonucleotide molecule is detected in real time using an intercalating dye.

In some embodiments, the ligated oligonucleotide molecule is detected using gel electrophoresis.

The person skilled in the art will appreciate there are numerous techniques which would allow the detection of the ligated oligonucleotide molecule.

In some embodiments, the deoxynucleotide triphosphates (dNTPs) are hot start dNTPs.

In some embodiments, the one or more ligases are thermostable.

In some embodiments, the one or more ligases are naturally occurring.

In another embodiment, the one or more ligases are engineered.

In some embodiments, the one or more ligases are selected from any ligase disclosed previously or subsequently.

In some embodiments, the one or more polymerases are thermostable.

In some embodiments, the one or more polymerases are selected from any polymerase disclosed previously or subsequently.

In some embodiments, the one or more polymerases are naturally occurring.

In another embodiment, the one or more polymerases are engineered.

In some embodiments, the one or more polymerases are the same as that used for the pyrophosphorolysis.

In some embodiments, one or more enzymes of the current invention are hot start enzymes.

In some embodiments, one or more enzymes of the current invention are thermostable.

In an alternate embodiment, the second reaction mixture, comprises:
a. a splint oligonucleotide, comprising a fluorophore-quencher pair, which is complementary to A₂;
b. a double strand specific DNA digestion enzyme;
wherein, in the presence of A₂, the splint oligonucleotide is digested such that the fluorophore-quencher pair are separated and a fluorescent signal, and hence the presence of A₂, is detectable.

In some embodiments, the fluorophore-quencher pair may be as described previously. In some embodiments, the double strand specific DNA digestion enzyme is an exonuclease. In another embodiment, it is a polymerase with proofreading activity. In another embodiment, the reaction mixture comprises a mixture of one or more of: an exonuclease or a polymerase with proofreading activity.

In some embodiments, the double strand specific DNA digestion enzyme is a hot start enzyme.

In some embodiments, the double strand specific DNA digestion enzyme has reduced activity at the temperature at which the pyrophosphorolysis reaction of the method takes place.

In some embodiments, the double strand specific DNA digestion enzyme has no activity at the temperature at which the pyrophosphorolysis reaction of the method takes place.

In some embodiments, the 3′ end of A₀ is perfectly complementary to the target polynucleotide sequence.

In some embodiments, the ligase is substantially lacking in single-strand ligation activity.

In some embodiments, the reaction mixture comprising partially digested strand A₁ is introduced to an inorganic pyrophosphatase prior to or during the detection step.

In the chemical sciences, methylation denotes the addition of a methyl group to a substrate or the substitution of an atom or group by a methyl group. Methylation is a form of alkylation with specifically a methyl group, rather than a larger carbon chain, replacing a hydrogen atom. These terms are commonly used in chemistry, biochemistry, soil science, and the biological sciences.

In biological systems, methylation is catalysed by enzymes: such methylation can be involved in modification of heavy metals, regulation of gene expression, regulation of protein function, and RNA metabolism. Methylation of heavy metals can also occur outside of biological systems. Chemical methylation of tissue samples is also one method for reducing certain histological staining artefacts.

Aberrant DNA methylation profiles have been associated many different complex disease states. In oncology, hypermethylation of tumour suppressor genes in serum DNA can be used as a diagnostic marker for small-cell lung cancer. In patients with immune disease, such as diabetes, rheumatoid arthritis (RA) and systemic lupus erythematosus (SLE), abnormal DNA methylation of cells of the immune system is found. Differential DNA methylation of peripheral blood leukocytes (PBL) in repetitive elements ALU, LINE-1 and Satellite 2 (measures for global DNA methylation), have been found to be associated with ischemic heart disease.

DNA methylation in vertebrates typically occurs at CpG sites (Cytosine-phosphate-guanine sites; that is, where a cytosine is directly followed by a guanine in the DNA sequence); this methylation results in the conversion of the cytosine to 5-methylcytosine. The formation of Me-CpG is catalysed by the enzyme DNA methyltransferase. The bulk of mammalian DNA has about 40% of CpG sites methylated but there are certain areas, known as CpG islands which are GC rich (made up of about 65% CG residues) where none are methylated. These are associated with the promoters of 56% of mammalian genes, including all ubiquitously expressed genes. 1-2% of the human genome is CpG clusters and there is an inverse relationship between CpG methylation and transcriptional activity.

DNA methylation involves the addition of a methyl group to the 5 position of cytosine pyrimidine ring or the 6 nitrogen of the adenine purine ring. This modification can be inherited through cell division. DNA methylation is typically removed during zygote formation and re-established through successive cell divisions during development. DNA methylation is a crucial part of normal organism development and cellular differentiation in higher organisms. DNA methylation stably alters the gene expression pattern in cells such that cells can “remember where they have been”; in other words, cells programmed to be pancreatic islets during embryonic development remain pancreatic islets throughout the life of the organisms without continuing signals telling them that they need to remain islets. In addition, DNA methylation suppresses the expression of viral genes and other deleterious elements which have been incorporated into the genome of the host over time. DNA methylation also forms the basis of chromatin structure, which enables cells to form the myriad characteristics necessary for multicellular life from a single immutable sequence of DNA. DNA methylation also plays a crucial role in the development of nearly all types of cancer.

Bisulfite sequencing is the use of bisulfite treatment of DNA to determine its pattern of methylation. DNA methylation was the first discovered epigenetic mark, and remains the most studied. It is also implicated in repression of transcriptional activity.

Among a number of mRNA modifications, N6-methyladenosine (m6A) modification is the most common type in eukaryotes and nuclear-replicating viruses. m6A has a significant role in numerous cancer types, including leukaemia, brain tumours, liver cancer, breast cancer and lung cancer.

Whilst 5-methylcytosine (5mC) is the most studied epigenetic modification, 5mC is oxidised to 5-hydroxymethylcytosine (5hmC) with the catalysis of TET (ten-eleven translocation) enzymes. Studies have shown that the distribution of 5hmC is tissue-specific, and there are differences in the distribution of 5hmC in different organs and tissues. The decreased expression of 5hmC in malignant tissue has been shown consistently in a wide range of different cancers, including melanoma. By evaluating a total of 15 pairs of normal and carcinoma samples in human breast tissue, the studies have shown that the levels of 5hmC were dramatically reduced in the cancer group compared with the healthy breast tissues.

Treatment of DNA with bisulfite converts cytosine residues to uracil, but leaves 5-methylcytosine residues unaffected. Thus, bisulfite treatment introduces specific changes in the DNA sequence that depend on the methylation status of individual cytosine residues, yielding single-nucleotide resolution information about the methylation status of a segment of DNA. Various analyses can be performed on the altered sequence to retrieve this information. The objective analysis is therefore reduced to differentiating between single nucleotide polymorphisms (cytosine's and thymines) resulting from bisulfite conversion. 5hmC converts to 5mC upon bisulfite treatment, which then reads as a C when sequenced, and thus cannot distinguish between 5hmC and 5mC. The output from bisulfite sequencing can no longer be defined as solely DNA methylation, as it is the composite of 5mC and 5hmC. The development of Tet-assisted oxidative bisulfite sequencing is now able to distinguish between the two modifications at single base resolution.

5hmC can be detected using TET-assisted bisulfite sequencing (TAB-seq). Fragmented DNA is enzymatically modified using sequential T4 Phage β-glucosyltransferase (T4-BGT) and then Ten-eleven translocation (TET) dioxygenase treatments before the addition of sodium bisulfite. T4-BGT glucosylates 5hmC to form beta-glucosyl-5-hydroxymethylcytosine (5ghmC) and TET is then used to oxidize 5mC to 5caC. Only 5ghmC is protected from subsequent deamination by sodium bisulfite and this enables 5hmC to be distinguished from 5mC by sequencing.

Oxidative bisulfite sequencing (oxBS) provides another method to distinguish between 5mC and 5hmC. The oxidation reagent potassium perruthenate converts 5hmC to 5-formylcytosine (5fC) and subsequent sodium bisulfite treatment deaminates 5fC to uracil. 5mC remains unchanged and can therefore be identified using this method.

APOBEC-coupled epigenetic sequencing (ACE-seq) excludes bisulfite conversion altogether and relies on enzymatic conversion to detect 5hmC. With this method, T4-BGT glucosylates 5hmC to 5ghmC and protects it from deamination by Apolipoprotein B mRNA editing enzyme subunit 3A (APOBEC3A). Cytosine and 5mC are deaminated by APOBEC3A and sequenced as thymine.

TET-assisted 5-methylcytosine sequencing (TAmC-seq) enriches for 5mC loci and utilizes two sequential enzymatic reactions followed by an affinity pull-down. Fragmented DNA is treated with T4-BGT which protects 5hmC by glucosylation. The enzyme mTET1 is then used to oxidize 5mC to 5hmC, and T4-BGT labels the newly formed 5hmC using a modified glucose moiety (6-N3-glucose). Click chemistry is used to introduce a biotin tag which enables enrichment of 5mC-containing DNA fragments for detection and genome wide profiling.

Methods of methylome analysis are broadly divided into 3 groups: restriction enzyme based, chromatin immunoprecipitation based (ChIP) or affinity based and bisulfite conversion (gene based). Restriction enzyme based methods are methylation-sensitive restriction enzymes for small/large scale DNA methylation analysis by combining the use of methylation-sensitive restriction enzymes experimental approaches (RLGS, DMH etc.) for global methylation analysis, applied to any genome without knowing the DNA sequence. However, large amounts of genomic DNA are required, making the method unsuitable for the analysis of samples when small amount of DNA is recovered. On the other hand, ChIP based methods are useful for the identification of differential methylated regions in tumours through the precipitation of a protein antigen out of a solution by using an antibody directed against the protein. These methods are protein based, applied extensively in cancer research.

Affinity enrichment is a technique that is often used to isolate methylated DNA from the rest of the DNA population. This is usually accomplished by antibody immunoprecipitation methods or with methyl-CpG binding domain (MBD) proteins.

Methylated DNA immunoprecipitation (MeDIP) is an antibody immunoprecipitation method that utilises a 5-methylcytidine antibody to specifically recognise methylated cytosines. The MeDIP kit requires the input DNA sample to be single-stranded in order for the 5-methylcytidine (5-mC) antibody to bind.

Another method for the enrichment of methylated DNA fragments uses recombinant methyl-binding protein MBD2b, or the MBD2b/MBD3L1 complex. One advantage of a methyl-CpG binding protein enrichment strategy is the input DNA sample does not need to be denatured; the protein can recognise methylated DNA in its native double-strand form. Another advantage is that the MBD protein binds only to DNA methylated in a CpG context to ensure the enrichment of methylated-CpG DNA, making this technique ideal for studying CpG islands.

In some embodiments, prior to, or during, step (i) of the methods of the invention, the one or more nucleic acid analytes are selectively modified.

In some embodiments, prior to, or during, step (i) the unmethylated cytosine bases in the one or more nucleic acid analytes are chemically or enzymatically converted.

In one embodiment, unmodified cytosine bases are converted to uracil by a methyltransferase enzyme.

In one embodiment, this enzyme is M.SssI.

In one embodiment, unmodified cytosine bases are converted to uracil by a deaminase enzyme.

An enzymatic methyl-seq workflow relies on the ability of APOBEC to deaminate cytosines to uracils. APOBEC also deaminates 5mC and 5hmC, making it impossible to differentiate between cytosine and its modified forms. In order to detect 5mC and 5hmC, this method also utilizes TET2 and an Oxidation Enhancer, which enzymatically modifies 5mC and 5hmC to forms that are not substrates for APOBEC. The TET2 enzyme converts 5mC to 5caC and the Oxidation Enhancer converts 5hmC to 5ghmC. Ultimately, cytosines are sequenced as thymines and 5mC and 5hmC are sequenced as cytosines, thereby protecting the integrity of the original 5mC and 5hmC sequence information.

In one embodiment, prior to, or during, step (i) the one or more nucleic acid analytes are introduced to an epigenetic modification-sensitive or epigenetic modification-dependent restriction endonuclease.

In one embodiment, the epigenetic modification-sensitive or epigenetic modification-dependent restriction endonuclease is McrBC.

In one embodiment, the epigenetic modification-sensitive or epigenetic modification-dependent restriction endonuclease is a member of the MspJI family.

In one embodiment, the endonuclease is AspBHI. In one embodiment, the endonuclease is FspEI.

In one embodiment, the endonuclease is LpnPI.

In one embodiment, the epigenetic modification-sensitive or epigenetic modification-dependent restriction endonuclease is a member of the PvuRts1I/AbaS family.

In one embodiment, the epigenetic modification-sensitive or epigenetic modification-dependent restriction endonuclease is a Type IIM endonuclease.

In one embodiment, the endonuclease is DpnI.

In one embodiment, the endonuclease is BisI.

In one embodiment, the epigenetic modification-sensitive or epigenetic modification-dependent restriction endonuclease is a Type IV endonuclease.

In one embodiment, the endonuclease is EcoKMcrBC.

In one embodiment, the endonuclease is SauUSI.

In one embodiment, the endonuclease is GmrSD.

In one embodiment, the epigenetic modification-sensitive or epigenetic modification-dependent restriction endonuclease is selected from the DpnII restriction endonuclease family.

In one embodiment, the endonuclease is DpnII.

In one embodiment, the endonuclease is DpnI.

In one embodiment, the epigenetic modification-sensitive or epigenetic modification-dependent restriction endonuclease is HpaI.

In one embodiment, the epigenetic modification-sensitive or epigenetic modification-dependent restriction endonuclease is HpaII.

In some embodiments, prior to, or during, step (i) the one or more nucleic acid analytes are introduced to a methylation-sensitive or methylation-dependent restriction endonuclease.

In some embodiments, prior to, or during, step (i) the one or more nucleic acid analytes are introduced to a methylation-sensitive or methylation-dependent restriction endonuclease followed by selective amplification of the target polynucleotide sequence containing the methylation status of interest through methylation-specific multiplex ligation-dependent probe amplification (MS-MLPA) of methylated DNA.

In some embodiments, prior to, or during, step (i) the population of methylated or unmethylated nucleic acid analytes is reduced.

In some embodiments, the reduction is carried out using methylated DNA immunoprecipitation (MeDIP).

In some embodiments, the reduction is carried out using methyl-binding proteins, such as MBD2b or the MBD2b/MBD3L1 complex.

It will be obvious to one skilled in the art that the present invention may be extended towards the detection of any epigenetic modification and is not limited to the detection of methylation status of target polynucleotide sequences. For example, the present invention could equally be adapted for the detection of other epigenetic modifications including hydroxymethylation—for example the hydroxylated form of 5mC (5-hmC). This recently appreciated form of epigenetic modification is an important epigenetic marker which influences gene expression and is distinct from CpG methylation. Other epigenetic modifications appear on RNA such as methyl adenosine and can be detected by methods of the invention.

In some embodiments the method according to invention is where the epigenetic modification is methylation. In further embodiments the epigenetic modification is methylation at CpG islands or by hydroxymethylation at CpG islands.

In some embodiments the epigenetic modification is methylation of adenine in either RNA or DNA.

In some embodiments, one or more oligonucleotides of the current invention are rendered resistant to pyrophosphorolysis and/or exonuclease digestion by the presence of one or more quenchers.

In some embodiments, after the addition of a suitable washing buffer, the resulting reaction mixture is mixed.

In some embodiments, the resulting reaction mixture is mixed by vortexing.

In some embodiments, the resulting reaction mixture is mixed by the movement of one or more magnetic beads present in the mixture.

In some embodiments, each wash step comprises the use of a washing buffer comprising one or more of: TrisHCL pH 7.5-8.0 5-20 mM, NaCL 0.4-2M, EDTA 0.1-1 mM and/or Tween20 0-0.1%.

In some embodiments of any of the previously, or subsequently, described methods, one or more reaction mixtures may be combined.

In some embodiments, the oligonucleotide C further comprises a 3′ or internal modification protecting it from 3′-5′ exonuclease digestion.

In some embodiments, the oligonucleotide C further comprises a 5′ modification protecting it from 5′-3′ exonuclease digestion.

In some embodiments, the oligonucleotide C further comprises a 3′ mismatch against A₀, or a 3′ modification, preventing the extension of oligonucleotide C.

In some embodiments, the method further comprises a two-step amplification performed between steps (iv) and (v). In some embodiments, the reaction volume is split into two or more separate volumes prior to the second amplification.

The person skilled in the art will understand there are a plethora of 3′ modifications which may be used to prevent extension.

In some embodiments, the second reaction mixture, further comprises a 5′-3′ exonuclease and the 5′ end of A₀ is rendered resistant to 5′-3′ exonuclease digestion.

In some embodiments, prior to or during the final step, the products of the previous step are treated with a pyrophosphatase.

In some embodiments, prior to or during the final step, the products of the previous step are treated with an exonuclease.

In some embodiments, detection is achieved using one or more oligonucleotide fluorescent binding dyes or molecular probes.

In some embodiments, an increase in signal over time resulting from the generation of amplicons of A₂ is used to infer the concentration of the target sequence in the analyte.

In some embodiments, multiple molecular systems comprising a probe molecule A₀ are employed, wherein each A₀ is selective for a different target sequence and includes an identification region, further characterised in that the amplicons of A₂ include the identification region and therefore the target sequences present in the analyte, are inferred through the detection of the identification region(s).

In some embodiments wherein multiple probes A₀ are employed, multiple blocking oligonucleotides are also employed.

In some embodiments, detection of the identification region(s) is carried out using molecular probes or through sequencing.

In some embodiments the identification region(s) are used as priming sites, enabling the detection and identification of A₂.

In some embodiments, the final step of the method further comprises the steps of:
v. labelling the products of the previous step using one or more oligonucleotide fluorescent binding dyes or molecular probes;
vi. measuring the fluorescent signal of the products;
vii. exposing the products to a set of denaturing conditions; and

identifying the polynucleotide target sequence in the analyte by monitoring changes in the fluorescent signal of the products during exposure to the denaturing conditions.

In some embodiments, the one or more nucleic acid analytes are split into multiple reaction volumes, each volume having a one or more molecular systems, introduced to detect different target sequences.

In some embodiments, the one or more nucleic acid analytes are split into multiple reaction volumes, each volume having one or more molecular systems.

In some embodiments, the different molecular systems comprising a probe molecule A₀ comprise common priming sites, allowing a single primer or single set of primers to be used for amplification of a region of A₂.

In an alternative embodiment, the second reaction mixture, further comprise one or more partially double stranded DNA constructs wherein each construct contains one or more fluorophores and one or more quenchers. In some embodiments, when the construct is partially double-stranded the one or more fluorophores and one or more quenchers are located in close enough proximity to each other such that sufficient quenching of the one or more fluorophores occurs.

In some embodiments, the construct is one strand of DNA with a self-complementary region that is looped back on itself.

In some embodiments, the construct comprises one primer of a primer pair.

In some embodiments, the second reaction mixture further comprises the other primer of a primer pair.

In some embodiments, a portion of the single stranded section of the construct hybridises to A₂ and is extended against it by a DNA polymerase. In some embodiments, the other primer of the primer pair then hybridises to the extended construct. This primer is then extended against the construct, displacing the self-complementary region. Thus, the one or more fluorophores and one or more dyes are separated sufficiently for a fluorescent signal to be detected, indicating the presence of A₂ in the reaction mixture.

In such an embodiment the construct may be known as a Sunrise Primer.

In some embodiments, the construct comprises two separate DNA strands.

In some embodiments, a portion of the single stranded section of the construct hybridises to A₂ and is extended against it by a DNA polymerase. In some embodiments, the other primer of the primer pair then hybridises to the extended construct. This primer is then extended against the construct, in the direction of the double stranded section, displacing the shorter of the DNA strands and thus the one or more fluorophores and one or more dyes are separated sufficiently for a fluorescent signal to be detected, indicating the presence of A₂ in the reaction mixture.

In such an embodiment the construct may be known as a Molecular Zipper.

The person skilled in the art will appreciate that for both the Sunrise Primer and Molecular Zipper it is possible for the one or more fluorophores and the one or more quencher pairs to be located at various positions within each respective construct. The key feature is that each pair is located in sufficient proximity to one another that in the absence of A₂, i.e. when no extension and strand displacement has occurred, no fluorescent signal is emitted.

In some embodiments according to any of the previously, or subsequently, described methods, RNA present in the sample is not transcribed to DNA. In such an embodiment, A₀ undergoes pyrophosphorolysis against an RNA sequence to form partially digested strand A₁ and the method then proceeds as previously, or subsequently, described.

In some embodiments of any of the previously, or subsequently, described methods, one or more reaction mixtures may be combined. According to the present invention, there are further provided methods of detecting a target polynucleotide sequence in a given nucleic acid analyte.

The analytes to which the various methods of the invention can be applied may be prepared from the biological sample mentioned above by a series of preliminary steps designed to amplify the analyte and separate it from the background genomic DNA which is typically present in significant excess.

In some embodiments, the target polynucleotide sequence in the analyte will be a gene or chromosomal region within the DNA or RNA of a cancerous tumour cell and will be characterised by the presence of one or more mutations; for example in the form of one or more single nucleotide polymorphisms (SNPs). Thus the invention will be useful in the monitoring of and/or treatment for disease recurrence. Patients who have been declared free of disease following treatment may be monitored over time to detect the recurrence of disease. This needs to be done non-invasively and requires sensitive detection of target sequences from blood samples. Similarly, for some cancers there are residual cancer cells that remain in a patient after treatment. Monitoring of the levels of these cells (or cell free DNA) present in the patient's blood, using the current invention, allows detection of recurrence of disease or failure of current therapy and the need to switch to an alternative.

In some embodiments, detection of the target polynucleotide sequence will allow repeated testing of patient samples during treatment of disease to allow early detection of developed resistance to therapy. For example, epidermal growth factor receptor (EGFR) inhibitors, such as gefitinib, erlotinib, are commonly used as first line treatments for non-small cell lung cancer (NSCLC). During treatment the tumour will often develop mutations in the EGFR gene (e.g T790M, C797S) which confer resistance to the treatment. Early detection of these mutations allows transfer of the patient onto alternative therapies.

In some embodiments, the target polynucleotide sequence in the analyte will be a gene or chromosomal region within the DNA or RNA of fetal origin and will be characterised by the presence of one or more mutations; for example in the form of one or more single nucleotide polymorphisms (SNPs). Thus, the invention may be used to detect mutations at very low allele fractions, at an earlier stage of pregnancy than other available testing techniques.

In another embodiment, the target polynucleotide sequence may be a gene or genomic region derived from an otherwise healthy individual but the genetic information obtained may assist in generating valuable companion diagnostic information allowing medical or therapeutic conclusions to be drawn across one or more defined groups within the human population.

In yet another embodiment, the target polynucleotide sequence may be characteristic of an infectious disease, or of resistance of an infectious disease to treatment with certain therapies; for example a polynucleotide sequence characteristic of a gene or chromosomal region of a bacterium or a virus, or a mutation therein conferring resistance to therapy.

In some embodiments, the target polynucleotide sequence may be characteristic of donor DNA. When a transplanted organ is rejected by the patient, the DNA from this organ is shed into the patient's bloodstream. Early detection of this DNA would allow early detection of rejection. This could be achieved using custom panels of donor-specific markers, or by using panels of variants known to be common in the population, some of which will be present in the donor and some in the recipient. Routine monitoring of organ recipients over time is thus enabled by the claimed method.

The success of organ transplantation can depend on the overall level of cumulative injury to the organ caused by several events in the donor. This includes age, lifestyle, ischemia/reperfusion injury (IRI) and immune response in the recipient. Research has shown that IRI causes epigenetic changes in the donor organ. The promoter region of the C3 gene becomes demethylated in the kidney, which is associated with chronic nephropathy post-transplantation. DNA methylation is a major contributor to a balanced immune response toward a graft as it regulates the function of cells of the immune system. Thus, detection of the methylation status of particular DNA sequences can allow identification of patients at risk for post-transplant complications.

In yet another embodiment, various versions of the method using different combinations of probes (see below) are employed in parallel so that the analyte can be simultaneously screened for multiple target sequences; for example sources of cancer, cancer indicators or multiple sources of infection. In this approach, the amplified products obtained by parallel application of the method are contacted with a detection panel comprised of one or more oligonucleotide binding dyes or sequence specific molecular probes such as a molecular beacon, hairpin probe or the like. Thus, in another aspect of the invention there is provided the use of at least one probe and optionally one ligation oligonucleotide in combination with one or more chemical and biological probes selective for the target polynucleotide sequences or with the use of sequencing to identify the amplified probe regions.

In some embodiments, the probe oligonucleotide A₀ of the molecular system comprises a priming region and a 3′ end which is complementary to the target polynucleotide sequence to be detected. By this means, a first intermediate product is created which is at least partially double-stranded. In some embodiments, this step is carried out in the presence of excess molecular system and in an aqueous medium containing the analyte and any other nucleic acid molecules.

During step (ii), the double-stranded region of the first intermediate product is pyrophosphorolysed in the 3′-5′ direction from the 3′ end of its A₀ strand. As a consequence, the A₀ strand is progressively digested to create a partially digested strand; hereinafter referred to as A₁. Where the probe oligonucleotide erroneously hybridises with a non-target sequence, the pyrophosphorolysis reaction will stop at any mismatches, preventing subsequent steps of the method from proceeding. In another embodiment, this digestion continues until A₁ lacks sufficient complementarity with the analyte or a target region therein to form a stable duplex. At this point, the various strands then separate by melting, thereby producing A₁. Under typical pyrophosphorolysis conditions, this separation occurs when there are between 6 and 20 complementary nucleotides between the analyte and A₀.

In another embodiment, the digestion continues until A₁ lacks sufficient complementarity with the analyte or target region therein for the pyrophosphorolyising enzyme to bind or for the pyrophosphorolyising reaction to continue. This typically occurs when there are between 6 and 20 complementary nucleotides remaining between the analyte and probe. In some embodiments, this occurs when there are between 6 and 40 complementary nucleotides remaining.

In an embodiment, the digestion continues until the length of complementarity between A₁ and the target is reduced to the point at which it is energetically favourable for oligo C to displace the analyte molecule from A₁. This typically occurs when the region of complementarity between A₁ and the analyte molecule is of similar or shorter length than the region of complementarity between oligo C and the 3′ end of A₁, but may also occur when the complementarity between A₁ and the analyte molecule is longer than this due to the favourability of intra-molecular hybridisation of the oligo C.

Suitably, pyrophosphorolysis is carried out in the reaction medium at a temperature in the range 20 to 90° C. in the presence of at least a polymerase exhibiting pyrophosphorolysis activity and a source of pyrophosphate ion. Further information about the pyrophosphorolysis reaction as applied to the digestion of polynucleotides can be found for example in J. Biol. Chem. 244 (1969) pp. 3019-3028 or our earlier patent application.

In some embodiments, the pyrophosphorolysis step is driven by the presence of a source of excess polypyrophosphate, suitable sources including those compounds containing 3 or more phosphorous atoms.

In some embodiments, the second reaction mixture comprises a source of excess polypyrophosphate.

In some embodiments, the pyrophosphorolysis step is driven by the presence of a source of excess modified pyrophosphate. Suitable modified pyrophosphates include those with other atoms or groups substituted in place of the bridging oxygen, or pyrophosphate (or polypyrophosphate) with substitutions or modifying groups on the other oxygens. The person skilled in the art will understand that there are many such examples of modified pyrophosphate which would be suitable for use in the current invention, a non-limiting selection of which are:

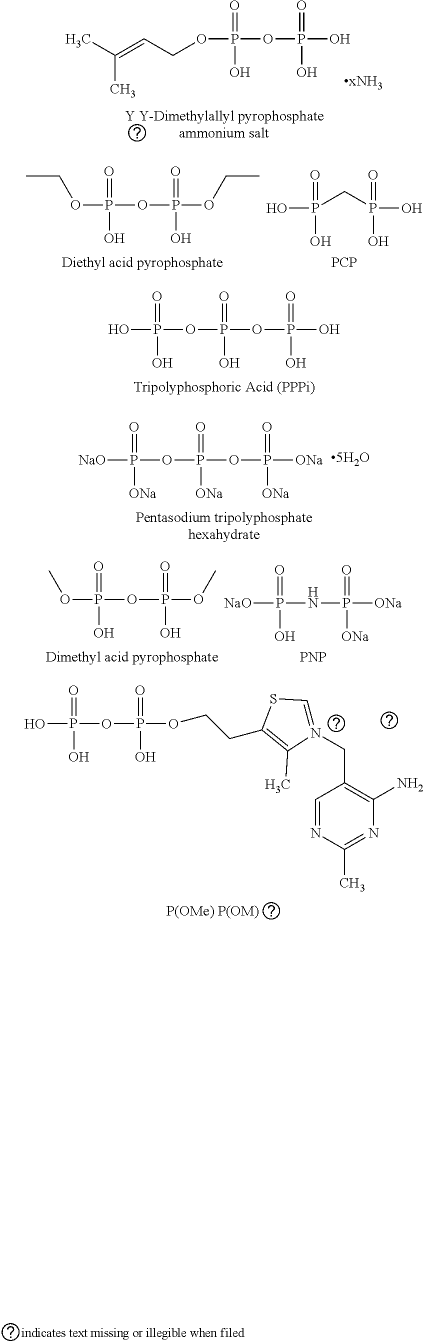

In some embodiments, the second reaction mixture comprises a source of excess modified polypyrophosphate.

In one preferred embodiment, the source of pyrophosphate ion is PNP, PCP or Tripolyphoshoric Acid (PPPi).

Further, but not limiting, examples of sources of pyrophosphate ion for use in the pyrophosphorolysis step (c) may be found in WO2014/165210 and WO00/49180.

In some embodiments, the source of excess modified pyrophosphate can be represented as Y—H wherein Y corresponds to the general formula (X—O)₂P(═B)—(Z—P(═B)(O—X))ₙ— wherein n is an integer from 1 to 4; each Z— is selected independently from —O—, —NH— or —CH₂—; each B is independently either O or S; the X groups are independently selected from —H, —Na, —K, alkyl, alkenyl, or a heterocyclic group with the proviso that when both Z and B correspond to —O— and when n is 1 at least one X group is not H.

In some embodiments, Y corresponds to the general formula (X—O)₂P(═B)—(Z—P(═B)(O—X))ₙ— wherein n is 1, 2, 3 or 4. In another embodiment, the Y group corresponds to the general formula (X—O)₂P(═O)—Z—P(═O)(O—H)— wherein one of the X groups is —H. In yet another preferred embodiment, Y corresponds to the general formula (X—O)₂P(═O)—Z—P(═O)(O—X)— wherein at least one of the X groups is selected from methyl, ethyl, allyl or dimethylallyl.

In an alternative embodiment, Y corresponds to either of the general formulae (H—O)₂P(═O)—Z—P(═O)(O—H)— wherein Z is either —NH— or —CH₂— or (X—O)₂P(═O)—Z—P(═O)(O—X)— wherein the X groups are all either —Na or —K and Z is either —NH— or —CH₂—.

In another embodiment, Y corresponds to the general formula (H—O)₂P(═B)—O—P(═B)(O—H)— wherein each B group is independently either O or S, with at least one being S.

Specific examples of preferred embodiments of Y include those of the formula (X1-O)(HO)P(═O)—Z—P(═O)(O—X2) wherein Z is O, NH or CH₂ and (a) X1 is γ,γ-dimethylallyl, and X2 is —H; or (b) X1 and X2 are both methyl; or (c) X1 and X2 are both ethyl; or (d) X1 is methyl and X2 is ethyl or vice versa.

In some embodiments, where molecular probes are to be used for detection, the probe oligonucleotide A₀ component of the molecular system is configured to include an oligonucleotide identification region on the 5′ side of the region complementary to the target sequence, and the molecular probes employed are designed to anneal to this identification region. In some embodiments, only the 3′ region of A₀ is able to anneal to the target; i.e. any other regions lack sufficient complementarity with the analyte for a stable duplex to exist at the temperature at which the pyrophosphorolysis step is carried out. Here and throughout, by the term ‘sufficient complementarity’ is meant that, to the extent that a given region has complementarity with a given region on the analyte, the region of complementarity is more than 10 nucleotides long.

In a further aspect of the methods of the invention there is provided alternate embodiments in which the phosphorolysis step of any previous embodiment is replaced with an exonuclease digestion step using a double-strand specific exonuclease. The person skilled in the art will understand that double-strand specific exonucleases include those that read in the 3′-5′ direction, such as ExoIII, and those that read in the 5′-3′ direction, such as Lambda Exo, amongst many others.

In some embodiments of the invention wherein the exonuclease digestion step utilises a double strand-specific 5′-3′ exonuclease, it is the 5′ end of A₀ that is complementary to the target analyte and the common priming sequence and blocking group are located on the 3′ side of the region complementary to the target. In a further embodiment, where molecular probes are to be used for detection the probe oligonucleotide A₀ component of the molecular system is configured to include an oligonucleotide identification region on the 3′ side of the region complementary to the target sequence, and the molecular probes employed are designed to anneal to this identification region.

In embodiments of the invention wherein the exonuclease digestion step utilises a double strand-specific 5′-3′ exonuclease, an exonuclease having 3′ to 5′ exonucleolytic activity can optionally be added to the second reaction mixture, for the purpose of digesting any other nucleic acid molecules present whilst leaving A₀ and any material comprising partially digested strand A₁ intact. Suitably, this resistance to exonucleolysis is achieved as described elsewhere in this application.

In one preferable embodiment of the invention, the 5′ end of A₀ or an internal site on the 5′ side of the priming region is rendered resistant to exonucleolysis. By this means and after, or simultaneously with, the pyrophosphorolysis step, an exonuclease having 5′-3′ exonucleolytic activity can optionally be added to the reaction medium for the purpose of digesting any other nucleic acid molecules present whilst leaving A₀ and any material comprising the partially digested strand A₁ intact. Suitably, this resistance to exonucleolysis is achieved by introducing one or more blocking groups into the oligonucleotide A₀ at the required point. In some embodiments, these blocking groups may be selected from phosphorothioate linkages and other backbone modifications commonly used in the art, C3 spacers, phosphate groups, modified bases and the like.

In some embodiments, the identification region will comprise or have embedded within a barcoding region which has a unique sequence and is adapted to be indirectly identified using a sequence-specific molecular probe applied to the amplified components A₂ or directly by the sequencing of these components. Examples of molecular probes which may be used include, but are not limited to, molecular beacons, TaqMan® probes, Scorpion® probes and the like.

In some embodiments the A₂ strand or a desired region thereof is caused to undergo amplification so that multiple, typically many millions, of copies are made. This is achieved by priming a region of A₂ and subsequently any amplicons derived therefrom with single-stranded primer oligonucleotides, provided for example in the form of a forward/reverse or sense/antisense pair, which can anneal to a complementary region thereon. The primed strand then becomes the point of origin for amplification. Amplification methods include, but are not limited to, thermal cycling and isothermal methods such as the polymerase chain reaction, recombinase polymerase amplification and rolling circle amplification; the last of these being applicable when A₂ is circularised. By any of these means, many amplicon copies of a region of A₂ and in some instances its sequence complement can be rapidly created. The exact methodologies for performing any of these amplification methods will be well-known to one of ordinary skill and the exact conditions and temperature regimes employed are readily available in the general literature to which the reader is directed. Specifically, in the case of the polymerase chain reaction (PCR), the methodology generally comprises extending the primer oligonucleotide against the A₂ strand in the 5′-3′ direction using a polymerase and a source of the various single nucleoside triphosphates until a complementary strand is produced; dehybridising the double-stranded product produced to regenerate the A₂ strand and the complementary strand; re-priming the A₂ strand and any of its amplicons and thereafter repeating these extension/dehybridisation/repriming steps multiple times to build-up a concentration of A₂ amplicons to a level where they can be reliably detected.

In some embodiments, the second reaction mixture further comprises a phosphatase or phosphohydrolase to remove by hydrolysis the nucleoside triphosphates produced by the pyrophosphorolysis reaction thereby ensuring that the pyrophosphorolysis reaction can continue and does not become out-competed by the forward polymerisation reaction.

In some embodiments, prior to or during step (v) the products of the previous step are treated with a pyrophosphatase to hydrolyse the pyrophosphate ion, preventing further pyrophosphorolysis from occurring and favouring the forward polymerisation reaction. In some embodiments, prior to or during step (v) the products of the previous step are treated with an exonuclease.

In some embodiments, the enzyme which performs pyrophosphorolysis of A₀ to form partially digested strand A₁ also amplifies A₂. The person skilled in the art will appreciate there exist many such enzymes.

The oligonucleotides A₂ are detected and the information obtained is used to infer whether the polynucleotide target sequence is present or absent in the original analyte and/or a property associated therewith. For example, by this means a target sequence characteristic of a cancerous tumour cell may be detected with reference to specific SNPs being looked for. As a further example, a target sequence characteristic of a cancerous tumour cell may be detected with reference to specific methylation sites being looked for.

In another embodiment, a target sequence characteristic of the genome of a virus of bacterium (including new mutations thereof) may be detected. Many methods of detecting amplicons or identification regions of A₂ can be employed including for example an oligonucleotide binding dye, a sequence-specific molecular probe such as fluorescently-labelled molecular beacon or hairpin probe. Alternatively, direct sequencing of A₂ of the amplicons thereof can be performed using one of the direct sequencing methods employed or reported in the art. Where oligonucleotide binding dyes, fluorescently labelled beacons or probes are employed it is convenient to detect the resulting signal using an arrangement comprising a source of stimulating electromagnetic radiation (laser, LED, lamp etc.) and a photodetector arranged to detect emitted fluorescent light and to generate therefrom a signal comprising a data stream which can be analysed by a microprocessor or a computer using specifically-designed algorithms.

In some embodiments, detection is achieved using one or more oligonucleotide fluorescent binding dyes or molecular probes. In such embodiments, an increase in signal over time resulting from the generation of amplicons of A₂ is used to infer the concentration of the target sequence in the analyte. In some embodiments that the final step of the method further comprises the steps of:
i. labelling the products of step (iv) using one or more oligonucleotide fluorescent binding dyes or molecular probes;
ii. measuring the fluorescent signal of the products;
iii. exposing the products to a set of denaturing conditions; and

identifying the polynucleotide target sequence in the analyte by monitoring changes in the fluorescent signal of the products during exposure to the denaturing conditions.

In another aspect of the invention, there is provided a method of identifying a target polynucleotide sequence in a given nucleic acid analyte characterised by the steps of any previous embodiment of the invention wherein the multiple copies of A₂, or a region of A₂, are labelled using one or more oligonucleotide fluorescent binding dyes or molecular probes. The fluorescent signal of these multiple copies is measured and the multiple copies are exposed to a set of denaturing conditions. The target polynucleotide sequence is then identified by monitoring a change in the fluorescent signal of the multiple copies during exposure to the denaturing conditions.

In some embodiments, the denaturing conditions may be provided by varying the temperature e.g. increasing the temperature to a point where the double strand begins to dissociate. Additionally or alternatively, the denaturing conditions may also be provided by varying the pH such that the conditions are acidic or alkaline, or by adding in additives or agents such as a strong acid or base, a concentrated inorganic salt or organic solvent e.g. alcohol.

In another aspect of the invention, there is provided the use of the methods described above to screen mammalian subjects, especially human patients, for the presence of infectious diseases, cancer or for the purpose of generating companion diagnostic information.

In a further aspect of the invention, there is provided control probes for use in the methods as described above. Embodiments of the current invention include those wherein the presence of a specific target sequence, or sequences, is elucidated by the generation of a fluorescent signal. In such embodiments, there may inevitably be a level of signal generated from non-target DNA present in the sample. For a given sample, this background signal has a later onset than the ‘true’ signal, but this onset may vary between samples. Accurate detection of the presence of low concentrations of target sequence, or sequences, thus relies on knowledge of what signal is expected in its absence. For contrived samples references are available, but for true ‘blind’ samples from patients this is not the case. The control probes (E₀) are utilised to determine the expected background signal profile for each assay probe. The control probe targets a sequence not expected to be present in the sample and the signal generated from this probe can then be used to infer the expected rate of signal generation from the sample in the absence of target sequence.

Thus there is provided a method of detecting a target polynucleotide sequence in a given nucleic acid analyte according to any of the previously described methods characterised by the steps of:
(a) either subsequently or concurrently repeating the steps of the methods using a single-stranded probe oligonucleotide E₀ having a 3′ end region at least partially mismatched to the target sequence, either using a separate aliquot of the sample or in the same aliquot and using a second detection channel;
(b) inferring the background signal expected to be generated from the molecular system comprising A₀ in the absence of any target analyte in the sample; and
(c) through comparison of the expected background signal inferred in (a) with the actual signal observed in the presence of the target analyte inferring the presence or absence of the polynucleotide target sequence in the analyte.

In some embodiments, the control probe (E₀) and the molecular system comprising A₀ are added to separate portions of the sample while in another embodiment the E₀ and the molecular system comprising A₀ are added to the same portion of the sample and different detection channels (e.g. different colour dyes) used to measure their respective signals. The signal generated by E₀ may then be utilised to infer and correct for the background signal expected to be generated by the molecular system comprising A₀ in the absence of the polynucleotide target sequence in the sample. For example, a correction of the background signal may involve the subtraction of the signal observed from E₀ from that observed from the molecular system comprising A₀, or through the calibration of the signal observed from the molecular system comprising A₀ using a calibration curve of the relative signals generated by the molecular system comprising A₀ and E₀ under varying conditions.

In some embodiments, a single E₀ can be used to calibrate all of the assay probes which may be produced.

In some embodiments, a separate E₀ may be used to calibrate each amplicon of the sample DNA generated in an initial amplification step. Each amplicon may contain multiple mutations/target sequences of interest, but a single E₀ will be sufficient to calibrate all of the assay probes against a single amplicon.

In a further embodiment, a separate E₀ may be used for each target sequence. For example, if a C>T mutation is being targeted, an E₀ could be designed that targets a C>G mutation in the same site that is not known to occur in patients. The signal profile generated by E₀ under various conditions can be assessed in calibration reactions and these data used to infer the signal expected from the assay probe targeting the C>T variant when this variant is not present.

The specificity of the methods of the current invention may be improved by the introduction of blocking oligonucleotides. For example a blocking oligo nucleotide can be introduced so as to hybridise to at least a portion of wild-type DNA, promoting annealing of the molecular system comprising A₀ only to the target polynucleotide sequences and not the wildtype. Alternatively or additionally, blocking oligonucleotides can be used to improve the specificity of the polymerase chain reaction (PCR) to prevent amplification of any wild type sequence present. The general technique used is to design an oligonucleotide that anneals between the PCR primers and is not able to be displaced or digested by the PCR polymerase. The oligonucleotide is designed to anneal to the non-target (usually healthy) sequence, while being mismatched (often by a single base) to the target (mutant) sequence. This mismatch results in a different melting temperature against the two sequences, the oligonucleotide being designed to remain annealed to the non-target sequence at the PCR extension temperature while dissociating from the target sequence.

The blocking oligonucleotides may often have modifications to prevent its digestion by the exonuclease activity of the PCR polymerase, or to enhance the melting temperature differential between the target and non-target sequence.

The incorporation of a locked nucleic acid (LNA) or other melting temperature altering modification into a blocking oligonucleotide can significantly increase the differential in melting temperature of the oligonucleotide against target and non-target sequences.

Thus there is provided an embodiment of the invention wherein blocking oligonucleotides are used. The blocking oligonucleotides, in some embodiments, must be resistant to the pyrophosphorolysing (PPL) reaction to ensure they are not digested or displaced. This can be achieved in a number of different ways, for example via mismatches at their 3′ ends or through modifications such as phosphorothioate bonds or spacers.

In such embodiments or an aspect of the present invention where blocking oligonucleotides are used, the method of detecting a target polynucleotide sequence in a given nucleic acid analyte is characterised by annealing single-stranded blocking oligonucleotides to at least a subset of non-target polynucleotide sequences before, or during, the same step wherein the analyte target sequence is annealed to a molecular system comprising a probe oligonucleotide A₀ to create a first intermediate product which is at least partially double-stranded and in which the 3′ end of A₀ forms a double-stranded complex with the analyte target sequence.

In some embodiments, the blocking oligonucleotides are made to be resistant to the pyrophosphorolysing reaction via mismatches at their 3′ ends. In another embodiment, the blocking oligonucleotides are made to be resistant via the presence of a 3′-blocking group. In another embodiment the blocking oligonucleotides are made to be resistant via the presence of spacers or other internal modifications. In a further embodiment the blocking oligonucleotides include both a melting temperature increasing modification or modified nucleotide base and are rendered resistant to pyrophosphorolysis.

In an aspect of the present invention, there is provided a method for the detection of a polynucleotide target sequence in a given nucleic acid analyte in a sample, comprising the steps of:
(a) introducing a blocking oligonucleotide to a first reaction mixture comprising one or more nucleic acid analytes, wherein the blocking oligonucleotide anneals to at least a subset of non-target polynucleotide sequences;
(b) introducing the mixture produced in (a) to a second reaction comprising a molecular system, which may be as previously or subsequently described;
(c) treating A₀ with an enzyme for pyrophosphorolysis thereby removing complementary nucleotides from the 3′-end of the A₀ that is fully hybridised to the target, to form shortened probe A₁;
(d) using C to displace the 3′-end of A₁ from the target;
(e) ligating the ends of the A₁ to form a circle using the pre-hybridised C, to form circular A₂; and
(f) detecting the presence of A₂.

In some embodiments, the first reaction mixture further comprises one or more primers, deoxynucleotide triphosphates (dNTP) and an amplification enzyme and during step (a) the nucleic acid analytes present in a sample undergo amplification and wherein after amplification of the given nucleic acid analytes and prior to (b), the sample is further treated with a proteinase.

In some embodiments, prior to step (a), nucleic acid analytes present in a sample are amplified and after amplification of the given nucleic acid analytes the sample is further treated with a proteinase.

In some embodiments, the sample is treated with a proteinase prior to step (a). In some embodiments, the sample is treated with a proteinase during step (a). In some embodiments, the sample is treated with a proteinase after step (a).

In some embodiments, the first and second reaction mixtures are combined such that the method comprises the steps of:
(a) introducing one or more nucleic acid analytes to a combined reaction mixture comprising:
   i. a molecular system;
   ii. a blocking oligonucleotide;
   iii. a pyrophosphorolysing enzyme; and
   iv. a ligase;

wherein the blocking oligonucleotide anneals to at least a subset of non-target polynucleotide sequences and wherein the target analyte anneals to the molecular system comprising a probe oligonucleotide A₀ portion of the molecular system to create a first intermediate product which is at least partially double-stranded and in which the 3′ end of A₀ forms a double-stranded complex and A₀ is pyrophosphorolysed in the 3′-5′ direction from the 3′ end to create at least a partially digested strand A₁ and A₁ undergoes ligation to form A₂;
(b) detecting a signal derived from the products of the previous step, wherein the products are A₂ or a portion thereof, or multiple copies of A₂ or multiple copies of a portion thereof, and inferring therefrom the presence or absence of the polynucleotide target sequence in the analyte.

In some embodiments of the methods, the blocking oligonucleotide is perfectly complementary to a target nucleic acid analyte and mismatched to non-target nucleic acid analytes such that:
the non-target nucleic acid analyte anneals imperfectly to the blocking oligonucleotide to form an intermediate product which cannot be digested by pyrophosphorolysis to the extent needed for it to melt from the non-target molecule;
the target nucleic acid analyte anneals perfectly to the blocking oligonucleotide to form an intermediate product which is at least partially double-stranded at the 3′ end of the blocking oligonucleotide and the blocking oligonucleotide is pyrophosphorolysed in the 3′-5′ direction, releasing the target nucleic acid analyte;
the target nucleic acid analyte anneals to the molecular system comprising a probe oligonucleotide A₀ portion of the molecular system to create a first intermediate product which is at least partially double-stranded and in which the 3′ end of A₀ forms a double-stranded complex and A₀ is pyrophosphorolysed in the 3′-5′ direction from the 3′ end to create at least a partially digested strand A₁ and A₁ undergoes ligation to form A₂; and a signal derived from the products of the previous step is detected, wherein the products are A₂ or a portion thereof, or multiple copies of A₂ or multiple copies of a portion thereof, and inferring therefrom the presence or absence of the polynucleotide target sequence in the analyte.

In some embodiments, the blocking oligonucleotide comprises a modification to render it resistant to digestion by exonucleolysis or pyrophosphorolysis.

In some embodiments, the blocking oligonucleotide comprises a 3′ modification to render it resistant to digestion by exonucleolysis or pyrophosphorolysis.

In some embodiments, the blocking oligonucleotide comprises a 5′ modification to render it resistant to digestion by exonucleolysis.

References herein to ‘phosphatase enzymes’ refer to any enzymes, or functional fragments thereof, with the ability to remove by hydrolysis the nucleoside triphosphates produced by the methods of the current invention. This includes any enzymes, or functional fragments thereof, with the ability to cleave a phosphoric acid monoester into a phosphate ion and an alcohol.

References herein to ‘pyrophosphatase enzymes’ refer to any enzymes, or functional fragments thereof, with the ability to catalyse the conversion of one ion of pyrophosphate to two phosphate ions.

This also includes inorganic pyrophosphatases and inorganic diphosphatases. A non-limiting example is thermostable inorganic pyrophosphate (TIPP).

In some embodiments there is provided a modified version of any previously described embodiment wherein the use of a pyrophosphatase is optional.

In some embodiments of the invention there is provided a kit for use in a method of detecting a target polynucleotide sequence in a given nucleic acid analyte present in a sample, comprising:
(a) a molecular system comprising a probe oligonucleotide A₀, capable of forming a first intermediate product with a target polynucleotide sequence, said intermediate product being at least partially double-stranded;
(b) a ligase;
(c) a pyrophosphorolysing enzyme capable of digesting the first intermediate product in the 3′-5′ direction from the end of A₀ to create a partially digested strand A₁;
(d) suitable buffers.

In one embodiment, the 3′ end of A₀ is complementary to the target sequence.

In one embodiment, the 3′ end of A₀ is perfectly complementary to the target sequence.

In one embodiment, the kit further comprises at least one single-stranded primer oligonucleotide that is substantially complementary to a portion of A₀.

In one embodiment, the kit further comprises an amplification enzyme.

In one embodiment, the kit further comprises one or more blocking oligonucleotides.

In one embodiment, the kit further comprises one or more primers wherein one or more of the primers has a non-complementary 5′ tail.

In one embodiment, one or more of the primers has a 5′ phosphate.

In one embodiment, one or more of the primers is 5′ protected.

In one embodiment, the 3′ end of A₀ is perfectly complementary to the target polynucleotide sequence.

In one embodiment, the ligase is substantially lacking in single-strand ligation activity.

In some embodiments, the kit may alternatively further comprise:
two or more Ligation Chain Reaction (LCR) probe oligonucleotides that are complementary to adjacent sequences on A₁ wherein when the probes are successfully annealed the 5′ phosphate of one LCR probe is directly adjacent to the 3′OH of the other LCR probe; and
one or more ligases.

In some embodiments, in the presence of A₂ the two LCR probes will successfully anneal to A₂ and be ligated together to form one oligonucleotide molecule which subsequently acts as a new target for second-round covalent ligation, leading to geometric amplification of the target of interest, in this case A₂. The ligated products, or amplicons, are complementary to A₂ and function as targets in the next cycle of amplification. Thus, exponential amplification of the specific target DNA sequences is achieved through repeated cycles of denaturation, hybridization, and ligation in the presence of excess LCR probes. From this, the presence of A₂ and hence the target polynucleotide sequence is inferred.

In some embodiments, in the presence of A₂ the two PCR probes will successfully anneal to A₂ and be ligated together to form one oligonucleotide molecule which then acts as a new target for second-round covalent ligation, leading to geometric amplification of the target of interest, in this case A₂, which is then detected.

In some embodiments, the kit may further comprise:
A hairpin oligonucleotide 1 (HO1) comprising a fluorophore-quencher pair, wherein HO1 is complementary to A₂ and when annealed to A₂ the hairpin structure of HO1 opens and the fluorophore-quencher pair separate; and
A hairpin oligonucleotide 2 (HO2) comprising a fluorophore-quencher pair, wherein HO2 is complementary to the open HO1 and when annealed to HO1 the hairpin structure of HO2 opens and the fluorophore-quencher pair separate.

In some embodiments, the kit may further comprise a plurality of HO1 and HO2.

In some embodiments, the kit may alternatively further comprise an oligonucleotide A comprising a substrate arm, a partial catalytic core and a sensor arm;
an oligonucleotide B comprising a substrate arm, a partial catalytic core and a sensor arm; and
a substrate comprising a fluorophore quencher pair;

wherein the sensor arms of oligonucleotides A and B are complementary to flanking regions of A₂ such that in the presence of A₂, oligonucleotides A and B are combined to form a catalytically, multicomponent nucleic acid enzyme (MNAzyme).

In some embodiments, the kit may alternatively further comprise a partially double-stranded nucleic acid construct wherein:
one strand comprises at least one RNA base, at least one fluorophore and wherein a region of this strand is complementary to a region of A₂ and wherein this strand may be referred to as the ‘substrate’ strand; and
the other stand comprises at least one quencher and wherein a region of this strand is complementary to a region of A₂ adjacent to that which the substrate strand is complementary to, such that in the presence of A₂ the partially double stranded nucleic acid construct becomes substantially more double-stranded.

In other words, the partially double stranded nucleic acid construct, in the presence of A₂, has a double-stranded portion which is greater in size.

In some embodiments, the kit may further comprise an enzyme for removal of the at least one RNA base.

In some embodiments, the enzyme is Uracil-DNA Glycosylase (UDG) and the RNA base is uracil.

In some embodiments, the kit may alternatively further comprise:
an oligonucleotide complementary to a region of A₂ including the site of ligation, comprising one or multiple fluorophores arranged such that their fluorescence is quenched either by their proximity to each other or to one or more fluorescence quenchers;
a double strand specific DNA digestion enzyme;

wherein, in the presence of A₂, the labelled oligonucleotide is digested such that the fluorophores are separated from each other or from their corresponding quenchers, and a fluorescent signal, and hence the presence of A₂, is detectable.

In some embodiments, the double strand specific DNA digestion enzyme is an exonuclease.

In some embodiments, the double strand specific DNA digestion enzyme is a polymerase with proofreading activity.

In some embodiments, the fluorophore of the kit may be selected from dyes of the fluorescein family, the carboxyrhodamine family, the cyanine family, the rhodamine family, polyhalofluorescein-family dyes, hexachlorofluorescein-family dyes, coumarin-family dyes, oxazine-family dyes, thiazine-family dyes, squaraine-family dyes, and chelated lanthanide-family dyes.

In some embodiments, the fluorophore of the kit may be selected from any of the commercially available dyes.

In some embodiments, the quencher of the kit may be selected from those available those available under the trade designations Black Hole™, Eclipse™. Dark, Qx1J, and Iowa Black™.

In some embodiments, the quencher of the kit may be selected from any of the commercially available quenchers.

In some embodiments, the kit may further comprise one or more partially double stranded DNA constructs wherein each construct contains one or more fluorophores and one or more quenchers. In some embodiments, when the construct is partially double-stranded the one or more fluorophores and one or more quenchers are located in close enough proximity to each other such that sufficient quenching of the one or more fluorophores occurs.

In some embodiments, the construct is one strand of DNA with a self-complementary region that is looped back on itself.

In some embodiments, the construct comprises one primer of a primer pair.

In some embodiments, the kit may further comprise the other primer of a primer pair.

In some embodiments, a portion of the single stranded section of the construct hybridises to A₂ and is extended against it by a DNA polymerase. In some embodiments, the other primer of the primer pair then hybridises to the extended construct. This primer is then extended against the construct, displacing the self-complementary region. Thus, the one or more fluorophores and one or more dyes are separated sufficiently for a fluorescent signal to be detected, indicating the presence of A₂. In such an embodiment the construct may be known as a Sunrise Primer.

In some embodiments, the construct comprises two separate DNA strands.

In some embodiments, a portion of the single stranded section of the construct hybridises to A₂ and is extended against it by a DNA polymerase. In some embodiments, the other primer of the primer pair then hybridises to the extended construct. This primer is then extended against the construct, in the direction of the double stranded section, displacing the shorter of the DNA strands and thus the one or more fluorophores and one or more dyes are separated sufficiently for a fluorescent signal to be detected, indicating the presence of A₂. In such an embodiment the construct may be known as a Molecular Zipper.

The person skilled in the art will appreciate that for both the Sunrise Primer and Molecular Zipper it is possible for the one or more fluorophores and the one or more quencher pairs to be located at various positions within each respective construct. The key feature is that each pair is located in sufficient proximity to one another that in the absence of A₂, i.e. when no extension and strand displacement has occurred, no fluorescent signal is emitted.

In one embodiment the kit further comprises a source of pyrophosphate ion. Suitable source(s) of pyrophosphate ion are as described previously.

In some embodiments, the kit further comprises suitable positive and negative controls. In some embodiments, the kit may further comprise one or more control probes (E₀) which are as previously described. In some embodiments, the kit may further comprise one or more control probes (Eₒ) and one or more blocking oligonucleotides.

In some embodiments, the 5′ end of A₀ may be rendered resistant to 5′-3′ exonuclease digestion and the kit may further comprise a 5′-3′ exonuclease. In some embodiments C may comprise a 3′ or internal modification protecting it from 3′-5′ exonuclease digestion.

In some embodiments, the kit may further comprise dNTPs, a polymerase, primers and suitable buffers for the initial amplification of a target polynucleotide sequence present in a sample. In some such embodiments at least one primer used for such initial amplification comprises a 5′ or internal blocking modification and the kit further comprises a 5′-3′ exonuclease. In some such embodiments at least one other primer comprises a 5′ phosphate group.

In some embodiments, the kit may further comprise a dUTP incorporating high fidelity polymerase, dUTPs and uracil-DNA N-glycosylase (UDG).

In some embodiments, the kit may further comprise a phosphatase or a phosphohydrolase. In some embodiments, the kit may further comprise a pyrophosphatase. The pyrophosphatase may be hot start.

In some embodiments, the kit may further comprise a proteinase.

In some embodiments, the kit may further comprise one or more oligonucleotide binding dyes or molecular probes.

In some embodiments, the kit may further comprise multiple molecular systems comprising A₀, each selective for a different target sequence and each including an identification region.

In some embodiments, the kit may further comprise an enzyme for the formation of DNA from an RNA template.

In some embodiments, the enzyme is a reverse transcriptase.

In some embodiments, the one or more enzymes of the kit may be hot start.

In some embodiments, the one or more enzymes of the kit may be thermostable.

In some embodiments, the kit may further comprise suitable washing and buffer reagents.

In some embodiments, the amplification enzyme, of (e), and the pyrophosphorolysing enzyme are the same.

In some embodiments, the amplification enzyme and the pyrophosphorolysis enzyme are the same.

The kit may further comprise purification devices and reagents for isolating and/or purifying a portion of polynucleotides, following treatment as described herein. Suitable reagents are well known in the art and include gel filtration columns and washing buffers.

In some embodiments, the kit further comprises at least one single-stranded primer oligonucleotide that is substantially complementary to a portion of A₀, an amplification enzyme and dNTPs.

In some embodiments, the kit further comprises one or more oligonucleotide binding dyes or molecular probes.

In some embodiments, the kit further comprises multiple molecular systems, each comprising a A₀, each selective for a different target sequence and each including an identification region.

In some embodiments, the kit further comprises:
two or more Ligation Chain Reaction (LCR) probe oligonucleotides that are complementary to adjacent sequences on A₁ wherein when the probes are successfully annealed the 5′ phosphate of one LCR probe is directly adjacent to the 3′OH of the other LCR probe; and
one or more ligases.

In some embodiments, the kit further comprises one or more polymerases.

In some embodiments of the kit, the one or more polymerases are the same as the pyrophosphorolysis enzyme.

In some embodiments, the kit further comprises:
A hairpin oligonucleotide 1 (HO1) comprising a fluorophore-quencher pair, wherein HO1 is complementary to A₂ and when annealed to A₂ the hairpin structure of HO1 opens and the fluorophore-quencher pair separate; and
A hairpin oligonucleotide 2 (HO2) comprising a fluorophore-quencher pair, wherein HO2 is complementary to the open HO1 and when annealed to HO1 the hairpin structure of HO2 opens and the fluorophore-quencher pair separate.

In some embodiments, the kit further comprises a plurality of HO1 and HO2.

In some embodiments, the kit further comprises the kit further comprises:
an oligonucleotide A comprising a substrate arm, a partial catalytic core and a sensor arm;
an oligonucleotide B comprising a substrate arm, a partial catalytic core and a sensor arm;
and
a substrate comprising a fluorophore quencher pair;

wherein the sensor arms of oligonucleotides A and B are complementary to flanking regions of A₂ such that in the presence of A₂, oligonucleotides A and B are combined to form a catalytically, multicomponent nucleic acid enzyme (MNAzyme).

In some embodiments, the kit further comprises a partially double-stranded nucleic acid construct wherein:
one strand comprises at least one RNA base, at least one fluorophore and wherein a region of this strand is complementary to a region of A₂ and wherein this strand may be referred to as the ‘substrate’ strand; and
the other stand comprises at least one quencher and wherein a region of this strand is complementary to a region of A₂ adjacent to that which the substrate strand is complementary to, such that in the presence of A₂ the partially stranded nucleic acid construct becomes substantially more double-stranded.

In some embodiments, the kit further comprises an enzyme for removal of the at least one RNA base.

In some embodiments of the kit, the enzyme is Uracil-DNA Glycosylase (UDG) and the RNA base is uracil.

In some embodiments, the kit further comprises:
an oligonucleotide complementary to a region of A₂ including the site of ligation, comprising one or multiple fluorophores arranged such that their fluorescence is quenched either by their proximity to each other or to one or more fluorescence quenchers;
a double strand specific DNA digestion enzyme;

wherein, in the presence of A₂, the labelled oligonucleotide is digested such that the fluorophores are separated from each other or from their corresponding quenchers, and a fluorescent signal, and hence the presence of A₂, is detectable.

In some embodiments of the kit, the double strand specific DNA digestion enzyme is an exonuclease.

In some embodiments of the kit, the double strand specific DNA digestion enzyme is a polymerase with proofreading activity.

In some embodiments of the kit, the fluorophore is selected from dyes of the fluorescein family, the carboxyrhodamine family, the cyanine family, the rhodamine family, polyhalofluorescein-family dyes, hexachlorofluorescein-family dyes, coumarin-family dyes, oxazine-family dyes, thiazine-family dyes, squaraine-family dyes, and chelated lanthanide-family dyes.

In some embodiments of the kit, the quencher is selected from those available under the trade designations Black Hole™, Eclipse™. Dark, Qx1J, and Iowa Black™.

In some embodiments, the kit further comprises a phosphatase or a phosphohydrolase.

In some embodiments, the kit further comprises a pyrophosphatase.

In some embodiments, the kit further comprises an enzyme for the formation of DNA from an RNA template.

In some embodiments of the kit, the enzyme is a reverse transcriptase.

In some embodiments of the kit, one or more enzymes are hot start.

In some embodiments of the kit, one or more enzymes are thermostable.

In some embodiments, the kit may further comprise suitable washing and buffer reagents.

In some embodiments, the kit further comprises an epigenetic-sensitive and/or an epigenetic-dependent restriction enzyme, which may be as previously described.

In some embodiments, the kit further comprises a methylation-sensitive and/or methylation-dependent restriction enzyme.

In some embodiments, the kit further comprises one or methyl-CpG binding domain (MBD) proteins.

In some embodiments, the kit further comprises one or more 5-methylcytidine (5-mC) antibodies.

In some embodiments, the kit further comprises one or more of MBD2b protein and/or one or more of the MBD2b/MBD3L1 complex.

In some embodiments, the kit further comprises reagents suitable for methylation-specific multiplex ligation-dependent probe amplification (MS-MLPA).

In one embodiment of the invention there is provided a device comprising:
at least a fluid pathway between a first region, a second region and a third region, wherein the first region comprises one or more wells, wherein each well comprises: dNTPs;
at least one single-stranded primer oligonucleotide;
an amplification enzyme for the initial amplification of DNA present in a sample; and

wherein the second region comprises one or more wells, wherein each well comprises:
a molecular system comprising probe oligonucleotide A₀, capable of forming a first intermediate product with a target polynucleotide sequence, said intermediate product being at least partially double-stranded;
a pyrophosphorolysing enzyme capable of digesting the first intermediate product in the 3′-5′ direction from the end of A₀ to create a partially digested strand A₁; and

wherein the third region comprises one or more wells, wherein each well comprises: dNTPs;
buffers;
an amplification enzyme;
a means for detecting a signal derived from A₂ or a portion thereof, or multiple copies of A₂ or multiple copies of a portion thereof; and

wherein the wells of the second region or the wells of the third region further comprise at least one single-stranded primer oligonucleotide that is substantially complementary to a portion of A₀.

In some embodiments, one or more wells of the first region comprise one or more blocking oligonucleotides, as previously or subsequently described.

In some embodiments, one or more wells of the second region comprises one or more blocking oligonucleotides, as previously or subsequently described.

In some embodiments, the wells of the first region comprise:
dNTPs;
one or more single-stranded primer oligonucleotides;
an amplification enzyme for the initial amplification of DNA present in a sample;

wherein one or more of the primers has a non-complimentary 5′ tail.

In some embodiments, one or more of the primers has a 5′ phosphate.

In some embodiment, one or more of the primers is 5′ protected.

In some embodiments, a means for detecting a signal is located within one or more wells of the third region.

In some embodiments, a means for detecting a signal is located within the third region of the device.

In some embodiments, a means for detecting a signal is located within an adjacent region of the device.

In some embodiments, the dNTPs of each well of the first region may be dUTP, dGTP, dATP and dCTP and each well may further comprise a dUTP incorporating high fidelity polymerase and uracil-DNA N-glycosylase (UDG).

In some embodiments, the dNTPs of each well of the third region may be dUTP, dGTP, dATP and dCTP and each well may further comprise a dUTP incorporating high fidelity polymerase and uracil-DNA N-glycosylase (UDG).

In some embodiments, each well of the second region may further comprise a source of pyrophosphate ion.

In some embodiments, the 5′ end of A₀ may be rendered resistant to 5′-3′ exonuclease digestion and the wells of the second region may further comprises a 5′-3′ exonuclease.

In some embodiments, the dNTPs may be hot start and each well of the second region may further comprise a phosphatase or a phosphohydrolase.

In some embodiments, each well of the second region may further comprise a pyrophosphatase.

In some embodiments, the pyrophosphatase may be a hot start.

In some embodiments, each well of the third region may further comprise one or more oligonucleotide binding dyes or molecular probes.

In some embodiments, each well of the second region may comprise at least one or more different molecular systems comprising A₀ that is selective for a target sequence including an identification region.

In some embodiments, the amplification enzyme and the pyrophosphorolysing enzyme in the second region may be the same.

In some embodiments, there may be a fourth region comprising one or more wells, wherein each well may comprise a proteinase and wherein said fourth region may be located between the first and second regions.

In some embodiments, the second and third regions of the device may be combined such that the wells of the second region further comprise:
dNTPs;
buffers;
an amplification enzyme; and
a means for detecting a signal derived from A₁ or a portion thereof, or multiple copies of A₁ or multiple copies of a portion thereof.

The wells of the second region may further comprise one or more blocking oligonucleotides as previously or subsequently described.

In some embodiments, a means for detecting a signal is located within one or more wells of the second region.

In some embodiments, a means for detecting a signal is located within the second region of the device.

In some embodiments, a means for detecting a signal is located within an adjacent region of the device.

In some embodiments, there is provided a device comprising:
a fluid pathway between a first region and second region, wherein the first region comprises one or more wells, wherein one or more well comprises:
   a molecular system comprising a probe oligonucleotide A₀, which is capable of forming a first intermediate product with a target polynucleotide sequence, said intermediate product being at least partially double-stranded;
   a pyrophosphorolysing enzyme capable of digesting the first intermediate product in the 3′-5′ direction from the end of A₀ to create a partially digested strand A₁; and
   one or more ligases capable of ligating A₁ to create an oligonucleotide A₂.
wherein the second region comprises one or more wells.

The wells of the first region may further comprise one or more blocking oligonucleotides as previously or subsequently described.

In some embodiments, one or more well of the first region may further comprise a source of ions to drive the pyrophosphorolysis reaction forward.

In some embodiments, the ions are pyrophosphate ions.

In some embodiments, the 5′ end of A₀ is resistant to 5′-3′ exonuclease digestion and wherein the wells of the first region further comprise a 5′-3′ exonuclease.

In some embodiments, the device may further comprise a third region comprising one or more wells which is joined to the first region by a fluid pathway and wherein one or more wells of the third region comprises:
dNTPs;
a single-stranded primer oligonucleotide; and
an amplification enzyme.

The wells of the third region may further comprise one or more blocking oligonucleotides as previously or subsequently described.

In some embodiments, the dNTPs of the third region may be dUTP, dGTP, dCTP and dATP; the amplification enzyme may be a dUTP incorporating high fidelity polymerase; and one or more wells of the third region may further comprise uracil-DNA N-glycosylase.

In some embodiments, the device may further comprise a fourth region, located between the first and third regions, comprising one or more wells, wherein one or more wells may comprise a proteinase.

In some embodiments, the one or more wells of the first or second regions may further comprise a ligase

In some embodiments, one or more wells of the first region may comprise at least one or more different molecular systems comprising A₀ each selective for a different target sequence and each including an identification region.

In some embodiments, the wells of the second region may comprise:
dNTPs;
buffers;
an amplification enzyme;
a means for detecting a signal derived from A₁ or a portion thereof, or multiple copies of A₁ or multiple copies of a portion thereof.

The wells of the second region may further comprise one or more blocking oligonucleotides as previously or subsequently described.

Embodiments of the invention may comprise one or more blocking oligonucleotides located in one or more regions which comprise dNTPs; buffers; amplification enzymes etc.

In some embodiments, a means for detecting a signal is located within one or more wells of the second region.

In some embodiments, a means for detecting a signal is located within the second region of the device.

In some embodiments, a means for detecting a signal is located within an adjacent region of the device.

In some embodiments, one or more wells of the second region may further comprise one or more oligonucleotide binding dyes or molecular probes.

In some embodiments, the amplification enzyme and the pyrophosphorolysing enzyme of the device are the same.

In some embodiments, the wells of the second region further comprise:
two or more Ligation Chain Reaction (LCR) probe oligonucleotides that are complementary to adjacent sequences on A₁ wherein when the probes are successfully annealed the 5′ phosphate of one LCR probe is directly adjacent to the 3′OH of the other LCR probe; and
one or more ligases.

In some embodiments, the wells of the second region may further comprise:
A hairpin oligonucleotide 1 (HO1) comprising a fluorophore-quencher pair, wherein HO1 is complementary to A₂ and when annealed to A₂ the hairpin structure of HO1 opens and the fluorophore-quencher pair separate; and
A hairpin oligonucleotide 2 (HO2) comprising a fluorophore-quencher pair, wherein HO2 is complementary to the open HO1 and when annealed to HO1 the hairpin structure of HO2 opens and the fluorophore-quencher pair separate.

In some embodiments, the wells of the second region may further comprise a plurality of HO1 and HO2.

In some embodiments, the wells of the second region may further comprise:
an oligonucleotide A comprising a substrate arm, a partial catalytic core and a sensor arm;
an oligonucleotide B comprising a substrate arm, a partial catalytic core and a sensor arm; and
a substrate comprising a fluorophore quencher pair;

wherein the sensor arms of oligonucleotides A and B are complementary to flanking regions of A₂ such that in the presence of A₂, oligonucleotides A and B are combined to form a catalytically, multicomponent nucleic acid enzyme (MNAzyme).

In some embodiments, the wells of the second region may comprise a partially double-stranded nucleic acid construct wherein:
one strand comprises at least one RNA base, at least one fluorophore and wherein a region of this strand is complementary to a region of A₂ and wherein this strand may be referred to as the ‘substrate’ strand; and
the other stand comprises at least one quencher and wherein a region of this strand is complementary to a region of A₂ adjacent to that which the substrate strand is complementary to, such that in the presence of A₂ the partially stranded nucleic acid construct becomes substantially more double-stranded.

In some embodiments, the wells of the second region may further comprise an enzyme for the removal of the at least one RNA base.

In some embodiments, the enzyme is Uracil-DNA Glycosylase (UDG) and the RNA base is uracil.

In some embodiments, one or more wells of the second region may further comprise:
an oligonucleotide complementary to a region of A₂ including the site of ligation, comprising one or multiple fluorophores arranged such that their fluorescence is quenched either by their proximity to each other or to one or more fluorescence quenchers;
a double strand specific DNA digestion enzyme;

wherein, in the presence of A₂, the labelled oligonucleotide is digested such that the fluorophores are separated from each other or from their corresponding quenchers, and a fluorescent signal, and hence the presence of A₂, is detectable.

In some embodiments, the double strand specific DNA digestion enzyme is an exonuclease.

In some embodiments, the double-strand specific DNA digestion enzyme is a polymerase with proofreading activity.

In some embodiments, the fluorophore is selected from dyes of the fluorescein family, the carboxyrhodamine family, the cyanine family, the rhodamine family, polyhalofluorescein-family dyes, hexachlorofluorescein-family dyes, coumarin-family dyes, oxazine-family dyes, thiazine-family dyes, squaraine-family dyes, and chelated lanthanide-family dyes.

In some embodiments, the fluorophore of the device may be selected from any of the commercially available dyes.

In some embodiments, the quencher of the device is selected from those available under the trade designations Black Hole™, Eclipse™ Dark, Qx1J, Iowa Black™, ZEN and/or TAO.

In some embodiments, the quencher of the device may be selected from any of the commercially available quencher.

In some embodiments, one or more wells of the second region may further comprise one or more partially double stranded DNA constructs wherein each construct contains one or more fluorophores and one or more quenchers. In some embodiments, when the construct is partially double-stranded the one or more fluorophores and one or more quenchers are located in close enough proximity to each other such that sufficient quenching of the one or more fluorophores occurs.

In some embodiments, the construct is one strand of DNA with a self-complementary region that is looped back on itself.

In some embodiments, the construct comprises one primer of a primer pair.

In some embodiments, one or more wells of the second region may further comprise the other primer of a primer pair.

In some embodiments, a portion of the single stranded section of the construct hybridises to A₂ and is extended against it by a DNA polymerase. In some embodiments, the other primer of the primer pair then hybridises to the extended construct, displaying A₂. This primer is then extended against the construct, displacing the self-complementary region. Thus, the one or more fluorophores and one or more dyes are separated sufficiently for a fluorescent signal to be detected, indicating the presence of A₂.

In such an embodiment the construct may be known as a Sunrise Primer.

In some embodiments, the construct comprises two separate DNA strands.

In some embodiments, a portion of the single stranded section of the construct hybridises to A₂ and is extended against it by a DNA polymerase. In some embodiments, the other primer of the primer pair then hybridises to the extended construct, displaying A₂. This primer is then extended against the construct, in the direction of the double stranded section, displacing the shorter of the DNA strands and thus the one or more fluorophores and one or more dyes are separated sufficiently for a fluorescent signal to be detected, indicating the presence of A₂.

In such an embodiment the construct may be known as a Molecular Zipper.

The person skilled in the art will appreciate that for both the Sunrise Primer and Molecular Zipper it is possible for the one or more fluorophores and the one or more quencher pairs to be located at various positions within each respective construct. The key feature is that each pair is located in sufficient proximity to one another that in the absence of A₂, i.e. when no extension and strand displacement has occurred, no fluorescent signal is emitted.

In some embodiments, one or more wells of one or more regions may further comprise a pyrophosphatase.

In some embodiments, one or more wells of one or more regions of the device may further comprise a phosphatase or a phosphohydrolase.

In some embodiments, one or more wells of the first region of the device may further comprise an enzyme for the formation of DNA from an RNA template.

In some embodiments, the enzyme is a reverse transcriptase.

In some embodiments, one or more enzymes present in the device are hot start.

In some embodiments, one or more enzymes present in the device are thermostable.

In some embodiments, the first and second regions of the device are combined.

In some embodiments of the invention there is provided a device comprising:
at least a fluid pathway between a first region, a second region and a third region, wherein the first region comprises one or more wells, wherein each well comprises:
dNTPs;
at least one single-stranded primer oligonucleotide;
an amplification enzyme for the initial amplification of DNA present in a sample; and

wherein the second region comprises one or more wells, wherein each well comprises:
a molecular system comprising a probe oligonucleotide A₀, capable of forming a first intermediate product with a target polynucleotide sequence, said intermediate product being at least partially double-stranded;
a pyrophosphorolysing enzyme capable of digesting the first intermediate product in the 3′-5′ direction from the end of A₀ to create a partially digested strand A₁; and

wherein the third region comprises one or more wells, wherein each well comprises:
dNTPs;
buffers;
optionally an amplification enzyme;
optionally a means for detecting a signal derived from A₂ or a portion thereof, or multiple copies of A₂ or multiple copies of a portion thereof; and

wherein the wells of the second region or the wells of the third region further comprise at least one single-stranded primer oligonucleotide that is substantially complementary to a portion of A₀.

In some embodiments, the wells of the second region comprise:
dNTPs;
one or more single-stranded primer oligonucleotides;
an amplification enzyme for the initial amplification of DNA present in a sample;

wherein one or more of the primers has a non-complimentary 5′ tail.

In some embodiments, one or more of the primers has a 5′ phosphate.

In some embodiments, one or more of the primers is 5′ protected.

In some embodiments, the pyrophosphorolysis enzyme which was present in the wells of the second region is carried through to the wells of third region wherein it performs amplification of A₂ in the presence of dNTPs and suitable buffers.

In some embodiments, a means for detecting a signal is located within one or more wells of the third region.

In some embodiments, a means for detecting a signal is located within the third region of the device.

In some embodiments, a means for detecting a signal is located within an adjacent region of the device.

In some embodiments, the dNTPs of each well of the first region may be dUTP, dGTP, dATP and dCTP and each well may further comprise a dUTP incorporating high fidelity polymerase and uracil-DNA N-glycosylase (UDG).

In some embodiments, each well of the second region may further comprise a source of pyrophosphate ion.

In some embodiments, the 5′ end of A₀ may be rendered resistant to 5′-3′ exonuclease digestion and the wells of the second region may further comprises a 5′-3′ exonuclease.

In some embodiments, each well of the second or third regions may further comprise a ligase.

In some embodiments, the dNTPs may be hot start.

In some embodiments, each well of the second region may further comprise a phosphatase or a phosphohydrolase.

In some embodiments, each well of the second region may further comprise a pyrophosphatase.

In some embodiments, the pyrophosphatase is hot start.

In some embodiments, each well of the third region may further comprise one or more oligonucleotide binding dyes or molecular probes.

In some embodiments, each well of the second region may comprise at least one or more different A₀ that is selective for a target sequence including an identification region.

In some embodiments, the amplification enzyme and the pyrophosphorolysing enzyme in the second region may be the same.

In some embodiments, there may be a fourth region comprising one or more wells, wherein each well may comprise a proteinase and wherein said fourth region may be located between the first and second regions.

In some embodiments, the second and third regions of the device may be combined such that the wells of the second region further comprise:
dNTPs;
buffers;
an amplification enzyme; and

a means for detecting a signal derived from A₁ or a portion thereof, or multiple copies of A₁ or multiple copies of a portion thereof.

In some embodiments, the second and third regions of the device may be combined such that the wells of the second region further comprise:
optionally dNTPs;
optionally an amplification enzyme;
buffers; and
labeled oligonucleotide probes.

In some embodiments, the pyrophosphorolysis enzyme which was present in the wells of the second region is utilised to perform amplification of A₂ in the presence of dNTPs and suitable buffers.

In some embodiments, a means for detecting a signal is located within one or more wells of the second region.

In some embodiments, a means for detecting a signal is located within the second region of the device.

In some embodiments, a means for detecting a signal is located within an adjacent region of the device.

In some embodiments, the first region may be fluidically connected to a sample container via a fluidic interface.

In some embodiments of the invention there is provided a device comprising:
at least a fluid pathway between a first second, third and fourth region, wherein the first region comprises one or more wells, wherein each well comprises means for selectively modifying a nucleic acid

wherein the second region comprises one or more wells, wherein each well comprises:
dNTPs;
at least one single-stranded primer oligonucleotide;
an amplification enzyme for the initial amplification of DNA present in a sample; and

wherein the third region comprises one or more wells, wherein each well comprises:
a molecular system comprising a probe oligonucleotide A₀, capable of forming a first intermediate product with a target polynucleotide sequence, said intermediate product being at least partially double-stranded;
a pyrophosphorolysing enzyme capable of digesting the first intermediate product in the 3′-5′ direction from the end of A₀ to create a partially digested strand A₁; and

wherein the fourth region comprises one or more wells, wherein each well comprises:
dNTPs;
buffers;
optionally an amplification enzyme;
a means for detecting a signal derived from A₂ or a portion thereof, or multiple copies of A₂ or multiple copies of a portion thereof; and

wherein the wells of the third region or the wells of the fourth region further comprise at least one single-stranded primer oligonucleotide that is substantially complementary to a portion of A₀. In some embodiments, the means for selectively modifying a nucleic acid may be chemicals capable of converting unmodified cytosine bases in a target polynucleotide sequence.

In some embodiments, the means for selectively modifying a nucleic acid may be enzymes capable of converting unmodified cytosine bases in a target polynucleotide sequence.

In some embodiments, the wells of the second or third region may further comprise a restriction endonuclease.

In some embodiments, located between the first and second region may be a region comprising one or more wells wherein each well may comprise a restriction endonuclease.

In some embodiments, the restriction endonuclease may recognise a sequence in a target polynucleotide sequence created by chemical or enzymatic conversion of unmodified cytosine bases.

In some embodiments, the sequence in a target polynucleotide sequence which the restriction endonuclease may recognise is removed by chemical or enzymatic conversion of unmodified cytosine bases.

In some embodiments, the restriction endonuclease may be a methylation-sensitive or methylation-dependent restriction endonuclease.

In some embodiments, the wells of the second region may comprise reagents for modification-specific multiplex ligation-dependent probe amplification (MS-MLPA) of epigenetically modified DNA.

In some embodiments, located between the first and second region may be a region comprising one or more wells wherein each well may comprise reagents for PCR.

In some embodiments, located between the first and second region may be a region comprising one or more wells wherein each well may comprise reagents for reduction of a population of epigenetically modified or unmodified target sequences.

In some embodiments, the reagents for reduction of a population of epigenetically modified or unmodified target sequences are reagents for epigenetically modified DNA immunoprecipitation, optionally methylated DNA immunoprecipitation (MeDIP).

In some embodiments, the reagents for reduction of a population of epigenetically modified or unmodified target sequences are methyl-binding proteins, such as MBD2b or the MBD2b/MBD3L1 complex.

In some embodiments, the reagents for reduction of a population of epigenetically modified or unmodified target sequences are located within one or more wells of the first region.

In some embodiments of the device, the epigenetic modification may be methylation. In some embodiments, it may be methylation at CpG islands. In some embodiments, it may be hydroxymethylation at CpG islands.

In some embodiments, the wells of the second, third or fourth region may comprise:
dNTPs;
at least one single-stranded primer oligonucleotide; and
an amplification enzyme.

In some embodiments, the dNTPs of each well may be dUTP, dGTP, dATP and dCTP and each well may further comprise a dUTP incorporating high fidelity polymerase and uracil-DNA N-glycosylase (UDG).

In some embodiments, each well may further comprise a source of pyrophosphate ion.

In some embodiments, the 5′ end of A₀ may be rendered resistant to 5′-3′ exonuclease digestion and the wells of the second or third region may further comprise a 5′-3′ exonuclease.

In some embodiments, each well of the third or fourth regions may further comprise a ligase.

In some embodiments, the dNTPs may be hot start.

In some embodiments, each well of the third region may further comprise a phosphatase or a phosphohydrolase.

In some embodiments, each well of the third region may further comprise a pyrophosphatase.

In some embodiments, each well of the fourth region may further comprise a pyrophosphatase.

In some embodiments, the pyrophosphatase is hot start.

In some embodiments, each well of the fourth region may further comprise one or more oligonucleotide binding dyes or molecular probes.

In some embodiments, each well of the third region may comprise at least one or more different A₀ that is selective for a target sequence including an identification region.

In some embodiments, the amplification enzyme in the fourth region and the pyrophosphorolysing enzyme in the third region may be the same, thus in some embodiments the amplification enzyme in the fourth region is not needed.

In some embodiments, there may be a fifth region comprising one or more wells, wherein each well may comprise a proteinase and wherein said fifth region may be located between the first and second regions.

In some embodiments, the fifth region may be located between the second and third regions.

In some embodiments, the third and fourth regions of the device may be combined such that the wells of the third region further comprise:
dNTPs;
buffers;
an amplification enzyme; and

a means for detecting a signal derived from A₁ or a portion thereof, or multiple copies of A₁ or multiple copies of a portion thereof.

In some embodiments, the means for detecting a signal are located within the third region.

In some embodiments, the means for detecting a signal are located within an adjacent region.

In some embodiments, the wells of the third or fourth region may further comprise:
two or more Ligation Chain Reaction (LCR) probe oligonucleotides that are complementary to adjacent sequences on A₁ wherein when the probes are successfully annealed the 5′ phosphate of one LCR probe is directly adjacent to the 3′OH of the other LCR probe; and
one or more ligases.

In some embodiments, the amplification enzyme and the pyrophosphorolysis enzyme of the device are the same.

In some embodiments, the wells of the third region may further comprise:
A hairpin oligonucleotide 1 (HO1) comprising a fluorophore-quencher pair, wherein HO1 is complementary to A₂ and when annealed to A₂ the hairpin structure of HO1 opens and the fluorophore-quencher pair separate; and
A hairpin oligonucleotide 2 (HO2) comprising a fluorophore-quencher pair, wherein HO2 is complementary to the open HO1 and when annealed to HO1 the hairpin structure of HO2 opens and the fluorophore-quencher pair separate.

In some embodiments, the wells of the third region may further comprise a plurality of HO1 and HO2.

In some embodiments, the wells of the third region may further comprise:
an oligonucleotide A comprising a substrate arm, a partial catalytic core and a sensor arm;
an oligonucleotide B comprising a substrate arm, a partial catalytic core and a sensor arm; and
a substrate comprising a fluorophore quencher pair;

wherein the sensor arms of oligonucleotides A and B are complementary to flanking regions of A₂ such that in the presence of A₂, oligonucleotides A and B are combined to form a catalytically, multicomponent nucleic acid enzyme (MNAzyme).

In some embodiments, the wells of the third region may comprise a partially double-stranded nucleic acid construct wherein:
one strand comprises at least one RNA base, at least one fluorophore and wherein a region of this strand is complementary to a region of A₂ and wherein this strand may be referred to as the ‘substrate’ strand; and
the other stand comprises at least one quencher and wherein a region of this strand is complementary to a region of A₂ adjacent to that which the substrate strand is complementary to, such that in the presence of A₂ the partially stranded nucleic acid construct becomes substantially more double-stranded.

In some embodiments, the wells of the third region may further comprise an enzyme for the removal of the at least one RNA base.

In some embodiments, the enzyme is Uracil-DNA Glycosylase (UDG) and the RNA base is uracil.

In some embodiments, one or more wells of the third region may further comprise:
an oligonucleotide complementary to a region of A₂ including the site of ligation, comprising one or multiple fluorophores arranged such that their fluorescence is quenched either by their proximity to each other or to one or more fluorescence quenchers;
a double strand specific DNA digestion enzyme;

wherein, in the presence of A₂, the labelled oligonucleotide is digested such that the fluorophores are separated from each other or from their corresponding quenchers, and a fluorescent signal, and hence the presence of A₂, is detectable.

In some embodiments, the double strand specific DNA digestion enzyme is an exonuclease.

In some embodiments, the double-strand specific DNA digestion enzyme is a polymerase with proofreading activity.

In some embodiments, the fluorophore is selected from dyes of the fluorescein family, the carboxyrhodamine family, the cyanine family, the rhodamine family, polyhalofluorescein-family dyes, hexachlorofluorescein-family dyes, coumarin-family dyes, oxazine-family dyes, thiazine-family dyes, squaraine-family dyes, and chelated lanthanide-family dyes.

In some embodiments, the fluorophore of the device may be selected from any of the commercially available dyes.

In some embodiments, the quencher of the device is selected from those available under the trade designations Black Hole™, Eclipse™ Dark, Qx1J, Iowa Black™, ZEN and/or TAO.

In some embodiments, the quencher of the device may be selected from any of the commercially available quencher.

In some embodiments, the wells of the third region may comprise one or more partially double stranded DNA constructs wherein each construct contains one or more fluorophores and one or more quenchers. In some embodiments, when construct is partially double-stranded the one or more fluorophores and one or more quenchers are located in close enough proximity to each other such that sufficient quenching of the one or more fluorophores occurs.

In some embodiments, the construct is one strand of DNA with a self-complementary region that is looped back on itself.

In some embodiments, the construct comprises one primer of a primer pair.

In some embodiments, the wells of the third region may further comprise the other primer of a primer pair.

In some embodiments, a portion of the single stranded section of the construct hybridises to A₂ and is extended against it by a DNA polymerase. In some embodiments, the other primer of the primer pair then hybridises to the extended construct. This primer is then extended against the construct, displacing the self-complementary region. Thus, the one or more fluorophores and one or more dyes are separated sufficiently for a fluorescent signal to be detected, indicating the presence of A₂ in the reaction mixture.

In such an embodiment the construct may be known as a Sunrise Primer.

In some embodiments, the construct comprises two separate DNA strands.

In some embodiments, a portion of the single stranded section of the construct hybridises to A₂ and is extended against it by a DNA polymerase. In some embodiments, the other primer of the primer pair then hybridises to the extended construct, displaying A₂. This primer is then extended against the construct, in the direction of the double stranded section, displacing the shorter of the DNA strands and thus the one or more fluorophores and one or more dyes are separated sufficiently for a fluorescent signal to be detected, indicating the presence of A₂ in the reaction mixture.

In such an embodiment the construct may be known as a Molecular Zipper.

The person skilled in the art will appreciate that for both the Sunrise Primer and Molecular Zipper it is possible for the one or more fluorophores and the one or more quencher pairs to be located at various positions within each respective construct. The key feature is that each pair is located in sufficient proximity to one another that in the absence of A₂, i.e. when no extension and strand displacement has occurred, no fluorescent signal is emitted.

In some embodiments, one or more wells of one or more regions may further comprise a pyrophosphatase.

In some embodiments, one or more wells of one or more regions of the device may further comprise a phosphatase or a phosphohydrolase.

In some embodiments, one or more wells of the second region of the device may further comprise an enzyme for the transcription of RNA into DNA.

In some embodiments, the enzyme is a reverse transcriptase.

In some embodiments, one or more enzymes present in the device are hot start.

In some embodiments, one or more enzymes present in the device are thermostable.

In some embodiments, the second and third regions of the device are combined.

In some embodiments, the third and fourth regions of the device are combined.

In some embodiments, there is located, between one or more wells of a region/and or between one or more regions of the device, one or more fluidic pathways.

In some embodiments, the first region may be fluidically connected to a sample container via a fluidic interface.

In some embodiments, heating and/or cooling elements may be present at one or more regions of the device.

In some embodiments, heating and/or cooling may be applied to one or more regions of the device.

In some embodiments, each region of the device may independently comprise at least 100 or 200 wells.

In some embodiments, each region of the device may independently comprise between about 100 and 300, 400, 500, 600, 700, 800, 900, 1000, 1100, 1200, 1300, 1400, 1500 or more wells. The wells may be of any shape and their locations may be arranged in any format or pattern on a substrate.

In some embodiments, the well-substrate can be constructed from a metal (e.g. gold, platinum, or nickel alloy as non-limiting examples), ceramic, glass, or other PCR compatible polymer material, or a composite material. The well-substrate includes a plurality of wells.

In some embodiments, the wells may be formed in a well-substrate as blind-holes or through-holes. The wells may be created within a well-substrate, for example, by laser drilling (e.g. excimer or solid-state laser), ultrasonic embossing, hot embossing lithography, electroforming a nickel mold, injection molding, and injection compression molding.

In some embodiments, individual well volume may range from 0.1 to 1500 nl. In one embodiment, 0.5 to 50 nL. Each well may have a volume of about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, or 500 nL.

In some embodiments, well dimensions may have any shape, for example, circular, elliptical, square, rectangular, ovoid, hexagonal, octagonal, conical, and other shapes well known to persons of skill in the art.

In some embodiments, well shapes may have cross-sectional areas that vary along an axis. For example, a square hole may taper from a first size to a second size that is a fraction of the first size.

In some embodiments, well dimensions may be square with diameters and depths being approximately equal.

In some embodiments, walls that define the wells may be non-parallel.

In some embodiments, walls that define the wells may converge to a point. Well dimensions can be derived from the total volume capacity of the well-substrate.

In some embodiments, well depths may range from 25 μm to 1000 μm. In one embodiment, wells may have a depth of 25, 50, 75, 100, 150, 200, 250, 300, 350, 400, 450, 500, 550, 600, 650, 700, 750, 800, 850, 900, 950 or 1000 μm.

In some embodiments, well diameter may range from about 25 μm to about 500 μm. In some embodiments, wells may have a width of 25, 50, 75, 100, 125, 150, 175, 200, 225, 250, 275, 300, 325, 350, 375, 400, 425, 450, 475 or 500 μm.

In some embodiments, portions of one or more regions of the device may be modified to encourage or discourage fluid adhered. Surfaces defining the wells may be coated with a hydrophilic material (or modified to be hydrophilic), and thus encourage retention of fluid.

In some embodiments, portions of one or more regions of the device, may be coated with a hydrophobic material (or modified to be hydrophobic) and thus discourage retention of fluid thereon. The person skilled in the art will understand that other surface treatments may be performed such that fluid is preferably held within the wells, but not on upper surfaces so as to encourage draining of excess fluid.

In some embodiments, the wells of the well-substrate may be patterned to have a simple geometric pattern of aligned rows and columns, or patterns arranged diagonally or hexagonally. In one embodiment, the wells of the well-substrate may be patterned to have complex geometric patterns, such as chaotic patterns or isogeometric design patterns.

In some embodiments, the wells may be geometrically separated from one another and/or feature large depth to width ratios to help prevent cross-contamination of reagents.

In some embodiments, the device may comprise one or more axillary regions which are usable to provide process fluids, such as oil or other chemical solutions to one or more of the regions of the device. Such auxiliary regions may be fluidically connected to one or more of the regions of the device via one or more membranes, valves and/or pressure severable substrates (i.e. materials that break when subjected to a pre-determined amount of pressure from fluid within an auxiliary region or adjacent portion of the fluid pathway) such as metal foil or thin film.

In some embodiments, the fluid pathway of the device may include extensive torturous portions. A torturous path between the inlet passage of the fluid pathway and one or more of the regions of the device can be helpful for control and handling of fluid processes. A torturous path can help reduce formation of gas bubbles that can interfere with flowing oil through the fluid pathway.

In some embodiments, the device may further comprises a gas permeable membrane which enables gas to be evacuated from the wells of one or more regions of the device, while not allowing fluid to pass through. The gas permeable membrane may be adhered to the well-substrate of the device by a gas permeable adhesive. In one embodiment, the membrane may be constructed from polydimethylsiloxane (PDMS), and has a thickness ranging from 20-1000 μm. In some embodiments the membrane may have a thickness ranging from 100-200 μm.

In some embodiments, all or portions of the well-substrate may contain conductive metal portions (e.g., gold) to enable heat transfer from the metal to the wells. In one embodiment, the interior surfaces of wells may be coated with a metal to enable heat transfer.

In some embodiments, after appropriate reagents have filled the wells of one or more regions of the device, an isolation oil or thermally conductive liquid may be applied to the device to prevent cross-talk.

In some embodiments, the wells of one or more regions of the device may be shaped to taper from a large diameter to a smaller diameter, similar to a cone. Cone-shaped wells with sloped walls enables the use of a non-contact deposition method for reagents (e.g., ink jet). The conical shape also aids in drying and has been found to prevent bubbles and leaks when a gas permeable membrane is present.

In some embodiments, the wells of one or more regions of the device may be filled by advancing a sample fluid (e.g. via pressure) along the fluid pathway of the device. As the fluid passes over the wells of one or more regions of the device, each well becomes filled with fluid, which is primarily retained within the wells via surface tension. As previously described, portions of the well-substrate of the device may be coated with a hydrophilic/hydrophobic substance as desired to encourage complete and uniform filing of the wells as the sample fluid passes over.

In some embodiments, the wells of one or more regions of the device may be ‘capped’ with oil following filling. This can then aid in reducing evaporation when the well-substrate is subjected to heat cycling. In one embodiment, following oil capping, an aqueous solution can fill one or more regions of the device to improve thermal conductivity.

In some embodiments, the stationary aqueous solution may be pressurised within one or more regions of the device to halt the movement of fluid and any bubbles.

In some embodiments, oil such as mineral oil may be used for the isolation of the wells of one or more regions of the device and to provide thermal conductivity. However, any thermal conductive liquid, such as fluorinated liquids (e.g., 3M FC-40) can be used. References to oil in this disclosure should be understood to include such alternatives as the skilled person in the art will appreciate are applicable.

In some embodiments, the device may further comprise one or more sensor assemblies.

In some embodiments, the one or more sensor assemblies may comprise a charge coupled device (CCD)/complementary metal-oxide-semiconductor (CMOS) detector coupled to a fiber optic face plate (FOFP). A filter may be layered on top of the FOPF, and placed against or adjacent to the well-substrate. In one embodiment, the filter can be layered (bonded) directly on top of the CCD with the FOPF placed on top.

In some embodiments, a hydration fluid, such as distilled water, may be heated within the first region or one of the auxiliary regions such that one or more regions of the device has up to 100% humidity, or at least sufficient humidity to prevent over evaporation during thermal cycling.

In some embodiments, after filing of the device is complete, the well-substrate may be heated by an external device that is in thermal contact with the device to perform thermal cycling for PCR.

In some embodiments, non-contact methods of heating may be employed, such as RFID, Curie point, inductive or microwave heating. These and other non-contact methods of heating will be well known to the person skilled in the art. During thermal cycling, the device may be monitored for chemical reactions via the sensor arrangements previously described.

In some embodiments, reagents that are deposited in one or more of the wells of one or more of the regions of the device are deposited in a pre-determined arrangement.

In some embodiments there is provided a method comprising:
providing a sample fluid to a fluid pathway of a device wherein the device comprises at least a fluid pathway between a first region, a second region and a third region, wherein the first, second and third regions independently comprise one or more wells;
filling the second region with the amplified fluid from the first region such that one or more wells of the second region is coated with the amplified fluid;
evacuating the amplified fluid from the second region such that one or more wells remain wetted with at least some of the amplified fluid;
filling the third region with the fluid evacuated from the second region such that one or more wells of the third region is coated with this fluid; and
evacuating the fluid from the third chamber such that the one or more wells remains wetted with at least some of this fluid.

In some embodiments of the method, the fluid pathway may be valveless.

In some embodiments of the method, the evacuated second region may be filled with a hydrophobic substance.

In some embodiments of the method, the evacuated third region may be filled with a hydrophobic substance.

In some embodiments of the method, the hydrophobic substance may be supplied from an oil chamber that is in fluid communication with the second and third regions.

In some embodiments of the method, the sample fluid may be routed along the fluid pathway in a serpentine manner.

In some embodiments, the method may further comprise applying heating and cooling cycles to the one or more of the first, second or third regions.

According to the present invention, there is provided a method for the detection of a polynucleotide target sequence, comprising the steps of:
a. introducing a sample comprising one or more nucleic acid analytes to an enrichment reaction mixture comprising:
   i. a molecular system comprising a probe molecule (A₀) and a hybridised splint molecule (C), wherein:
A₀ has a 3′-end which is complementary to the target polynucleotide sequence, a loop region and a 5′-phosphate; and
C is hybridised to the 5′-end of A₀ and provides a single stranded 3′-overhang, wherein the single stranded 3′-overhang can hybridise to a region located 1 to 50 bases in the 5′ direction from the 3′-end of A₀.
   ii. A capture oligonucleotide B₀ comprising a region complementary to a region adjacent to the target nucleic acid sequence and a capture moiety;
   iii. A solid support.
b. Allowing the molecular system and B₀ to hybridise to the nucleic acid analyte;
c. introducing the enrichment reaction mixture to a first reaction mixture comprising a pyrophosphorolysing enzyme, wherein A₀ is pyrophosphorolysed in the 3′-5′ direction from the 3′ end to create at least a partially digested strand A₁, wherein C displaces the 3′-end of A₁ from the target;
d. introducing the partially digested strand A₁ to a reaction mixture comprising a ligase, wherein A₁ undergoes ligation to form A₂; and
e. detecting a signal derived from the products of the previous step, wherein the products are A₂ or a portion thereof, or multiple copies of A₂ or multiple copies of a portion thereof, and inferring therefrom the presence or absence of the polynucleotide target sequence in the analyte.

In some embodiments, the reaction mixture of (d) is combined with the first reaction mixture such that the ligase is present in the same reaction mixture as the pyrophosphorolysis enzyme.

The person skilled in the art will appreciate that previous or subsequent descriptions of the first and second reaction mixtures in the context of other embodiments may be equally applicable to the above embodiment and this application includes within its scope all such variations.

The final step of the method may be achieved as described previously or subsequently.

In some embodiments, the one or more nucleic acid analytes are split into multiple reaction volumes, each volume having one or more molecular systems comprising a probe oligonucleotide A₀, and one or more capture oligonucleotide B₀, introduced to detect different target sequences.

In some embodiments, B₀ is attached to the solid support, prior to step (b).

In some embodiments, B₀ is attached to the solid support between steps (b) and (c).

In some embodiments, following attachment of B₀ to the solid support and hybridisation of A₀ and B₀ to the target, the supernatant and thus any A₀ which are not hybridised to the target nucleic acid analyte are removed from the reaction mixture.

This removal can occur prior to (c).

This removal can occur prior to (d).

In some embodiments, the capture oligonucleotide is complementary to a region that is within 10000, 5000, 2500, 1000, 500, 250, 100, 90, 80, 70, 60, 50, 40, 30, 20, 10 or 5 nucleotides of the target nucleic acid sequence.

In some embodiments, the capture oligonucleotide is complementary to a region that is within 100 nucleotides of the target nucleic acid sequence.

In some embodiments, the capture oligonucleotide is complementary to a region that is within 50 nucleotides of the target nucleic acid sequence.

In some embodiments, the capture oligonucleotide is complementary to a region that is within 10 nucleotides of the target nucleic acid sequence.

In some embodiments, the oligonucleotides A₀ and B₀ are joined to form a single oligonucleotide.

In some embodiments, the solid support is a polymer and/or resin coated solid surface.

In some embodiments, the solid support is a polystyrene solid support.

In some embodiments, polystyrene (C₈H₈)ₙ is a polymer wherein n is 10, 20, 30, 40, 50, 100, 150, 200, 250, 300, 350, 400, 450, 500, 550, 600, 650, 700, 750, 800, 850, 900, 950, 1,000, 1,250, 1,500, 1,750, 2,000, 2,500, 3,000, 3,500, 4,000, 4,500, 5,000, 5,500, 6,000, 6,500, 7,000, 7,500, 8,000, 8,500, 9,000, 9,500, 10,000, 11,000, 12,000, 12,500, 15,000, 16,000, 17,000, 17,500, 18,000, 19,000, 20,000, 25,000, 30,000, 40,000, 50,000, 60,000, 70,000, 80,000, 90,000, 100,000 or more, or where n is any integer between any of these points, or n is within any range derivable between any two of these points, is utilised.

In some embodiments, the polystyrene solid support is a particle, micro particle, magnetic bead, resin or any particulate that comprises polystyrene polymers.

In some embodiments, the polystyrene support is modified to include one or more of the following functional groups: amine, carboxylate, sulfonate, trimethylamine and/or epoxide.

In some embodiments, the solid support is a magnetic bead.

In some embodiments, the magnetic bead is a shape which maximises the surface area of said bead.

In some embodiments, the magnetic bead is a regular shape.

In some embodiments, the magnetic bead is an irregular shape.

In some embodiments, the magnetic bead has a diameter of less than, or equal to: 1000, 900, 800, 700, 600, 500, 400, 300, 200, 100, 90, 80, 70, 60, 50, 40, 30, 20, 10, 5, 2.5, 1, 0.5, 0.25 or 0.1 micron.

In some embodiments, the solid support is a magnetic polystyrene bead.

In some embodiments, the magnetic polystyrene bead comprises iron oxide.

In some embodiments, the magnetic polystyrene bead is Streptavidin-coupled.

In some embodiments, the solid support is a Streptavidin-coupled Dynabead®.

In some embodiments, the solid support is a dextran-modified surface.

In some embodiments, the dextran-modified surface is a particle, micro particle, magnetic bead, resin or any particulate that comprises dextran polymers.

In some embodiments, the dextran polymers have an approximate molecular weight from 1000 to 410000.

In some embodiments, the dextran polymers have an approximate molecular weight from 25000 to about 100000.

In some embodiments, the dextran-surface modified is further modified to include one or more functional groups.

In some embodiments, the dextran-modified surface is modified to include one or more of the following functional groups: amine, carboxylate, sulfonate, trimethylamine and/or epoxide.

In some embodiments, the solid support is a magnetic bead.

In some embodiments, the magnetic bead is a shape which maximises the surface area of said bead.

In some embodiments, the magnetic bead is a regular shape.

In some embodiments, the magnetic bead is an irregular shape.

In some embodiments, the magnetic bead has a diameter of less than, or equal to: 1000, 900, 800, 700, 600, 500, 400, 300, 200, 100, 90, 80, 70, 60, 50, 40, 30, 20, 10, 5, 2.5, 1, 0.5, 0.25 or 0.1 micron.

In some embodiments, the magnetic bead is a dextran magnetic bead selected from: Nanomag® Dextran (ND); Nanomag® Dextran-SO3H (ND-SO3H); BioMag® Dextran-Coated Charcoal; or BioMag® Plus Dextran.

In some embodiments, the solid support is a Polyethylene Glycol (PEG) or PEG-modified surface.

In some embodiments, the Polyethylene Glycol (PEG) or PEG-modified surface is a particle, micro particle, magnetic bead, resin or any particulate that comprises PEG.

In some embodiments of the invention PEG, (CH₂O)ₙ, is a polymer wherein n is 10, 20, 30, 40, 50, 100, 150, 200, 250, 300, 350, 400, 450, 500, 550, 600, 650, 700, 750, 800, 850, 900, 950, 1,000, 1,250, 1,500, 1,750, 2,000, 2,500, 3,000, 3,500, 4,000, 4,500, 5,000, 5,500, 6,000, 6,500, 7,000, 7,500, 8,000, 8,500, 9,000, 9,500, 10,000, 11,000, 12,000, 12,500, 15,000, 16,000, 17,000, 17,500, 18,000, 19,000, 20,000, 25,000, 30,000, 40,000, 50,000, 60,000, 70,000, 80,000, 90,000, 100,000 or more, or where n is any integer between any of these points, or n is within any range derivable between any two of these points, is utilised.

In some embodiments, the PEG utilised is PEG-200, PEG-300, PEG-400, PEG-600, PEG-1000, PEG-1300-1600, PEG-1450, PEG-3000-3700, PEG-3500, PEG-6000, PEG-8000 or PEG-17500.

In some embodiments, wherein the Polyethylene Glycol (PEG) or PEG-modified surface is a magnetic microparticle or bead, the microparticle or bead is selected from Nanomag® PEG-300 (Plain) or Nanomag®-D.

In some embodiments, the solid support is a Polyvinylpyrrolidone (PVP) or PVP-modified surface.

In some embodiments, the PVP or PVP-modified surface is a particle, micro particle, magnetic bead, resin or any particulate that comprises PVP.

In some embodiments of the invention PVP, n-vinyl pyrrolidone, is a polymer wherein n is 10, 20, 30, 40, 50, 100, 150, 200, 250, 300, 350, 400, 450, 500, 550, 600, 650, 700, 750, 800, 850, 900, 950, 1,000, 1,250, 1,500, 1,750, 2,000, 2,500, 3,000, 3,500, 4,000, 4,500, 5,000, 5,500, 6,000, 6,500, 7,000, 7,500, 8,000, 8,500, 9,000, 9,500, 10,000, 11,000, 12,000, 12,500, 15,000, 16,000, 17,000, 17,500, 18,000, 19,000, 20,000, 25,000, 30,000, 40,000, 50,000, 60,000, 70,000, 80,000, 90,000, 100,000 or more, or where n is any integer between any of these points, or n is within any range derivable between any two of these points, is utilised.

In some embodiments, the solid support is a polysaccharide or polysaccharide-modified surface. In some embodiments, the polysaccharide or polysaccharide-modified surface is a particle, micro particle, magnetic bead, resin or any particulate that comprises a polysaccharide.

In some embodiments, the polysaccharide is selected from one or more of dextran, ficoll, glycogen, gum arabic, xanthan gum, carageenan, amylose, agar, amylopectin, xylans and/or beta-glucans.

In some embodiments, the solid support is a chemical resin or chemical resin-modified surface.

In some embodiments, the chemical resin or chemical-resin modified surface is selected from one or more of the following resins: isocyanate, glycerol, piperidino-methyl, polyDMAP (polymer-bound dimethyl 4-aminopyridine), DIPAM (Diisopropylaminomethyl, aminomethyl, polystyrene aldehyde, tris(2-aminomethyl) amine, morpholino-methyl, BOBA (3-Benzyloxybenzaldehyde), triphenyl-phosphine or benzylthio-methyl.

In some embodiments, B₀ is covalently attached to the solid support via the capture moiety of B₀.

In some embodiments, the capture moiety of B₀ is covalently attached to the solid support via a chemically-cleavable linker, such as a disulfide, allyl, or azide-masked hemiaminal ether linker.

In some embodiments, the capture moiety of B₀ is covalently attached to the solid support via amide or phosphorothioate bonds.

The person skilled in the art will appreciate that there exists a plethora of techniques for the covalent and non-covalent immobilisation of oligonucleotides to solid supports, see for example “Strategies for Attaching Oligonucleotides to Solid Supports.” (2014) produced by Integrated DNA Technologies (IDT®).

In some embodiments, B₀ is non-covalently attached to the solid support via the capture moiety of B₀.

The person skilled in the art will appreciate that there exists a plethora of techniques for the covalent and non-covalent immobilisation of oligonucleotides to solid supports, see for example “Strategies for Attaching Oligonucleotides to Solid Supports.” (2014) produced by Integrated DNA Technologies (IDT®).

In some embodiments, the capture moiety of B₀ comprises an oligonucleotide sequence and the solid support comprises oligonucleotides bearing the complementary sequence.

In some embodiments, the length of the complementary sequence is between 10, 20, 30, 40, 50, 100, 150 and 200 bases.

In some embodiments, the length of the complementary sequence is between 10, 20, 30, 40, 50, and 100 bases.

In some embodiments, the length of the complementary sequence is between 10-20, 10-30, 10-40 and 10-50 bases.

In some embodiments, the length of the complementary sequence is between 10-20, 10-30 and 10-40 bases.

In some embodiments, the length of the complementary sequence is between 10-20 and 10-30 bases.

In some embodiments, the length of the complementary sequence is between 10-20 bases.

In some embodiments, the capture moiety comprises a chemical modification to B₀ and B₀ is attached to the solid support via an interaction between the chemical modification and the solid support.

In some embodiments, the chemical modification is biotin and the solid support further comprises streptavidin.

In some embodiments of the method, the first reaction mixture comprises multiple different oligonucleotides A₀ and B₀ and wherein the successfully hybridised A₀ oligonucleotides are simultaneously enriched.

In some embodiments, prior to step (c) A₀, the target nucleic acid, and optionally B₀ are released from the solid support.

In some embodiments, following step (c) A₁, optionally B₀ and optionally the target nucleic acid are released from the solid support.

In some embodiments, during step (c) A₁, optionally B₀ and optionally the target nucleic acid are released from the solid support.

It should be understood that references to release of A₀ encompass embodiments wherein A₀ is converted to A₁ or A₂ whilst solid surface bound and then released.

In some embodiments, A₀, B₀ and the target nucleic acid are released from the solid support by the cleavage of a chemical linker through the addition of tris(2-carboxyethyl)phosphine (TCEP) or dithiothreitol (DTT) for a disulfide linker; palladium complexes or an allyl linker; or TCEP for an azide-masked hemiaminal ether linker.

In some embodiments, A₀, B₀ and the target nucleic acid are released from the solid support by removing a non-canonical base, from A₀ or B₀, and cleavage at the resultant abasic site. In some embodiments, the non-canonical base is uracil, which is removed by uracil DNA glycosylase. In an alternate embodiment, the non-canonical base is 8-oxoguanine, which is removed by formamidopyrimidine DNA glycosylase (Fpg).

In some embodiments, the capture moiety is an oligonucleotide region and release is performed through heating of the reaction mixture.

In some embodiments, the reaction mixture is heated to 37° C.-100° C. over 1-20 minutes.

In some embodiments, the reaction mixture is heated over 1-15 minutes.

In some embodiments, the reaction mixture is heated over 1-10 minutes.

In some embodiments, the reaction mixture is heated over 1-5 minutes.

In some embodiments, the reaction mixture is heated over 5 minutes.

In some embodiments, the reaction mixture is heated to 37° C.-85° C.

In some embodiments, the reaction mixture is heated to 37° C.-75° C.

In some embodiments, the reaction mixture is heated to 37° C.-65° C.

In some embodiments, the reaction mixture is heated to 37° C.-55° C.

In some embodiments, the reaction mixture is heated to 37° C.-45° C.

The person skilled in the art will appreciate that the temperature to which the reaction mixture is heated so enact release of complementary oligonucleotide regions depends on the length of said regions.

In some embodiments, release is achieved through the cleavage of oligonucleotide B₀. This cleavage can be achieved by any of the means previously, or subsequently, described or by any of the means known to the person skilled in the art.

In some embodiments, B₀ is cleaved chemically.

In some embodiments, B₀ is cleaved enzymatically.

In some embodiments, B₀ is cleaved by a restriction enzyme.

In some embodiments, B₀ is cleaved by epigenetic modification sensitive or dependent restriction enzymes.

In some embodiments, B₀ is cleaved by methylation sensitive or dependent restriction enzymes.

In some embodiments, B₀ is cleaved by hydroxymethylation sensitive or dependent restriction enzymes.

In some embodiments, the restriction enzymes are endonucleases.

In some embodiments, B₀ is cleaved by a flap endonuclease.

In some embodiments, B₀ comprises a photocleavable linker and A₀, B₀ and the target nucleic acid are released from the solid support by cleavage of this linker.

In some embodiments, B₀ comprises a UV cleavable linker and A₀, B₀ and the target nucleic acid are released from the solid support by cleavage of this linker.

In some embodiments, release is achieved through the cleavage of A₀ with a methylation-sensitive or methylation-dependent restriction enzyme. In some embodiments, release is achieved through the cleavage of A₀ and the nucleic acid analyte.

In some embodiments, wherein A₀ and B₀ are regions of the same oligonucleotide Co, Co is cleaved with a methylation-sensitive or methylation-dependent restriction enzyme and the portion comprising A₀ and the nucleic acid analyte are released from the solid support.

In some embodiments, release occurs prior to pyrophosphorolysis.

In some embodiments, release occurs simultaneously with pyrophosphorolysis. In some embodiments, target nucleic acid sequence hybridised A₀ is prevented from undergoing pyrophosphorolysis, prior to release from the solid support, by the presence of modifications or mismatches at its 3′ end. In some embodiments, cleavage occurs in the 5′ direction from these modifications or mismatches, thus creating a free 3′ end which allows the pyrophosphorolysis reaction to proceed.

In some embodiments, cleavage and release only occurs in the presence of methylation in the target nucleic acid sequence.

In some embodiments, cleavage and release only occurs in the absence of methylation in the target nucleic acid sequence.

In some embodiments, cleavage and release only occurs in the presence of hydroxymethylation in the target nucleic acid sequence.

In some embodiments, cleavage and release only occurs in the absence of hydroxymethylation in the target nucleic acid sequence.

In some embodiments, cleavage and release only occurs in the presence of an epigenetic modification in the target nucleic acid sequence.

In some embodiments, cleavage and release only occurs in the absence of an epigenetic modification in the target nucleic acid sequence.

In some embodiments of the invention there is provided a kit comprising:
(a) a molecular system comprising a probe oligonucleotide A₀, capable of forming a first intermediate product with a target polynucleotide sequence, said intermediate product being at least partially double-stranded;
(b) A capture oligonucleotide B₀ comprising a region complementary to a region adjacent to the target nucleic acid sequence and a capture moiety;
(c) A solid support;
(d) a ligase;
(e) a pyrophosphorolyising enzyme capable of digesting the first intermediate product in the 3′-5′ direction from the end of A₀ to create a partially digested strand A₁; and
(f) suitable buffers.

The person skilled in the art will appreciate that the kit may further comprise one or more of any reagent, component or construct which has been previously described in embodiments of one or more of the methods of the invention.

In some embodiments of the invention there is provided a device comprising at least a fluid pathway between a first, second, third, fourth, fifth and sixth regions, wherein each region comprises one or more wells.

In some embodiments, a sample is introduced to the first region.

In some embodiments, there is a further region connected to one or more of the first, second, third, fourth, fifth or sixth regions by a fluid pathway. In some embodiments, this further region comprises one or more wells. In some embodiments, the one or more wells comprise binding buffer. In some embodiments, the one or more wells comprises wash buffer. In some embodiments, the one or more wells comprise binding and wash buffer.

In some embodiments, the one or more wells of the first region comprise:
A molecular system comprising a probe oligonucleotide A₀ including a region complementary to a target nucleic acid sequence;
A capture oligonucleotide B₀ comprising a region complementary to a region adjacent to the target nucleic acid sequence and a capture moiety; and
A solid support.
In some embodiments, A₀ may be as previously described.

In some embodiments, B₀ may be as previously described.

In some embodiments, the capture moiety is as previously described. In some embodiments, the one or more wells of the first region comprise the reagents necessary for release of B₀ from the solid support. In some embodiment, the device is arranged such that release agents are introduced to one or more regions from one or more separate chambers.

In some embodiments, the solid support is as previously described.

In some embodiments, the solid support is the surface of the one or more wells.

In some embodiments, the device further comprises one or more magnets. In some embodiments, the one or more magnets are arranged such that the one or more magnetic beads are capable of being moved from one region of the device to another.

The person skilled in the art will appreciate that the one or more regions of one or more wells of the device may be as described previously or subsequently. Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments, it is not limited to the disclosed embodiments and that alternative embodiments could be constructed without departing from the scope of the invention as defined in the appended claims.

As used herein, “magnetic microparticles” are magnetically responsive microparticles which are attracted by a magnetic field. The magnetic microparticles used in the methods of the present invention comprise a magnetic metal oxide core, which is generally surrounded by a polymer coat which creates a surface that can bind to DNA, RNA, or PNA. The magnetic metal oxide core is preferably iron oxide, wherein iron is a mixture of Fe²⁺ and Fe³⁺. The preferred Fe²⁺/Fe³⁺ ratio is preferably 2/1, but can vary from about 0.5/1 to about 4/1.

The person skilled in the art will appreciate that when the term ‘infer’ is used, for example, ‘infer the presence of absence of a particular sequence’ it refers to determining the presence or absence of a particular feature based on presence of absence of A₂, or copies of A₂ or a region of A₂ or copies of a region A₂.

The person skilled in the art will appreciate that embodiments wherein ‘primers’ are described include within their scope primers which are as described previously or subsequently in this document.

The person skilled in the art will appreciate that embodiments wherein ‘primers’ are described as being located within particular regions/wells of a device, or present in particular reaction mixtures, include within their scope embodiments wherein one or more blocking oligonucleotides, as previously or subsequently described, are also present within the same regions/wells or reaction mixtures.

The person skilled in the art will appreciate that embodiments wherein ‘a molecular system comprising a probe oligonucleotide A₀’ is described as being located within particular regions/wells of a device, or present in particular reaction mixtures, include within their scope embodiments wherein one or more blocking oligonucleotides, as previously or subsequently described, are also present within

### Example 1: Effect of Length of Annealing Region Between Probe and Target on the Optimum Temperature for the Pyrophosphorolysis Reaction

1. Polymerase Chain Reaction

A mixture was prepared corresponding to:
1×Q5U buffer
400 nM primer mix 1
20 U/mL Q5U Polymerase
10 U/mL Thermolabile UDG
0.4 ng/uL fragmented human genomic DNA
+/−0.2 aM mutant oligonucleotide
Total volume 50 uL

This mixture was then incubated as follows, using a standard thermal cycler:

| 37° C. | 10 | mins |
|---|---|---|
| 98° C. | 1 | min |
| 98° C. | 10 | sec |
| 58° C. | 15 | sec |
| 72° C. | 15 sec × 50 | |
| 72° C. | 5 | mins |

| | |
|---|---|
| 4° C. | ∞ |

Q5 Buffer

The Q5 buffer is available from commercial supplier NEB.

Primer Mix 1:

Fwd (SEQ ID NO 36):
5′-C*C*C*AACCAAGCTCTCTTGAGGATCT-3′
Rev (SEQ ID NO 37):
5′-/5Phos/GGGACCTTACCTTATACACCGTGC-3′

where * represents a phosphorothioate bond

Mutant oligonucleotide SEQ ID NO 38):

5′-CCCAACCAAGCTCTCTTGAGGATCTTGAAGGAAACTGAATTC
AAAAAGATCAAAGTGCTGGCCTCCGGTGCGTTCGGCACGGTGTAT
AAGGTAAGGTCCC-3′

Bold and underlined—mutation site

2. Proteinase K Treatment

A mixture was prepared corresponding to:
1×A7
20 U/mL Proteinase K
40 uL of mixture point 1
Total volume 90 uL

This mixture was then incubated at 55° C. for 5 min, 95° C. for 10 min.

1×A7 Composition
Tris Acetate pH=8.0 10 mM
Potassium Acetate 25 mM
Magnesium Acetate 5 mM
Triton-X 0.01%

3. Pyrophosphorolysis (PPL) and Ligation

A mixture was prepared corresponding to:
1×BFF1
10 U/mL Klenow (exo-)
100 U/mL E. coli Ligase
1.2 U/mL apyrase
100 U/mL Lambda exo
0.25 mM PPi
Molecular system (20 nM probe oligonucleotide/30 nM splint oligonucleotide) 1.25 uL of mixture from point 2.
Total volume 10 uL

This mixture was then incubated at 30-50° C. for 15 min.

1×BFF1 Composition
Tris Acetate pH=7.0 10 mM
Potassium Acetate 30 mM
Magnesium Acetate 17.125 mM
Triton-X 0.01%

Probe (SEQ ID NO 1):
5′-/5Phos/A*T*G*TTCGATGAGCTTTGACAATACTTGACAT
GCGCAGATATAGGATGTTGCCGAACGCACCGGAGGCCAGCACTT
TG-3′
Splint oligonucleotide (SEQ ID NO 2):
5′-TGTCAAAGCTCATCGAACATCCGGTGCGTTCGGCAT-3′
Probe (SEQ ID NO 3):
5′-/5Phos/A*T*G*TTCGATGAGCTTTGACAATACTTGACAT
GCGCAGATATAGGATGTTGCGGAACGCACCGGAGGCCAGCACTT
TG-3′
Splint oligonucleotide (SEQ ID NO 4):
5′-TGTCAAAGCTCATCGAACATCCGGTGCGTTCGGCAT-3′
Probe (SEQ ID NO 5):
5′-/5Phos/A*T*G*TTCGATGAGCTTTGACAATACTTGACAT
GCGCAGATATAGGATGTTGCGAAACGCACCGGAGGCCAGCACTT
TG-3′
Splint oligonucleotide (SEQ ID NO 6):
5′-TGTCAAAGCTCATCGAACATCCGGTGCGTTCGGCAA-3′
Probe (SEQ ID NO 7):
5′-/5Phos/A*T*G*TTCGATGAGCTTTGACAATACTTGACAT
GCGCAGATATAGGATGTTGCGATACGCACCGGAGGCCAGCACTT
TG-3′
Splint oligonucleotide (SEQ ID NO 8):
5′-TGTCAAAGCTCATCGAACATCCGGTGCGTATCGCAT-3′
Probe (SEQ ID NO 9):
5′-/5Phos/A*T*G*TTCGATGAGCTTTGACAATACTTGACAT
GCGCAGATATAGGATGTTGCGATTCGCACCGGAGGCCAGCACTT
TG-3′
Splint oligonucleotide (SEQ ID NO 10):
5′-TGTCAAAGCTCATCGAACATCCGGTGCGAATCGCAT-3′
Probe (SEQ ID NO 11):
5′-/5Phos/A*T*G*TTCGATGAGCTTTGACAATACTTGACAT
GCGCAGATATAGGATGTTGCGATTGGCACCGGAGGCCAGCACTT
TG-3′
Splint oligonucleotide (SEQ ID NO 12):
TGTCAAAGCTCATCGAACATCCGGTGCCAATCGCAT
Probe (SEQ ID NO 13):
5′-/5Phos/A*T*G*TTCGATGAGCTTTGACAATACTTGACAT
GCGCAGATATAGGATGTTGCGATTGCCACCGGAGGCCAGCACTT
TG-3′
Splint oligonucleotide (SEQ ID NO 14):
5′-TGTCAAAGCTCATCGAACATCCGGTGGCAATCGCAT-3′
Probe (SEQ ID NO 15):
5′-/5Phos/A*T*G*TTCGATGAGCTTTGACAATACTTGACAT
GCGCAGATATAGGATGTTGCGATTGCGACCGGAGGCCAGCACTT
TG
Splint oligonucleotide (SEQ ID NO 16):
5′-TGTCAAAGCTCATCGAACATCCGGTCGCAATCGCAT-3′

where * represents a phosphorothioate bond and /5Phos/ represents 5′ end phosphate

4. Detection—RCA

A mixture corresponding to the following was prepared:
2.66× Thermopol buffer (53.2 mM Tris-HCl, 26.6 mM (NH₄)₂SO₄, 26.6 mM KCl, 5.32 mM MgSO₄, 0.266% Triton® X-100, pH 8.8)
0.28 uM Primer mix 1
284.4 U/mL BST 2.0 WarmStart
14.67 U/mL TIPP
1.06 mM dNTPs
Syto82 dye 3 uM
1.25 uL of reaction mixture from point 3.
Total volume 11.25 uL

Primer mix 1:
Fwd (SEQ ID NO 17):
5′-T*C*GCAACATCCTATATCTGC-3′
Rev (SEQ ID NO 18):
5′-T*G*AGCTTTGACAATACTTGA-3′

where * represents a phosphorothioate bond

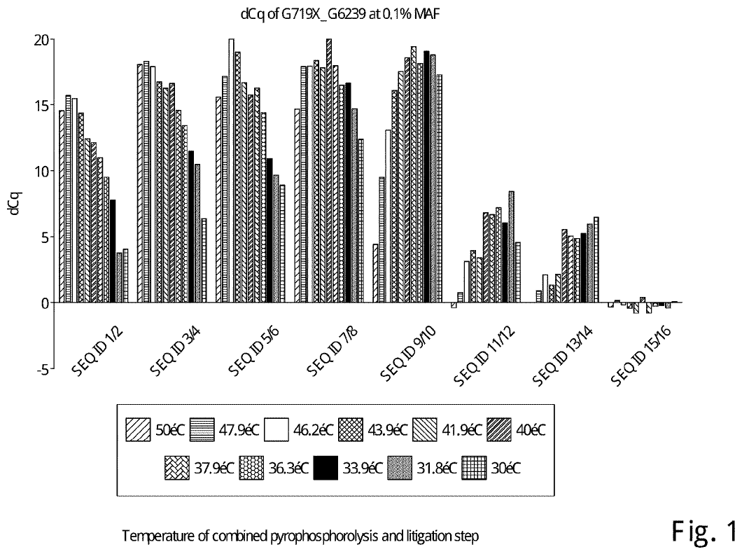

The mixture was then incubated at 50° C. for 90 min using a standard real-time PCR instrument. Fluorescent measurements were taken every 1 minute and a Cq value obtained using the standard instrument manufacturer software. The difference in Cq between the wildtype and mutant-containing samples can be seen in FIG. 1.

The length of the annealing region is summarised in Table 1. The longer the length of the annealing region between the probe and the target molecule, the higher the optimum temperature. For a complementarity of 29 base pairs (SEQ ID 1/2) the optimum is between 46-50° C., for 22 base pairs 30-42° C.

Too short of an annealing region can lead to a decrease in dCq (SEQ ID 11/12 and 13/14). With a 19 base pair length, it was not possible to detect a positive signal indicating the presence of the target molecule (SEQ ID 15/16).

TABLE 1
The length of the complementary region
between the probe and target molecule.

| SEQ ID | Complementary between probe and target molecule [bp] |
|---|---|
| 1/2 | 29 |
| 3/4 | 25 |
| 5/6 | 24 |
| 7/8 | 23 |
| 9/10 | 22 |
| 11/12 | 21 |
| 13/14 | 20 |
| 15/16 | 19 |

### Example 2: Effect of the Length of Annealing Region Between Probe, its Target and its Splint

1. Polymerase Chain Reaction

A mixture was prepared corresponding to:
1×Q5U buffer
400 nM primer mix 1
20 U/mL Q5U Polymerase
10 U/mL Thermolabile UDG
0.4 ng/uL fragmented human genomic DNA
+/−0.2 aM mutant oligonucleotide
Total volume 50 uL

This mixture was then incubated as follows using a standard thermal cycler:

| 37° C. | 10 | mins |
|---|---|---|
| (98° C. | 1 | min |
| 98° C. | 10 | sec |
| 58° C. | 15 | sec |
| 72° C. | 15 sec) × 50 | |
| 72° C. | 5 | mins |

| | |
|---|---|
| 4° C. | ∞ |

Q5 Buffer

The Q5 buffer is available from commercial supplier NEB.

Primer mix 1:
Fwd (SEQ ID NO 19):
5′-A*C*G*TACTGGTGAAAACACCGC-3′
Rev (SEQ ID NO 20):
5′-/5Phos/GCCTCCTTCTGCATGGTATTCT-3′

where * represents a phosphorothioate bond

Mutant Oligonucleotide (SEQ ID NO 21):

5′-ACGTACTGGTGAAAACACCGCAGCATGTCAAGATCACAGATTTT
GGGCTGGCCAAACAGCTGGGTGCGGAAGAGAAAGAATACCATGCAGA
AGGAGGC-3′

Bold and underlined—mutation site

2. Proteinase K Treatment

A mixture was prepared corresponding to:
1×A7
20 U/mL Proteinase K
40 uL of mixture point 1
Total volume 90 uL

This mixture was then incubated at 55° C. for 5 min, 95° C. for 10 min.

1×A7 Composition
Tris Acetate pH=8.0 10 mM
Potassium Acetate 25 mM
Magnesium Acetate 5 mM
Triton-X 0.01%

3. Pyrophosphorolysis (PPL) and Ligation

A mixture was prepared corresponding to:
1×BFF1
10 U/mL Klenow (exo-)
100 U/mL E. coli Ligase
1.2 U/mL apyrase
100 U/mL Lambda exo
0.25 mM PPi
20 nM probe oligonucleotide
30 nM splint oligonucleotide
1.25 uL of mixture from point 2.
Total volume 10 uL

This mixture was then incubated at 45° C. for 15 min.

1×BFF1 Composition
Tris Acetate pH=7.0 10 mM
Potassium Acetate 30 mM
Magnesium Acetate 17.125 mM
Triton-X 0.01%

Probe oligonucleotide (SEQ ID NO 22):
5′-/5Phos/A*TGTTCGATGAGCTTTGACAATACTTGATCGATGCA
GATATAGGATGTTGCGACGCACCCAGCTGTTTGG-3′
Probe oligonucleotide (SEQ ID NO 23):
5′-/5Phos/A*T*G*TTCGATGAGCTTTGACAATACTTGATCGATG
CAGATATAGGATGTTGCGACGCACCCAGCTGTTTGGCCAGCCCAAAA
TCTGTGAT-3′
where * represents a phosphorothioate bond,
/5Phos/ represents phosphate at 5′ end
Splint oligonucleotide (SEQ ID NO 24):
5′-TGTCAAAGCTCATCGAACATTGGGTGTCGCAACATG-3′
Splint oligonucleotide (SEQ ID NO 25):
5′-TGTCAAAGCTCATCGAACATGTCGCAACAGAAGTCGCAACATG-3′
Splint oligonucleotide (SEQ ID NO 26):
5′-TGTCAAAGCTCATCGAACATCGTCGCAACGAAGTCGCAACATG-3′
Splint oligonucleotide (SEQ ID NO 27):
5′-TGTCAAAGCTCATCGAACATGCGTCGCAAGAAGTCGCAACATG-3′
Splint oligonucleotide (SEQ ID NO 28):
5′-TGTCAAAGCTCATCGAACATTGCGTCGCAGAAGTCGCAACATG-3′
Splint oligonucleotide (SEQ ID NO 29):
5′-TGTCAAAGCTCATCGAACATGTGCGTCGCGAAGTCGCAACATG-3′
Splint oligonucleotide (SEQ ID NO 30):
5′-TGTCAAAGCTCATCGAACATGGTGCGTCGGAAGTCGCAACATG-3′
Splint oligonucleotide (SEQ ID NO 31):
5′-TGTCAAAGCTCATCGAACATGGGTGCGTCGAAGTCGCAACATG-3′
Splint oligonucleotide (SEQ ID NO 32):
5′-TGTCAAAGCTCATCGAACATTGGGTGCGTGAAGTCGCAACATG-3′
Splint oligonucleotide (SEQ ID NO 33):
5′-TGTCAAAGCTCATCGAACATCTGGGTGCGGAAGTCGCAACATG-3′
Splint oligonucleotide (SEQ ID NO 34):
5′-TGTCAAAGCTCATCGAACATGCTGGGTGCGAAGTCGCAACATG-3′

4. Detection—RCA

A mixture corresponding to the following was prepared:
2.66× Thermopol buffer (53.2 mM Tris-HCl, 26.6 mM (NH₄)₂SO₄, 26.6 mM KCl, 5.32 mM MgSO₄, 0.266% Triton® X-100, pH 8.8)
0.28 uM Primer mix 1
284.4 U/m L BST 2.0 WarmStart
14.67 U/m L TIPP
1.06 mM dNTPs
Syto82 dye 3 uM
1.25 uL of reaction mixture from point 3.
Total volume 11.25 uL

Primer mix 1:
Fwd (SEQ ID NO 17):
5′-T*C*GCAACATCCTATATCTGC-3′
Rev (SEQ ID NO 18):
5′-T*G*AGCTTTGACAATACTTGA-3′

where * represents a phosphorothioate bond

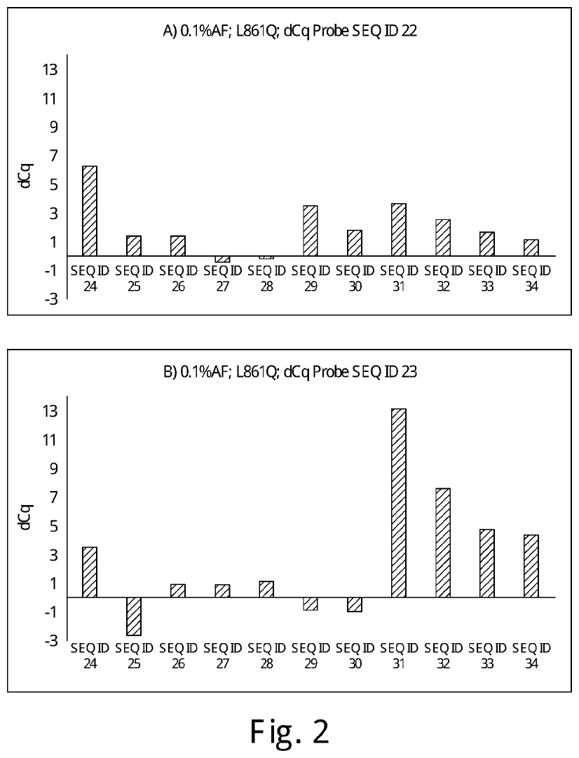

The mixture was then incubated at 50° C. for 90 min using a standard real-time PCR instrument. Fluorescent measurements were taken every 1 minute and a Cq value obtained using the standard instrument manufacturer software. The difference in Cq values (dCq) observed for the wild-type and mutant-containing samples can be seen in FIG. 2.

Summary of the lengths tested can be found in Table 2. For probe SEQ ID 22 (FIG. 2A), the best performing splint sequence is SEQ ID 24. For probe SEQ ID 23 (FIG. 2B), the best performing splint sequence is SEQ ID 31. The closer the position of ligation point to the start of the probe sequence, the larger the value of dCq. There is a larger value of dCq when Probe SEQ ID 23 is used, compared with SEQ ID 22, as it has a longer region of complementarity with the target, 23 base pairs compared with 17 base pairs.

TABLE 2
Complementary between probe and target molecule
and probe and splint and ligation point

| | | | | Length of |
|---|---|---|---|---|
| | Length of | | | complementary |
| | complementary | | Ligation point | region between |
| | region between | | (position from | probe and splint |
| Probe | probe and target | Splint | start of probe | [bp] on probe |
| SEQ ID | molecule [bp] | SEQ ID | sequence) | 3′ end |
| 22 | 17 | 24 | 9 | 6 |
| | | 25 | 16 | 9 |
| | | 26 | 15 | 9 |
| | | 27 | 13 | 10 |
| | | 28 | 13 | 9 |
| | | 29 | 12 | 9 |
| | | 30 | 11 | 9 |
| | | 31 | 10 | 9 |
| | | 32 | 9 | 9 |
| | | 33 | 8 | 9 |
| | | 34 | 7 | 10 |
| 23 | 35 | 24 | 28 | 6 |
| | | 25 | 35 | 9 |
| | | 26 | 34 | 9 |
| | | 27 | 32 | 10 |
| | | 28 | 32 | 9 |
| | | 29 | 31 | 9 |
| | | 30 | 30 | 9 |
| | | 31 | 29 | 9 |
| | | 32 | 28 | 9 |
| | | 33 | 27 | 9 |
| | | 34 | 26 | 10 |

### Example 3: Further Probe/Splint Combinations

1. Polymerase Chain Reaction

A mixture was prepared corresponding to:
1×Q5U buffer
400 nM primer mix 1
20 U/mL Q5U Polymerase
10 U/mL Thermolabile UDG
0.4 ng/uL fragmented human genomic DNA
+/−0.2 aM mutant oligonucleotide
Total volume 50 uL

This mixture was then incubated as follows using a standard thermal cycler:

| 37° | C. | 10 | mins |
|---|---|---|---|
| (98° | C. | 1 | min |
| 98° | C. | 10 | sec |
| 58° | C. | 15 | sec |
| 72° | C. | 15 sec) × 50 | |
| 72° | C. | 5 | mins |

| | | |
|---|---|---|
| 4° | C. | ∞ |

Q5 Buffer

The Q5 buffer is available from commercial supplier NEB.

Primer mix 1:
Fwd (SEQ ID NO 19):
5′-A*C*G*TACTGGTGAAAACACCGC-3′
Rev (SEQ ID NO 20):
5′-/5Phos/GCCTCCTTCTGCATGGTATTCT-3′

where * represents a phosphorothioate bond

Mutant Oligonucleotide (SEO ID NO 21):

5′-ACGTACTGGTGAAAACACCGCAGCATGTCAAGATCACAGATTTT
GGGCTGGCCAAACAGCTGGGTGCGGAAGAGAAAGAATACCATGCAGA
AGGAGGC-3′

Bold and underlined—mutation site

2. Proteinase K Treatment

A mixture was prepared corresponding to:
1×A7
20 U/mL Proteinase K
40 uL of mixture point 1
Total volume 90 uL

This mixture was then incubated at 55° C. for 5 min, 95° C. for 10 min.

1×A7 Composition
Tris Acetate pH=8.0 10 mM
Potassium Acetate 25 mM
Magnesium Acetate 5 mM
Triton-X 0.01%

3. Pyrophosphorolysis (PPL) and Ligation

A mixture was prepared corresponding to:
1×BFF1
10 U/mL Klenow (exo-)
100 U/mL E. coli Ligase
1.2 U/mL apyrase
100 U/mL Lambda exo
0.25 mM PPi
20 nM probe oligonucleotide
30 nM splint oligonucleotide
1.25 uL of mixture from point 2.
Total volume 10 uL

This mixture was then incubated at 45° C. for 15 min.
1×BFF1 composition
Tris Acetate pH=7.0 10 mM
Potassium Acetate 30 mM
Magnesium Acetate 17.125 mM
Triton-X 0.01%

Probe oligonucleotide (SEQ ID NO 22):
/5Phos/A*TGTTCGATGAGCTTTGACAATACTTGATCGATGCAGAT
ATAGGATGTTGCGACGCACCCAGCTGTTTGG
Probe oligonucleotide (SEQ ID NO 23):
/5Phos/A*T*G*TTCGATGAGCTTTGACAATACTTGATCGATGCAG
ATATAGGATGTTGCGACGCACCCAGCTG

where * represents a phosphorothioate bond

Splint oligonucleotide (SEQ ID NO 24):
TTTGGCCAGCCCAAAATCTGTGAT
Splint oligonucleotide (SEQ ID NO 35):
TGTCAAAGCTCATCGAACATGGGTGCGGAAGTCGCT

4. Detection—RCA

A mixture corresponding to the following was prepared:
2.66× Thermopol buffer (53.2 mM Tris-HCl, 26.6 mM (NH₄)₂SO₄, 26.6 mM KCl, 5.32 mM MgSO₄, 0.266% Triton® X-100, pH 8.8)
0.28 uM Primer mix 1
284.4 U/mL BST 2.0 WarmStart
14.67 U/mL TIPP
1.06 mM dNTPs
Syto82 dye 3 uM
1.25 uL of reaction mixture from point 3.
Total volume 11.25 uL

Primer mix 1:
Fwd (SEQ ID NO 17):
5′-T*C*GCAACATCCTATATCTGC-3′
Rev (SEQ ID NO 18):
5′-T*G*AGCTTTGACAATACTTGA-3′

where * represents a phosphorothioate bond

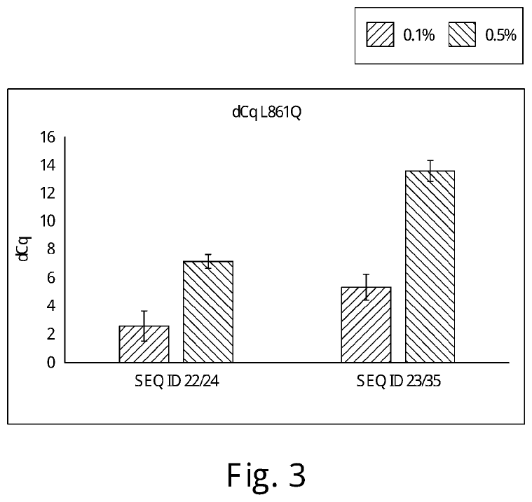

The mixture was then incubated at 50° C. for 90 min using a standard real-time PCR instrument. Fluorescent measurements were taken every 1 minute and a Cq value obtained using the standard instrument manufacturer software. The difference in Cq (dCq) between wildtype and mutant-containing samples can be seen in FIG. 3. Probe SEQ ID 22 and 23 have 17 and 35 base length complementary regions with the target molecule, respectively. The higher the number of complementary bases/longer the length of the complementary region, the higher value of dCq seen for 0.1% and 0.5% AF concentrations of the target molecule.

### Example 4: Further Applications of the Methods of the Invention

Methylation Frequencies of Highly Relevant Methylation Genes (HRMG) in Human Cancers

| Organ | 1 | 2 | 3 | 4 | 5 |
|---|---|---|---|---|---|
| Head and neck SCC | HOXA9 | NID2 | UCHL1 | DCC | KIF1A |
| | (.81, 60%) | (.79, 71%) | (.78, 66%) | (.77, 75%) | (.76, 72%) |
| Esophageal SCC | ZNF582 | NEFH | CDO1 | NMDAR2B | PAX1 |
| | (.95, 86%) | (.93, 86%) | (.91, 84%) | (.91, 78%) | (.89, 100%) |
| Esophageal | SFRP1 | CDO1 | APC | CDH1 | TIMP3 |
| adenocarcinoma | (96%) | (95%) | (92%) | (84%) | (74%) |
| Lung | GHSR | CDO1 | HOXA9 | SHOX2 | TAC1 |
| | (1.0) | (.87, 92%) | (96%) | (94%) | (87%) |
| Stomach | CDO1 | DLEC1 | HOPX | Reprimo | FLNC |
| | (.95, 87%) | (.87, 93%) | (.85, 84%) | (.77, 69%) | (.72, 93%) |
| Large intestine | CDO1 | SFRP1 | GFRA1 | SEPT9 | DCLK1 |
| | (.96, 91%) | (.96, 85%) | (.95, 89%) | (.94, 100%) | (.93, 82%) |
| Biliary tract | SFRP1 | OPCML | CDO1 | ZSCAN18 | DCLK1 |
| | (.95, 84%) | (.93, 89%) | (.91, 85%) | (.77, 65%) | (.75, 58%) |
| Gallbladder | SEPT9 | CDO1 | 14-3-3 sigma | 3-OST-2 | Maspin |
| | (.82, 77%) | (.74, 72%) | (90%) | (72%) | (70%) |
| Pancreas | GHSR | CDO1 | HOPX | NPTX2 | UCHL1 |
| | (1.00) | (.97, 94%) | (.85, 83%) | (100%) | (100%) |
| Breast | GHSR | CDO1 | MAL | 14-3-3 sigma | VGF |
| | (.98, 92%) | (.84, 79%) | (95%) | (91%) | (89%) |
| Uterus (cervical) | NKX6-1 | SOX9 | SOX1 | ZNF516 | LMX1A |
| | (.97, 93%*) | (.96, 92%) | (.95, 88%*) | (.92, 90%) | (.9, 89%*) |
| Uterus | GALR1 | COL14A1 | ZNF177 | ZNF154 | TMEFF2 |
| (endometrial) | (.97, 100%) | (.96, 92%) | (.95, 92%) | (.94, 82%) | (.90, 65%) |
| Bladder | CDO1 | APAF-1 | Twist1 | NID2 | PCDH17 |
| | (.87, 78%) | (100%) | (98%) | (96%) | (92%) |
| Prostate | RASSF1 | MDR1 | APC | GHSR | GSTPI |
| | (.99, 96%) | (.98, 88%) | (.97, 90) | (.97) | (.96, 93%) |

(Area under curve [AUC] of receiver operating characteristic [ROC] curve to differentiate the tumor from the normal counterpart, positive methylation frequency).
Asterisks were assessed by scrape samples.
Area under curve (AUC) could not always endowed in this table due to lack of data.
SCC, squamous cell carcinoma.

Methylation of Lung Cancer Biomarkers

Lung cancer is a leading cause of cancer-associated mortality for a number of reasons, including its late manifestation of symptoms and the low sensitivity of screening techniques such as chest radiography. DNA fragments shed from tumour cells can provide a convenient and minimally invasive access to the molecular portrait of cancer, with these DNA fragments being found in the cell free DNA (cfDNA) isolated from the blood of cancer patients. Cell-free circulating tumour DNA (ctDNA) in the blood plasma presents a surrogate for the entire cancer genome; ctDNA accounts for as low as 0.05% of total cfDNA or less in many cancer patients, especially in the early stages of the disease. These properties make aberrant methylation of ctDNA a promising cancer biomarker, and recent high throughput investigations have demonstrated the correspondence between the changes in methylation profiles of ctDNA and DNA from paired tumour tissue. A list of methylated markers for lung cancer diagnostics and prognosis are shown below in:

| Gene | Marker | Source | Changes in lung cancer |
|---|---|---|---|
| APC | Adenomatous polyposis coli | Plasma | Significantly changes of |
| | | | methylation levels in lung |
| | | | cancer patients compared |
| | | | with healthy subjects |
| CDH13 | Cadherin 13 | * | * |
| DCLK1 | Doublecortin like kinase 1 | * | * |
| DLEC1 | Deleted in lung and esophageal cancer 1 | * | * |
| LINE-1 | LINE-1 retrotransposable element 1 | * | * |
| P16 | Cyclin-dependent kinase inhibitor 2A | * | * |
| (CDKN2A) | | | |
| RARB2 | Retinoic acid receptor beta 2 | * | * |
| SEPT9 | Septin 9 | * | * |
| SHOX2 | Short stature homeobox 2 | * | * |
| BRMS1 | Breast cancer metastasis suppressor 1 | * | Negative impact on survival |
| DCLK1 | Doublecortin like kinase 1 | * | Negative impact on survival |
| LINE1 | LINE-1 retrotransposable element 1 | * | Dynamic changes of methylation |
| | | | levels in response to antitumor |
| | | | therapy |
| APC | Adenomatous polyposis coli | Serum | Significantly changes of |
| | | | methylation levels in lung |
| | | | cancer patients compared |
| | | | with healthy subjects |
| CDH1 | Cadherin 1 | * | * |
| DAPK | Death-associated protein kinase | * | * |
| DCC | DCC netrin 1 receptor | * | * |
| GSTP1 | Glutathione S-transferase pi 1 | * | * |
| MGMT | O-6-methylguanine-DNA | * | * |
| | methyltransferase | | |
| P16 | Cyclin-dependant kinase inhibitor 2A | * | * |
| (CDKN2A) | | | |
| RASSF1A | Ras association domain family 1 | * | * |
| | isoform A | | |
| TMS1 | PYD and CARD domain containing | * | * |
| CHFR | Checkpoint with forkhead and ring | * | Negative impact on survival |
| | finger domains | | with second-line EGFR-TKIs, |
| | | | compared to chemotherapy |
| SFN | Stratifin | * | Positive impact on survival |
| | | | with platinum-based chemotherapy |

Methylation of TERT and MGMT Promoters and Impact on Brain Cancer

The 06-methylguanine-DNA methyltransferase (MGMT) gene encodes an evolutionarily conserved and ubiquitously expressed methyltransferase involved in DNA repair. MGMT removes alkyl adducts from the 06-position of guanine, preventing DNA damage and imparting a protective effect on normal cells. However, the endogenous function of MGMT also protects tumour cells from the otherwise lethal effects of chemotherapy with alkylating agents such as temozolomide (TMZ). Silencing or reduced expression of MGMT through methylation of its respective gene promoter has been observed in 50% of grade IV gliomas, compromising DNA repair and as a result increasing chemosensitivity to agents such as TMZ. Therefore, MGMT promoter methylation status has potential as a biomarker of sensitivity to alkylating chemotherapy, ultimately influencing clinical practice. Its capacity as both a predictive and prognostic biomarker has been studied extensively, however, at present there is no consensus on the optimal method of assessment of MGMT gene promoter methylation.

Telomere maintenance protects the integrity of chromosomal ends, enabling replicative immortality, a hallmark of human cancer. The telomere reverse transcriptase (TERT) oncogene encodes the rate-limiting catalytic subunit of the telomerase holoenzyme, which is responsible for telomere maintenance and is normally only expressed in a subset of stem cells. The TERT gene is reactivated in approximately 90% of cancer cells, allowing indefinite proliferation and immortalisation of these cell types. A variety of genetic and epigenetic mechanisms underlying TERT dysregulation have been identified, with hypermethylation of the TERT promoter region representing a unique characteristic of cancer cells. Interestingly, methylation, and not mutation, in the upstream of transcription start site (UTSS) of the TERT gene was found to be strongly associated with increased TERT expression and poor prognosis in paediatric brain tumours. Given the prevalence of TERT promoter hypermethylation in a wide variety of cancer cell types, this epigenetic modification represents a useful prognostic biomarker.

Methylation of Prostate Cancer Genes Etc. Which Genes are/are not Methylated

Prostate cancer is the most frequently diagnosed non-skin malignancy, and a leading cause of cancer-related death in men in Western industrialised countries. There are many DNA methylation changes observed between benign and cancerous prostate tissues, with changes frequently being early and recurrent, suggesting a potential functional role. Several genes and gene families have been reported to be recurrently hypermethylated in prostate cancer by multiple genome-wide studies. This includes, but is not limited to, GSTP1, MGMT, AR, ER, VHL, RB1, APC, DAPK, CD44, AOX1, APC, CDKN2A, HOXD3, PTGS2, RARB, WT1, ZNF154, C20orf103, EFS, HOXC11, LHX9, RUNX3, TBX15, BARHL2, BDNF, CCDC8, CYP27A1, DLX1, EN2, ESR1, FBLN1, FOXE3, GP5, FRSP, HHEX, HOXA3, HOXD4, HOXD8. IRX1, KIT, LBX1, LHX2, NKX2-1, NKX2-2, NKX2-5, PHOXRA, POU3F3, RHCG, SIX6, TBX3, TMEM106, VAX1, and WNT2.

Methylation of Pancreatic Cancer Markers

Pancreatic ductal adenocarcinoma (PDAC) is one of the most deadly cancer types. This form of cancer is difficult to diagnose as there are currently no early diagnostic tests available, meaning diagnosis usually occurs when the disease is already in an advanced state (>75% of diagnosed cases are stage Ill/IV diseases). This has led to high mortality rates being recorded. Early diagnosis has proven difficult due to the lack of reliable biomarkers able to capture the early development and/or progression of PDAC. Currently, the only FDA approved biomarker for prognostic surveillance of patients with PDAC is carbohydrate antigen 19-9 (CA19-9 or sialylated Lewis antigen). This antigen displays low sensitivity and specificity for the detection of disease. Its use is therefore discouraged for diagnostic purposes unless used in combination with other circulating biomarkers.

Recent studies have shown that cell-free DNA (cfDNA) methylation analysis represents a promising non-invasive approach for the discovery of biomarkers with diagnostic potential. It may be possible for cfDNA methylation to be utilised for the identification of disease-specific signatures in pre-neoplastic lesions or chronic pancreatitis (CP). As CP often precedes PDAC, dynamic DNA methylation patterns for a given set of genes may underlie the progression of the disease. CUX2, a ductal cell marker, has shown increased signal in PDAC; and REG1A, a ductal and acing cell marker, has shown increasing signal in chronic pancreatitis. Biomarkers ADAMTS1 and BNC1 have been seen to have high methylation frequency in primary PDACs and in pre-neoplastic pancreatic intra-epithelial neoplasia (PINs) (25% and 70% for ADAMTS1 and BNC1, respectively). The combined cfDNA methylation of ADAMTS1 and BNC1 may be utilised for the early diagnosis of pancreatic cancers (i.e. stages I and II). A list of potential biomarkers are shown in the below:

Genes Useful to Identify Circulating Exocrine Pancreatic cfDNA

| | | |
|---|---|---|
| CUX2 | Ductal cell marker | Increased signal in PDAC |
| REG1A | Ductal and acing cell marker | Increasing signal in CP |

Frequency of cfDNA methylation biomarkers (%)

| Gene | healthy | CP | PIN | PDAC I | PDAC II | PDAC III/IV |
|---|---|---|---|---|---|---|
| ADAMTS1 | <3 | — | 25 | 25-88 | 78-90 | 55 |
| BNC1 | 3-7 | 5 | 70 | 63-95 | 56-95 | 100 |
| CCND2 | 17 | 82 | — | — | — | 24 |
| CDKN1C | 60 | 90 | — | — | — | 27 |
| MLH1 | 22 | 78 | — | — | — | 27 |
| PGR (prox) | 14 | 76 | — | — | — | 37 |
| SYK | 57 | 89 | — | — | — | 57 |

KRAS Detection

The KRAS gene controls cell proliferation, when it is mutated this negative signalling is disrupted and cells are able to continuously proliferate, often developing into cancer. A single amino acid substitution, and in particular a single nucleotide substitution, is responsible for an activating mutation implicated in various cancers: lung adenocarcinoma, mucinous adenoma, ductal carcinoma of the pancreas and colorectal cancer. KRAS mutations have been used as prognostic biomarkers, for example, in lung cancer.

Driver mutations in KRAS are associated with up to 20% of human cancers and there are targeted therapies in development against this mutation and its associated disease(s), a non-limiting list of some such therapies can be seen in the table below:

| | | | | Brand |
|---|---|---|---|---|
| Name | Target | Manufacturer | Status | Name(s) |
| AMG 510 | KRAS^{G12C} | Amgen | Trials | N/A |
| MRTX-849 | KRAS^{G12C} | Mirati Therapeutics | Trials | N/A |
| ARS-3248 | KRAS^{G12C} | Wellspring Biosciences, | Trials | N/A |
| | | Inc/Janssen Biotech, Inc. | | |

The presence of KRAS mutation has been found to reflect a very poor response to the EGFR inhibitors panitumumab (Vectibix) and cetuximab (Erbitux). Activating mutations in the gene that encodes KRAS occurs in 30%-50% of colorectal cancers and studies show that patients whose tumours express this mutated version of the KRAS gene will not respond to panitumumab and cetuximab. The presence of the wild-type KRAS gene is not a guarantee that a patient will respond to these drugs, however, studies have shown that cetuximab has significant efficacy in metastatic colorectal cancer patients with wild-type KRAS tumours. Lung cancer patients who are positive for KRAS mutation (wild-type EGFR) have a response rate estimated at 5% or less for the EGFR antagonists erlotinib or gefitinib compared with a 60% response rate in patients who do not possess a KRAS mutation.

The early detection of the emergence of KRAS mutations (activating or over-expression), a frequent driver of acquired resistance to cetuximab therapy (anti-EGFR therapy) in colorectal cancers, allows the modification of treatment (for example the early initiation of a mitogen-activated protein kinase kinase [MEK] inhibitor) to delay or reverse resistance and thus it is advantageous that the methods of the current invention allow the quick and cheap detection of the KRAS status of patients.

A non-limiting list of mutations is: G12D, G12A, G12C, G13D, G12V, G12S, G12R, A59T/E/G, Q61H, Q61K, Q61R/L, K117N and A146P/T/V.

A further non-limiting list of mutations is shown in the table below:

| Exon | Mutation name | COSM Number | Mutation sequence |
|---|---|---|---|
| 2 | G12A | COSM522 | c.35G > C |
| 2 | G12C | COSM516 | c.34G > T |
| 2 | G12D | COSM521 | c.35G > A |
| 2 | G12F | COSM512 | c.34_35delinsTT |
| 2 | G12R | COSM518 | c.34G > C |
| 2 | G12S | COSM517 | c.34G > A |
| 2 | G12V | COSM520 | c.35G > T |
| 2 | G12V | COSM515 | c.35_36delinsTC |
| 2 | G13A | COSM533 | c.38G > C |
| 2 | G13C | COSM527 | c.37G > T |
| 2 | G13D | COSM532 | c.38G > A |
| 2 | G13R | COSM529 | c.37G > C |
| 2 | G13S | COSM528 | c.37G > A |
| 3 | Q61E | COSM550 | c.181C > G |
| 3 | Q61H | COSM1146992 | c.183A > T |
| 3 | Q61H | COSM554/COSM1135364 | c.183A > C |
| 3 | Q61K | COSM549/COSM1159597 | c.181C > A |
| 3 | Q61L | COSM553 | c.182A > T |
| 3 | Q61R | COSM552 | c.182A > G |

BRAF Detection

BRAF is a human gene that encodes for a protein called B-Raf which is involved in sending signals inside cells which are involved in directing cell growth. It has been shown to be mutated in some human cancers. B-Raf is a member of the Raf kinase family of growth signal transduction protein kinases and plays a role in regulating the MAP Kinase/ERKs signalling pathway, which affects, amongst other things, cell division.

Certain other inherited BRAF mutations cause birth defects.

More than 30 mutations of the BRAF gene have been identified that are associated with human cancers. In 90% of cases, thymine is substituted with adenine at nucleotide 1799. This leads to valine (V) being substituted for by glutamate (E) at codon 600 (now referred to as V600E) in the activation segment found in human cancers. This mutation has been widely observed in:
Colorectal cancer
Melanoma
Papillary thyroid carcinoma
Non-small cell lung cancer
Ameloblastoma

A non-limiting list of other mutations which have been found are: R4611, 1462S, G463E, G463V, G465A, G465E, G465V, G468A, G468E, N580S, E585K, D593V, F594L, G595R, L596V, T5981, V599D, V599E, V599K, V599R, V600K and A727V.

Drugs that treat cancers driven by BRAF mutations have been developed; vemurafenib and dabrafenib are approved by the FDA for the treatment of late-stage melanoma. The response rate to treatment with vumerafenib was 53% for metastatic melanoma, compared to 7-12% for the former best chemotherapeutic dacarbazine.

ERBB2/HER2 Detection

Human epidermal growth factor receptor 2 (HER2), also known as CD340 (cluster of differentiation 340), proto-oncogene Neu, Erbb2 (rodent) or ERBB2 (human) is a protein encoded by the ERBB2 gene. Amplification or over-expression of this oncogene plays an important role in the progression of aggressive types of breast cancer. Over-expression of the ERBB2 gene is also known to occur in ovarian, stomach, adenocarcinoma of the lung, and aggressive forms of uterine cancer and 30% of salivary duct carcinomas. Structural alterations have also been identified that cause ligand-independent firing of the receptor in the absence of over-expression.

There are numerous targeted therapies approved and in development against this mutation and its associated disease(s), a non-limiting list of some such therapies can be seen in the table below:

| | | | | Brand |
|---|---|---|---|---|
| Name | Target | Manufacturer | Status | Name(s) |
| Trastuzumab | HER2 | Genentech | Approved | Herceptin |
| | overexpression | | | |
| Pertuzumab | Dimerisation of | Genentech | Approved | Perjeta |
| | HER2 and HER3 | | | |
| | receptors | | | |
| Margetuximab | HER2 | Raven | Trials | N/A |
| | overexpression | biotechnologies/MacroGenics | | |
| NeuVax | Vaccine | Galena Biopharma | Trials | N/A |

HER2 testing is routinely performed in breast cancer patients to assess prognosis, monitor response to treatment and to determine suitability for targeted therapy (trastuzumab etc.). As trastuzumab is expensive and associated with serious side effects (cardiotoxicity) it is important that only HER2+ patients are selected to receive it and thus it is advantageous that the methods of the current invention allow the quick and cheap detection of the HER2 status of patients.

In one embodiment, the presence of absence of the ERRB2 Exon 20 insertion mutations is detected using the methods of the current invention.

A further non-limiting list of ERBB2 mutations is shown in the table below:

| Exon | Mutation name | COSM Number | Mutation sequence |
|---|---|---|---|
| 20 | A775_G776insYVMA | COSM12558 | c.2324_2325ins > TTACGTGATGGC |
| | G778_P780dup | COSM12556 | c.2332_2340dup > GGCTCCCCA |
| | G776delinsVC | COSM1651739 | c.2326_2327ins > TGT |
| | P780_Y781insGSP | COSM21607 | c.2339_2340ins > TGGCTCCCC |
| | A775_G776insYVMA/Y772_A775dup | COSM20959 | c.2313_2324dup > ATACGTGATGGC |
| 17 | V659E_AA | COSM3724566 | c.1976_1977delinsTT > AA |
| | V659E_AG | COSM6503262 | ? c.1976_1977delinsTT > AG |

EML4-ALK Detection

EML4-ALK is an abnormal gene fusion of echinoderm microtubule-associated protein-like 4 (EML4) gene and anaplastic lymphoma kinase (ALK) gene. This gene fusion leads to the production of the protein EML4-ALK, which appears to promote and maintain the malignant behaviour of cancer cells. EML4-ALK positive lung cancer is a primary malignant lung tumour whose cells contain this mutation.

There are numerous targeted therapies approved and in development against this mutation and its associated disease(s), a non-limiting list of some such therapies can be seen in the table below:

| Name | Generation | Target | Manufacturer | Status | Brand Name(s) |
|---|---|---|---|---|---|
| Crizotinib | 1 | EML4/ALK | Pfizer | FDA approved 2011 | Xalkori, Crizalk |
| Certinib | 2 | ALK | Novartis | FDA approved 2014 | Zykadia |
| Alectinib | 2 | ALK | Genentec | Japan 2014, FDA | Alecensa |
| | | | | approved 2015 | |
| Brigatinib | 2 | ALK/EGFR | Takeda | FDA approved 2017 | Alunbrig, Briganix |
| Ensartinib | 2 | ALK | XCovery | Trial(s) | N/A |
| Lorlatinib | 3 | ROS1/ALK | Pfizer | Trial(s) | N/A |

EML4-ALK gene fusions are responsible for approximately 5% of non-small cell lung cancers (NSCLC), with about 9,000 new cases in the US per year and about 45,000 worldwide.

There are numerous variants of EML4-ALK with all variants having the essential coiled-coil domain in the EML4 N-terminal portion and in the kinase domain of ALK exon 20 that are needed for transforming activity. Fusion of exon 13 of EML4 with exon 20 of ALK (variant 1: V1), exon 20 of EML4 with exon 20 of ALK (V2), and exon 6 of EML4 with exon 20 of ALK (V3) are some of more common variants. The clinical significance of these different variants has only recently become clearer.

V3 has emerged as a marker suitable for the selection of patients who are likely to have shorter progression-free survival (PFS) after non-tyrosine kinase inhibitor (TKI) treatment such as chemotherapy and radiotherapy. There is further evidence that V3 is associated with shorter PFS of those patients who receive first- and second-generation treatment lines and worse overall survival (OS) compared to V1 and V2 of EML4-ALK.

It has also been found that V3-positive patients develop resistance to first and second treatment lines though the development of resistance mutations and possibly facilitated by incomplete tumour cell suppression due to a higher IC50 of wild-type V3. Detection of the unfavourable V3 could be used to select patients requiring more aggressive surveillance and treatment strategies. It appears that administration of the third generation Lorlatinib to patients with V3 may confer longer PFS over those with V1 and thus it is advantageous that the methods of the current invention allow the quick and cheap detection of which variant a patient may have.

The methods of the current invention further allow the detection of resistance mutations such as, but not limited to: G1202R, G1269A, E1210K, D1203, S1206C, L1196M, F1174C, I1171T, I1171N/S, V1180L, T1151K and C1156Y.

G1202R, for example, is a solvent-front mutation which causes interference with drug binding and confers a high level of resistance to first- and second-generation ALK inhibitors. Thus it is advantageous that the methods of the current invention allow identification of those patients who may possess this mutation and benefit from treatment initiation on a third generation treatment rather than a first or second.

A further non-limiting list of EML4-ALK mutations is shown in the table below:

| | | COSM | Mutation |
|---|---|---|---|
| Exon | Mutation | Number | sequence |
| Rearrangements | EML4-ALK | COSF408 | E13_A20 |
| (always exon 20) | | COSF474 | E6ins33_A20 |
| | | COSF409 | E20_A20 |
| | | COSF411 | E6_A20 |

EGFR Detection

The identification of the epidermal growth factor receptor (EGFR) as an oncogene led to the development of targeted therapies such as gefitinib, erlotinib, afatinib, brigatinib and icotinib for lung cancer, and cetuximab for colon cancer. However, many people develop resistance to these therapies. Two primary sources of resistance are the T790M mutation and the MET oncogene.

EGFR mutations occur in EGFR exons 18-21 and mutations in exons 18, 19 and 21 and indicate suitability for treatment with EGFR-TKIs (tyrosine kinase inhibitors). Mutations in exon 20 (with the exception of a few mutations) show the tumours are EGFR-TKI resistant and not suitable for treatment with EGFR-TKIs.

The two most common EGFR mutations are short in-frame deletions of exon 19 and a point mutation (CTG to CGG) in exon 21 at nucleotide 2573, which results in substitution of leucine by arginine at codon 858 (L858R) Together, these two mutations account for ˜90% of all EGFR mutations in non-small cell lung cancer (NSCLC). Screening for these mutations in patients with NSCLC can be used to predict which patients will respond to TKIs.

Thus it is advantageous that the methods of the current invention allow identification of those patients who may possess these mutations and benefit from treatment initiation on TKIs. The person skilled in the art will appreciate that the methods of the current invention allow identification of a range of EGFR mutations, a non-exhaustive list of such mutations is: G719X, Ex19Del, S768I, Ex201 ns and L861Q.

A further non-limiting list of mutations is shown in the table below:

| | | COSM | Mutation |
|---|---|---|---|
| Exon | Mutation name | Number | sequence |
| Exon 18 | G719A | 6239 | c.2156G > C |
| | G719S | 6252 | c.2155G > A |
| | G719C | 6253 | c.2155G > T |
| Exon 19 | del_6210 | 6210 | 2240_2251del12 |
| | del_6218 | 6218 | 2239_2247del9 |
| | del_6220 | 6220 | 2238_2255del18 |
| | del_6223 | 6223 | 2235_2249del15 |
| | del_6225 | 6225 | 2236_2250del15 |
| | del_6255 | 6255 | 2239_2256del18 |
| | del_12367 | 12367 | 2237_2254del18 |
| | del_12369 | 12369 | 2240_2254del15 |
| | del_12370 | 12370 | 2240_2257del18 |
| | del_12382 | 12382 | 2239_2248TTAAGAGAAG > C |
| | del_12383 | 12383 | 2239_2251 > C |
| | del_12384 | 12384 | 2237_2255 > T |
| | del_12385 | 12385 | 2235_2255 > AAT |
| | del_12386 | 12386 | 2237_2252 > T |
| | del_12387 | 12387 | 2239_2258 > CA |
| | del_12403 | 12403 | 2239_2256 > CAA |
| | del_12416 | 12416 | 2237_2253 > TTGCT |
| | del_12419 | 12419 | 2238_2252 > GCA |
| | del_12422 | 12422 | 2238_2248 > GC |
| | del_12678 | 12678 | 2237_2251del15 |
| | del_12728 | 12728 | 2236_2253del18 |
| | del_13550 | 13550 | 2235_2248 > AATTC |
| | del_13551 | 13551 | 2235_2252 > AAT |
| | del_13552 | 13552 | 2235_2251 > AATTC |
| | del_13556 | 13556 | 2253_2276del24 |
| | del_18427 | 18427 | 2237_2257 > TCT |
| | del_26038 | 26038 | 2233_2247del15 |
| | del_6256 | 6256 | ? c.2254_2277del24 |
| | del_28517 | 28517 | ? c.2235_2246del12 |
| | del_1190791 | 1190791 | ? c.2234_2248del15 |
| | del_26718 | 26718 | ? c.2250_2264del15 |
| | del_133189 | 133189 | ? c.2236_2256del21 |
| | del_24970 | 24970 | ? c.2239_2262del24 |
| | I740_K745dup | 26443 | c.2217_2234dup |
| | K745_E746insVPVAIK | 26444 | c.2219_2236dup |
| Exon 20 | T790M | 6240 | 2369C > T |
| | S768I | 6241 | 2303G > T |
| | p.A767_V769dup | 12376 | 2307_2308ins9GCCAGCGTG |
| | p.H773dup | 12377 | 2319_2320insCAC |
| | p.D770_N771insG | 12378 | 2310_2311insGGT |
| | p.S768_D770dup | 13428 | 2311_2312ins9GCGTGGACA |
| | p.A767_V769dup | 13558 | 2309_2310AC > CCAGCGTGGAT |
| | A763_Y764insFQEA | 26720 | 2284-5_2290dupTCCAGGAAGCCT |
| | C797S_2389 | 6493937 | 2389T > A |
| | C797S_2390 | 5945664 | 2390G > C |
| Exon 21 | L858R | 6224 | c.2573T > G |
| | L858R | 12429 | 2573_2574TG > GT |
| | L861Q | 6213 | c.2582T > A |

ROS1

ROS1 is a receptor tyrosine kinase (encoded by the gene ROS1) with structural similarity to the anaplastic lymphoma kinase (ALK) protein; it is encoded by the c-ros oncogene.

A non-limiting list of ROS1 mutations is shown in the table below:

| | | COSM | Mutation |
|---|---|---|---|
| Exon | Mutation name | Number | sequence |
| Rearrangements | CD74-ROS1 | COSF1202 | C6_R32 |
| | | COSF1200 | C6_R34 |
| | | COSF1478 | C6_R35 |
| | SLC34A2-ROS1 | COSF1196 | S4_R32 |
| | | COSF1198 | S4_R34 |
| | | COSF1259 | S13del2046_R32 |
| | | COSF126 | S13del2046_R34 |
| | SDC4-ROS1 | COSF1265 | S2_R32 |
| | | COSF1278 | S4_R32 |
| | | COSF1671 | S2_R34 |
| | | COSF1280 | S4_R34 |
| | EZR-ROS1 | COSF1267 | E10_R34 |
| | GOPC-ROS1 | — | G4_R35 |
| | | COSF1188 | G4_R36 |
| | | COSF1139 | G8_R35 |
| | LRIG3-ROS1 | COSF1269 | L16_R35 |
| | TPM3-ROS1 | COSF1273 | T8_R35 |

RET Proto-Oncogene

The RET proto-oncogene encodes a receptor tyrosine kinase for members of the glial cell line-derived neurotrophic factor (GDNF) family of extracellular signalling molecules.

A non-limiting list of RET mutations is shown in the table below:

| | | COSM | Mutation |
|---|---|---|---|
| Exon | Mutation name | Number | sequence |
| Rearrangements | KIF5B-RET | COSF1232 | K15-R12 |
| | | COSF1230 | K16-R12 |
| | | COSF1253 | K22-R12 |
| | | COSF1234 | K23-R12 |
| | | COSF1255 | K15-R11 |
| | | (actually 15_11del107) | |
| | | COSF1262 | K24-R11 |
| | | COSF1242 | K24-R8 |
| | | | K24-R7 |
| | CCDC6-RET | COSF1271 | C1-R12 |
| | NCOA4-RET | COSF1341 | N6-R12 |
| | TRIM33-RET | | T14-R12 |

MET Exon 14

MET exon 14 skipping occurs with an approximately 5% frequency in NSCLC and is seen in both squamous and adenocarcinoma histology.

A non-limiting list of MET mutations is shown in the table below:

| | | COSM | Mutation |
|---|---|---|---|
| Exon | Mutation name | Number | sequence |
| Skipping 14 | MET-MET | COSM29312 | M13_M15 |

NTRK Proto-Oncogenes

NTRK gene fusions lead to abnormal proteins called TRK fusion proteins, which may cause cancer cells to grow. NTRK gene fusions may be found in some types of cancer, including cancers of the brain, head and neck, thyroid, soft tissue, lung, and colon. Also called neurotrophic tyrosine receptor kinase gene fusion.

A non-limiting list of NTRK mutations is shown in the table below:

| | | COSM | Mutation |
|---|---|---|---|
| Exon | Mutation name | Number | sequence |
| NTRK1 | CD74-NTRK1 | | C7-N10 |
| Rearrangements | MPRIP-NTRK1 | | M22-N12 |
| | TFG-NTRK1 | COSF1323 | T5-N10 |
| | TPM3-NTRK1 | COSF1329 | T8-N10 |
| NTRK3 | ETV6-NTRK3 | COSF571 | E5-N15 |
| Rearrangements | ETV6-NTRK3 | COSF1534 | E4-N14 |
| | ETV6-NTRK3 | COSF823 | E4-N15 |

Panels

In one embodiment of the invention, there is provided a panel comprising a plurality of probe molecules (A₀) wherein each A₀ is complementary to a target mutation. The mutation may be selected from any mutation previously, or subsequently, described or known. The person skilled in the art will thus appreciate that within the scope of the invention are included panels which may be useful in the detection of one or more mutations to the any of the proto-oncogenes or oncogenes previously, or subsequently, described or known.

In one embodiment, the panel comprises 5-500 individual probe molecules, each complementary to a specific target mutation. In one embodiment, the panel comprises 5-400 individual probe molecules, each complementary to a specific target mutation. In one embodiment, the panel comprises 5-300 individual probe molecules, each complementary to a specific target mutation. In one embodiment, the panel comprises 5-200 individual probe molecules, each complementary to a specific target mutation. In one embodiment, the panel comprises 5-100 individual probe molecules, each complementary to a specific target mutation. In one embodiment, the panel comprises 5-50 individual probe molecules, each complementary to a specific target mutation.

In one embodiment, there may be a plurality of probe molecules specific to the same mutation. In one embodiment, there may be only one probe molecule specific to each mutation of the panel.

In one embodiment, there is provided a panel, wherein the panel comprises a plurality of probe molecules wherein one or more probes are complementary to an EGFR mutation, one or more probes are complementary to a KRAS mutation, one or more probes are complementary to a ERBB2/HER2 mutation, one or more probes are complementary to a EML4-ALK mutation, one or more probes are complementary to a ROS1 mutation, one or more probes are complementary to a RET mutation and one or more probes are complementary to a MET mutation.

In one embodiment, there is provided a panel, wherein the panel comprises a plurality of probe molecules wherein one or more probes may be complementary to an EGFR mutation, one or more probes may be complementary to a KRAS mutation, one or more probes may be complementary to a ERBB2/HER2 mutation, one or more probes may be complementary to a EML4-ALK mutation, one or more probes may be complementary to a ROS1 mutation, one or more probes may be complementary to a RET mutation and one or more probes may be complementary to a MET mutation.

In one embodiment, there is provided a panel of probes selective for one or more EGFR, KRAS, BRAF, ERBB2/HER2, EML4-ALK, ROS1, RET, MET mutations.

In one embodiment, there is provided a panel of probe molecules selective for EGFR mutations.

In one embodiment, there is provided a panel of probe molecules selective for KRAS mutations.

In one embodiment, there is provided a panel of probe molecules selective for BRAF mutations.

In one embodiment, there is provided a panel of probe molecules selective for ERBB2/HER2 mutations.

In one embodiment, there is provided a panel of probe molecules selective for EML4-ALK mutations.

In one embodiment, there is provided a panel of probe molecules selective for ROS1 mutations.

In one embodiment, there is provided a panel of probe molecules selective for RET mutations.

In one embodiment, there is provided a panel of probe molecules selective for NTRK mutations.

In one embodiment, there is provided a panel of probe molecules selective for ROS1 mutations.

In one embodiment, there is provided a panel of probe molecules selective for MET exon 14 mutations.

In one embodiment, there is provided a panel comprising a plurality of probe molecules selective for one or more coding sequences (CDSs).

In one embodiment, there is provided a method of detecting one or more mutations using one or more of the previously described panels.

In one embodiment, there is provided a method of detecting the presence or absence or one or more mutations using one or more of the previously described panels.

In one embodiment, there is provided a kit comprising a panel, which may be as previously or subsequently described, in combination with one or more reagents, which may be as previously or subsequently described.

The person skilled in the art will appreciate that embodiments of kits that disclose A₀, include within their scope embodiments wherein there is a panel comprising a plurality of A₀.

The person skilled in the art will appreciate that the disclosure of the application further encompasses embodiments of panels including capture oligonucleotides B₀. This includes embodiments wherein A₀ and B₀ are regions of the same oligonucleotide Co.

In one embodiment, there is provided a methylation detection panel.

In one embodiment, there is provided a methylation detection kit.

Companion Diagnostics

The methods of the present invention can be used to detect specific genetic markers in a sample which may be used to help guide the selection of appropriate therapy. These markers may be tumour-specific mutations, or may be wild-type genomic sequences, and may be detected using tissue, blood or any other patient sample type. The markers may be epigenetic markers.

Resistance Monitoring

Repeated testing of patient samples during treatment of disease may allow early detection of developed resistance to therapy. As an example of this application is in non-small cell lung carcinoma (NSCLC), in which epidermal growth factor receptor (EGFR) inhibitors (e.g. gefitinib, erlotinib) are commonly used as first line treatments. During treatment the tumour can often develop mutations in the EGFR gene (e.g. T790M, C797S) which confer resistance to the drug. Early detection of these mutations may allow for transfer of the patient onto alternative therapies (e.g. Tagrisso). Epigenetic changes to the DNA of a patient can indicate the development of resistance.

Typically patients being monitored for resistance onset can be too sick for repeated tissue biopsy to be carried out. Repeated tissue biopsy may also be expensive, invasive and carries associated risks. It is preferable to test from blood, but there may be very low copy numbers of the mutations of interest in a reasonable blood drawn sample. Monitoring therefore requires sensitive testing from blood samples using a method of the present invention in which the method is simple and cost effective to carry out such that it can be regularly performed.

Recurrence Monitoring

In this application example, patients who have been declared free of disease following treatment may be monitored over time to detect the recurrence of disease. This needs to be done non-invasively and requires sensitive detection of target sequences from blood samples. By using the method of the present invention, it provides a simple and low-cost method that can be regularly performed. The sequences targeted may be generic mutations known to be common in the disease of interest, or can be custom panels of targets designed for a specific patient based on detection of variants in the tumour tissue prior to remission.

Minimal Residual Disease (MRD) Monitoring

For some cancers there are residual cancer cells that remain in a patient after treatment, it is a major cause of relapse in cancer and leukaemia. MRD monitoring and testing has several important roles: determining whether treatment has eradicated the cancer or whether traces remain, comparing the efficacy of different treatments, monitoring patient remission status as well as detecting recurrence of leukaemia, and choosing the treatment that will best meet those needs.

Screening

Population screening for early detection of disease has been a long-held goal, particularly in cancer diagnostics. The challenge is two-fold: the identification of panels of markers which allow confident detection of disease without too many false negatives, and the development of a method with sufficient sensitivity and low enough cost. The methods of the present invention could be used to address larger panels of mutations than PCR-based tests but with a much simpler workflow and lower cost than sequencing-based diagnostics.

Organ Transplant Rejection

When a transplanted organ is rejected by the recipient, the DNA from this organ is shed into the recipient's bloodstream. Early detection of this DNA would allow early detection of rejection. This could be achieved using custom panels of donor-specific markers, or by using panels of variants known to be common in the population, some of which will be present in the donor and some in the recipient. Routine monitoring of organ recipients over time could be enabled by the low cost and simple workflow of the present invention disclosed herein.

Non-Invasive Prenatal Testing (NIPT)

It has long been known that foetal DNA is present in maternal blood, and the NIPT market is now quite saturated with companies using sequencing the identify mutations and count copy numbers of specific chromosomes to enable detection of foetal abnormalities. The methods of the present invention as disclosed herein have the ability to detect mutations at very low allele fractions, potentially allowing earlier detection of foetal DNA. Identification of common mutations in a given population would allow assays to be developed that target mutations that may be present in either the maternal or foetal DNA or to allow detection of abnormalities at an earlier stage of pregnancy.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments, it is not limited to the disclosed embodiments and that alternative embodiments could be constructed without departing from the scope of the invention as defined in the appended claims.

The person skilled in the art will understand that references to ‘partially digested strand A₁’ may refer to the single-stranded oligonucleotide formed by progressive digestion of A₀ when hybridised to a target analyte sequence, in the 3′-5′ direction until the strands dissociate due to lack of complementarity.

The person skilled in the art will understand that references to ‘partially double-stranded’ nucleic acids may refer to nucleic acids wherein one or more portions are double-stranded and one or more portions are single-stranded.

The person skilled in the art will understand that references to ‘substantially double-stranded’ nucleic acids may refer to nucleic acids wherein one or more portions are double-stranded and one or more smaller portions are single-stranded.

1. A panel comprising a plurality of molecular systems for detecting multiple target polynucleotide sequences in a given nucleic acid analyte, each of the plurality of molecular systems comprising a probe molecule (A₀) and a hybridised splint molecule (C), wherein:
a. each A₀ has a varying 3′-end which is complementary to one of the target polynucleotide sequences, a loop region and a 5′-phosphate; and
b. C is hybridised to the 5′-end of A₀ and provides a single stranded 3′-overhang having a varying sequence,
wherein the single stranded 3′-overhang can hybridise to a region located within the loop region 1 to 50 bases in the 5′ direction from the 3′-end of A₀.

2. The panel of claim 1 wherein the 5′ end of A₀ is resistant to exonucleolysis.

3. The panel of claim 1 or 2 wherein A₀ and C are hybridised at the 5′-end of A₀ across a region comprising a minimum of 5 complementary nucleotides.

4. The panel of claim 1, 2 or 3 wherein the single stranded 3′-overhang of C is complementary to a region located 1-50 bases in the 5′ direction from the 3′-end of A₀ across a region comprising a minimum of 5 complementary nucleotides.

5. The panel of claim 3 or 4 wherein the complementary regions are a minimum of 7 nucleotides in length.

6. The panel of any one preceding claim wherein the 3′ end of A₀ is complementary to a region of a gene or chromosome within the DNA or RNA of a cancerous tumour cell.

7. The panel of claim 6 wherein the 3′ end of A₀ is complementary to a region of a gene encoding a mutation found in non-small cell lung cancer (NSCLC).

8. A method for detecting target polynucleotide sequences in a given nucleic acid analyte, the method comprising taking a panel of molecular systems as claimed in any one of claims 1 to 7 and:
i) introducing a sample to a reaction mixture comprising the panel of molecular systems;
ii) treating A₀ with an enzyme for pyrophosphorolysis thereby removing complementary nucleotides from the 3′-ends of A₀ that are fully hybridised to the targets, to form shortened probes A₁;
iii) using C to displace the 3′-ends of A₁ from the target;
iv) ligating the ends of A₁ to form circles using the pre-hybridised C, to form circular A₂; and
v) detecting the presence of A₂.

9. The method of claim 8, wherein the target polynucleotide comprises a site of genetic mutation and this mutation is present at a low level in the sample compared to the wild-type sequences.

10. The method of claim 8 or 9, wherein A₂ is between 20 and 200 nucleotides in length.

11. The method of claim 10, wherein A₂ is between 40 and 100 nucleotides in length.

12. The method of any one of claims 9 to 11, wherein after (iv) an exonuclease is used to digest any non-circularised nucleic acid material.

13. The method of any one of claims 9 to 12, wherein A₀ has a 5′ end which is resistant to exonucleolysis and wherein a 5′-3′ exonuclease is used to digest any nucleic acid molecules which are not rendered resistant to this exonucleolysis.

14. The method of claim 13, wherein the exonuclease used has activity at least partly dependent on the presence of a 5′ phosphate group and in that the digestion is carried out in the presence of a kinase and a phosphate donor.

15. The method as claimed in any one of claims 9 to 14, wherein step (ii) is carried out in the presence of a phosphatase.

16. The method as claimed in any one of claims 9 to 15, wherein the pyrophosphorolysis reaction is stopped after step (ii) through addition of a pyrophosphatase.

17. The method as claimed in any one of claims 9 to 16, wherein following step (iv) the detection of A₂ is via nucleic acid amplification.

18. The method as claimed in any one of claims 9 to 17, wherein step (v) comprises using one or more oligonucleotide binding dyes or molecular probes.

19. The method as claimed in any one of claims 9 to 18, wherein multiple molecular systems are employed, each comprising A₀ selective for a different target sequence and each A₀ includes an identification region.

20. The method as claimed in claim 19 the identification regions(s) are characterised using molecular probes or through sequencing.

21. The method as claimed in claim 19 wherein the identification region(s) are used as priming sites for nucleic acid amplification, enabling detection and identification of A₂.

22. A method as claimed in claim 20 wherein (v) further comprises the steps of:
viii. labelling the amplification products from A₂ using one or more oligonucleotide fluorescent binding dyes or molecular probes;
ix. measuring the fluorescent signal;
x. exposing the amplification products from A₂ to a set of denaturing conditions; and
xi. identifying the polynucleotide target sequence in the analyte by monitoring changes in the fluorescent signal during exposure to the denaturing conditions.

23. A method as claimed in claims 9 to 22 wherein the different probes A₀ comprise a common priming site for amplification, allowing a single or single set of amplification primers to be used.

24. A kit comprising the molecular systems of any one of the claims 1 to 7 and:
a ligase;
a pyrophosphorylising enzyme;
a source of ions suitable for driving the pyrophosphorolysis reaction; and
suitable buffers.

25. A kit as claimed in claim 24 further comprising dNTPs and a polymerase.

26. A kit as claimed in claim 25, further comprising primers for amplification of region(s) including the target nucleic acid sequence(s).

27. A kit as claimed in claim 26, further comprising a reverse transcriptase.

28. A kit as claimed in claims 25 to 27 further comprising dUTP and UDG.


SEQUENCE LISTING (38 sequences)

SEQ ID NO: 1 - DNA, 77 bp, Artificial Sequence
  Probe
  * (1)..(4): Phosphorothioate bonds between bases 1&2, 2&3, 3&4
  5Phos (1)..(1): 5′ end phosphorylation
atgttcgatgagctttgacaatacttgacatgcgcagatataggatgttgccgaacgcaccggaggccagcactttg

SEQ ID NO: 2 - DNA, 36 bp, Artificial Sequence
  Splint oligonucleotide
tgtcaaagctcatcgaacatccggtgcgttcggcat

SEQ ID NO: 3 - DNA, 77 bp, Artificial Sequence
  Probe
  * (1)..(4): Phosphorothioate bonds between bases 1&2, 2&3, 3&4
  5Phos (1)..(1): 5′ end phosphorylation
atgttcgatgagctttgacaatacttgacatgcgcagatataggatgttgcggaacgcaccggaggccagcactttg

SEQ ID NO: 4 - DNA, 36 bp, Artificial Sequence; identical to SEQ ID NO: 2
  Splint oligonucleotide

SEQ ID NO: 5 - DNA, 77 bp, Artificial Sequence
  Probe
  * (1)..(4): Phosphorothioate bonds between bases 1&2, 2&3, 3&4
  5Phos (1)..(1): 5′ end phosphorylation
atgttcgatgagctttgacaatacttgacatgcgcagatataggatgttgcgaaacgcaccggaggccagcactttg

SEQ ID NO: 6 - DNA, 36 bp, Artificial Sequence
  Splint oligonucleotide
tgtcaaagctcatcgaacatccggtgcgttcggcaa

SEQ ID NO: 7 - DNA, 77 bp, Artificial Sequence
  Probe
  * (1)..(4): Phosphorothioate bonds between bases 1&2, 2&3, 3&4
  5Phos (1)..(1): 5′ end phosphorylation
atgttcgatgagctttgacaatacttgacatgcgcagatataggatgttgcgatacgcaccggaggccagcactttg

SEQ ID NO: 8 - DNA, 36 bp, Artificial Sequence
  Splint oligonucleotide
tgtcaaagctcatcgaacatccggtgcgtatcgcat

SEQ ID NO: 9 - DNA, 77 bp, Artificial Sequence
  Probe
  * (1)..(4): Phosphorothioate bonds between bases 1&2, 2&3, 3&4
  5Phos (1)..(1): 5′ end phosphorylation
atgttcgatgagctttgacaatacttgacatgcgcagatataggatgttgcgattcgcaccggaggccagcactttg

SEQ ID NO: 10 - DNA, 36 bp, Artificial Sequence
  Splint oligonucleotide
tgtcaaagctcatcgaacatccggtgcgaatcgcat

SEQ ID NO: 11 - DNA, 77 bp, Artificial Sequence
  Probe
  * (1)..(4): Phosphorothioate bonds between bases 1&2, 2&3, 3&4
  5Phos (1)..(1): 5′ end phosphorylation
atgttcgatgagctttgacaatacttgacatgcgcagatataggatgttgcgattggcaccggaggccagcactttg

SEQ ID NO: 12 - DNA, 36 bp, Artificial Sequence
  Splint oligonucleotide
tgtcaaagctcatcgaacatccggtgccaatcgcat

SEQ ID NO: 13 - DNA, 77 bp, Artificial Sequence
  Probe
  * (1)..(4): Phosphorothioate bonds between bases 1&2, 2&3, 3&4
  5Phos (1)..(1): 5′ end phosphorylation
atgttcgatgagctttgacaatacttgacatgcgcagatataggatgttgcgattgccaccggaggccagcactttg

SEQ ID NO: 14 - DNA, 36 bp, Artificial Sequence
  Splint oligonucleotide
tgtcaaagctcatcgaacatccggtggcaatcgcat

SEQ ID NO: 15 - DNA, 77 bp, Artificial Sequence
  Probe
  * (1)..(4): Phosphorothioate bonds between bases 1&2, 2&3, 3&4
  5Phos (1)..(1): 5′ end phosphorylation
atgttcgatgagctttgacaatacttgacatgcgcagatataggatgttgcgattgcgaccggaggccagcactttg

SEQ ID NO: 16 - DNA, 36 bp, Artificial Sequence
  Splint oligonucleotide
tgtcaaagctcatcgaacatccggtcgcaatcgcat

SEQ ID NO: 17 - DNA, 20 bp, Artificial Sequence
  Primer mix 1 Fwd
  * (1)..(3): Phosphorothioate bonds between bases 1&2, 2&3
tcgcaacatcctatatctgc

SEQ ID NO: 18 - DNA, 20 bp, Artificial Sequence
  Primer mix 1 Rev
  * (1)..(3): Phosphorothioate bonds between bases 1&2, 2&3
tgagctttgacaatacttga

SEQ ID NO: 19 - DNA, 21 bp, Artificial Sequence
  Primer mix 1 Fwd
  * (1)..(4): Phosphorothioate bonds between bases 1&2, 2&3, 3&4
acgtactggtgaaaacaccgc

SEQ ID NO: 20 - DNA, 22 bp, Artificial Sequence
  Primer mix 1 Rev
  5Phos (1)..(1): 5′ end phosphorylation
gcctccttctgcatggtattct

SEQ ID NO: 21 - DNA, 98 bp, Artificial Sequence
  Mutant oligonucleotide
acgtactggtgaaaacaccgcagcatgtcaagatcacagattttgggctggccaaacagctgggtgcggaagagaaagaataccatgcagaaggaggc

SEQ ID NO: 22 - DNA, 70 bp, Artificial Sequence
  Probe oligonucleotide
  * (1)..(2): Phosphorothioate bond between bases 1&2
  5Phos (1)..(1): 5′ end phosphorylation
atgttcgatgagctttgacaatacttgatcgatgcagatataggatgttgcgacgcacccagctgtttgg

SEQ ID NO: 23 - DNA, 89 bp, Artificial Sequence
  Probe oligonucleotide
  * (1)..(4): Phosphorothioate bonds between bases 1&2, 2&3, 3&4
  5Phos (1)..(1): 5′ end phosphorylation
atgttcgatgagctttgacaatacttgatcgatgcagatataggatgttgcgacgcacccagctgtttggccagcccaaaatctgtgat

SEQ ID NO: 24 - DNA, 36 bp, Artificial Sequence
  Splint oligonucleotide
tgtcaaagctcatcgaacattgggtgtcgcaacatg

SEQ ID NO: 25 - DNA, 43 bp, Artificial Sequence
  Splint oligonucleotide
tgtcaaagctcatcgaacatgtcgcaacagaagtcgcaacatg

SEQ ID NO: 26 - DNA, 43 bp, Artificial Sequence
  Splint oligonucleotide
tgtcaaagctcatcgaacatcgtcgcaacgaagtcgcaacatg

SEQ ID NO: 27 - DNA, 43 bp, Artificial Sequence
  Splint oligonucleotide
tgtcaaagctcatcgaacatgcgtcgcaagaagtcgcaacatg

SEQ ID NO: 28 - DNA, 43 bp, Artificial Sequence
  Splint oligonucleotide
tgtcaaagctcatcgaacattgcgtcgcagaagtcgcaacatg

SEQ ID NO: 29 - DNA, 43 bp, Artificial Sequence
  Splint oligonucleotide
tgtcaaagctcatcgaacatgtgcgtcgcgaagtcgcaacatg

SEQ ID NO: 30 - DNA, 43 bp, Artificial Sequence
  Splint oligonucleotide
tgtcaaagctcatcgaacatggtgcgtcggaagtcgcaacatg

SEQ ID NO: 31 - DNA, 43 bp, Artificial Sequence
  Splint oligonucleotide
tgtcaaagctcatcgaacatgggtgcgtcgaagtcgcaacatg

SEQ ID NO: 32 - DNA, 43 bp, Artificial Sequence
  Splint oligonucleotide
tgtcaaagctcatcgaacattgggtgcgtgaagtcgcaacatg

SEQ ID NO: 33 - DNA, 43 bp, Artificial Sequence
  Splint oligonucleotide
tgtcaaagctcatcgaacatctgggtgcggaagtcgcaacatg

SEQ ID NO: 34 - DNA, 43 bp, Artificial Sequence
  Splint oligonucleotide
tgtcaaagctcatcgaacatgctgggtgcgaagtcgcaacatg

SEQ ID NO: 35 - DNA, 36 bp, Artificial Sequence
  Splint oligonucleotide
tgtcaaagctcatcgaacatgggtgcggaagtcgct

SEQ ID NO: 36 - DNA, 25 bp, Artificial Sequence
  Primer mix 1 Fwd
  * (1)..(4): Phosphorothioate bonds between bases 1&2, 2&3, 3&4
cccaaccaagctctcttgaggatct

SEQ ID NO: 37 - DNA, 24 bp, Artificial Sequence
  Primer mix 1 Rev
  5Phos (1)..(1): 5′ end phosphorylation
gggaccttaccttatacaccgtgc

SEQ ID NO: 38 - DNA, 100 bp, Artificial Sequence
  Mutant oligonucleotide
cccaaccaagctctcttgaggatcttgaaggaaactgaattcaaaaagatcaaagtgctggcctccggtgcgttcggcacggtgtataaggtaaggtccc